(19) United States
(12) Patent Application Publication
(10) Pub. No.: US 2008/0293704 A1
(43) Pub. Date: Nov. 27, 2008

(54) FACTOR Xa INHIBITORS

(75) Inventors: Zhaozhong J. Jia, San Mateo, CA (US); Yonghong Song, Foster City, CA (US); Robert M. Scarborough, Half Moon Bay, CA (US); Carroll Scarborough, Half Moon Bay, CA (US); Anjali Pandey, Fremont, CA (US)

Correspondence Address: FOLEY & LARDNER LLP, 975 PAGE MILL ROAD, PALO ALTO, CA, 94304, US

(73) Assignee: Millennium Pharmaceuticals, Inc.

(21) Appl. No.: 11/969,758

(22) Filed: Jan. 4, 2008

Related U.S. Application Data
(60) Provisional application No. 60/883,734, filed on Jan. 5, 2007.

(51) Int. Cl. A61K 31/4439; C07D 409/14; A61K 31/4545; A61K 31/541; A61P 7/00; A01N 1/00; A61K 31/496; C07D 279/12

(52) U.S. Cl. 514/227.8; 546/268.4; 546/194; 544/364; 544/58.6; 544/58.2; 514/340; 514/318; 514/253.09; 435/2

(57) ABSTRACT

The present invention is directed to compounds represented by Formula (I) or a pharmaceutically acceptable salt, ester, or prodrug thereof which are inhibitors of Factor Xa. The present invention is also directed to intermediates used in making such compounds, pharmaceutical compositions containing such compounds, methods to prevent or treat certain conditions characterized by undesired thrombosis and methods of inhibiting the coagulation of a blood sample.

### CROSS-REFERENCE TO RELATED APPLICATION

This application claims the benefit under 35 U.S.C. §119(e) to U.S. provisional application Ser. No. 60/883,734, filed on Jan. 5, 2007, which is incorporated herein by reference in its entirety.

### BACKGROUND OF THE INVENTION

1. Field of the Invention

This invention is directed to substituted triazole compounds which act as inhibitors of Factor Xa. This invention is also directed to pharmaceutical compositions containing the substituted triazole compounds and methods of using the compounds or compositions to treat a condition characterized by undesired thrombosis. The invention is also directed to methods of making the compounds described herein.

2. State of the Art

Hemostasis, the control of bleeding, occurs by surgical means, or by the physiological properties of vasoconstriction and coagulation. This invention is particularly concerned with blood coagulation and ways in which it assists in maintaining the integrity of mammalian circulation after injury, inflammation, disease, congenital defect, dysfunction or other disruption. Although platelets and blood coagulation are both involved in restoring hemostasis and in thrombotic diseases, certain components of the coagulation cascade are primarily responsible for the amplification and acceleration of the processes involved in platelet aggregation and fibrin deposition which are major events in thrombosis and hemostasis.

Clot formation involves the conversion of fibrinogen to fibrin which polymerizes into a network to restore hemostasis after injury. A similar process results in occluded blood vessels in thrombotic diseases. The conversion of fibrinogen to fibrin is catalyzed by thrombin, the end product of a series of reactions in the blood coagulation cascade. Thrombin is also a key player in activating platelets, thereby contributing to thrombosis under conditions of both arterial and venous blood flow. For these reasons, it has been postulated that efficient regulation of thrombin can lead to efficient regulation of thrombosis. Several classes of currently used anticoagulants directly or indirectly affect thrombin (e.g. unfractionated heparins, low-molecular weight heparins, heparin-like compounds, pentasaccharide and warfarin). Direct or indirect inhibition of thrombin activity has also been the focus of a variety of anticoagulants in clinical development (reviewed by Eriksson and Quinlan, Drugs 11: 1411-1429, 2006).

Prothrombin, the precursor for thrombin, is converted to the active enzyme by factor Xa. Localized activation of tissue factor/factor VIIa mediated factor Xa generation is amplified by the factor IXa/factor VIIIa complex and leads to prothrombinase assembly on activated platelets. Factor Xa, as a part of the prothrombinase complex, is the sole enzyme responsible for sustained thrombin formation in the vasculature. Factor Xa is a serine protease, the activated form of its precursor Factor X, and a member of the calcium ion binding, gamma carboxyglutamic acid (GLA)-containing, vitamin K dependent, blood coagulation factors. Unlike thrombin, which acts on a variety of protein substrates including fibrinogen and the PAR receptors (Protease activated receptors, Coughlin, J Thrombosis Haemostasis 3: 1800-1814, 2005), factor Xa appears to have a single physiologic substrate, namely prothrombin. Since one molecule of factor Xa may be able to generate greater than 1000 molecules of thrombin (Mann, et al., J. Thrombosis. Haemostasis 1: 1504-1514, 2003), direct inhibition of factor Xa as a way of indirectly inhibiting the formation of thrombin is considered an efficient anticoagulant strategy. This assertion is based on the key role of prothrombinase in thrombin synthesis and on the fact that inhibition of prothrombinase will have a pronounced effect on the overall platelet aggregation and clotting pathways. Activated proteases such as factor VIIa, factor IXa or factor Xa have poor proteolytic activity on their own. However, their assembly into cofactor-dependent, membrane-bound complexes significantly enhances their catalytic efficiencies. This effect is most dramatic for factor Xa, where the efficiency is increased by a factor of 10⁵ (Mann, et al., Blood 76(1):1-16, 1990). Due to the higher concentration of the zymogens present in blood (1.4 micromolar prothrombin versus 150 nanomolar factor X) and the kinetics of activation, a smaller amount of factor Xa than thrombin needs to be inhibited to achieve an anticoagulant effect. Indirect proof of the hypothesis of superiority of factor Xa as a therapeutic target compared to thrombin can also be found in clinical trials for the prevention of deep vein thrombosis. Fondaparinux, an antithrombin III dependent factor Xa inhibitor, was proven to be superior to enoxaparin (a low molecular weight heparin that inhibits both thrombin and factor Xa) in four trials of orthopedic surgery (Turpie, et al., Archives Internal Medicine 162(16): 1833-1840, 2002). Therefore, it has been suggested that compounds which selectively inhibit factor Xa may be useful as in vitro diagnostic agents, or for therapeutic administration in certain thrombotic disorders, see e.g., WO 94/13693.

Several Factor Xa inhibitors have been reported as polypeptides derived from hematophagous organisms, as well as compounds which are not large polypeptide-type inhibitors. Additional Factor Xa inhibitors include small molecule organic compounds, such as nitrogen containing heterocyclic compounds which have amidino substituent groups, wherein two functional groups of the compounds can bind to Factor Xa at two of its active sites. For example, WO 98/28269 describes pyrazole compounds having a terminal C(═NH)—NH₂ group; WO 97/21437 describes benzimidazole compounds substituted by a basic radical which are connected to a naphthyl group via a straight or branched chain alkylene, —C(═O) or —S(═O)₂ bridging group; WO 99/10316 describes compounds having a 4-phenyl-N-alkylamidino-piperidine and 4-phenoxy-N-alkylamidino-piperidine group connected to a 3-amidinophenyl group via a carboxamidealkyleneamino bridge; and EP 798295 describes compounds having a 4-phenoxy-N-alkylamidino-piperidine group connected to an amidinonaphthyl group via a substituted or unsubstituted sulfonamide or carboxamide bridging group. Additional reported Factor Xa inhibitors include those having a structure comprising a phenyl-amidino, phenyl and halo-phenyl connected via amide linkages (U.S. Pat. No. 6,844,367 B1). Other Factor Xa inhibitors by the same group have replaced the halo-phenyl with a halo-pyridyl (see U.S. Pat. Nos. 6,376,515 B2 and 6,835,739 B2).

There exists a need for effective therapeutic agents for the regulation of hemostasis, and for the prevention and treatment of thrombus formation and other pathological processes in the vasculature induced by thrombin such as restenosis and inflammation. In particular, there continues to be a need for compounds which selectively inhibit factor Xa or its precursors. Compounds that have different combinations of bridging groups and functional groups than compounds previously discovered are needed, particularly compounds which selectively or preferentially bind to Factor Xa. Compounds with a higher degree of binding to Factor Xa than to thrombin are desired, especially those compounds having good bioavailability and/or solubility.

### BRIEF SUMMARY OF THE INVENTION

The present invention provides in one embodiment, a compound having Formula (I) or a pharmaceutically acceptable salt, ester, or prodrug thereof:

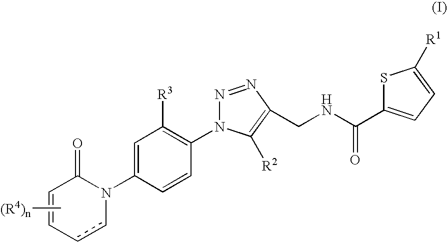

wherein
R¹ is halogen;
R² is hydrogen or halogen;
R³ is selected from the group consisting of —NO₂, —NR^{5a}R^{5b}, -L-NR^{5a}R^{5b}, —NHC(O)NR^{5a}R^{5b}, —NHC(O)R^{5c}, —NHC(O)Y, C₁₋₆ alkyl, —CO₂H, —C(O)NR^{5a}R^{5b}, —C(O)NR^{5a}Y, —C(O)NH-L-Y, —OH, C₁₋₆ alkoxy, —O-L-NR^{5a}R^{5b}, —O-L-O—C(O)NR^{5a}R^{5b}, —Y, —O—Y, —O-L-Y, —O-L-Y-L-Y, and —S(O)ₚR^{5c}, wherein said C₁₋₆ alkyl and C₁₋₆ alkoxy are optionally substituted with one to three substituents selected from R⁶;
R⁴ is independently selected from the group consisting of halogen, —OH, —O-L-Y, —O-L-NR^{5a}R^{5b}, and C₁₋₆ alkoxy optionally substituted with one to three substituents selected from R⁶;
L is C₁-C₄ alkylene;
Y is aryl, heteroaryl, or heterocyclic ring, wherein said aryl and heteroaryl are optionally substituted with one to three R⁶ and said heterocyclic ring is optionally substituted with oxo and optionally with one to three R⁶ or R⁸;
R^{5a} and R^{5b} are independently hydrogen or C₁₋₈ alkyl optionally substituted with one to three R⁶, or R^{5a} and R^{5b} together with the nitrogen atom to which they are both attached to form a 5 to 7 membered heterocyclic ring optionally having one additional ring heteroatom selected from N, NR⁶, O, and S(O)ₚ and where said ring is optionally substituted with one to three substituents selected from R⁶;
R^{5c} is C₁₋₈ alkyl optionally substituted with one to three R⁶;
R⁶ is independently selected from the group consisting of halogen, —OH, —R⁷, —OR⁷, oxo, —SR⁷, —S(O)R⁷, —S(O)₂R⁷, —SO₂NH₂, —C(O)NH₂, —C(O)R⁷, —C(NH)R⁷, —NHC(O)R⁷, —NHC(NH)R⁷, —NHC(O)NH₂, —CO₂H, —NH₂, —NHR⁷, —N(R⁷)₂;
R⁷ is independently C₁₋₆ alkyl;
R⁸ is -L-heteroaryl optionally substituted with one to three substituents selected from R⁶;
   n is 0, 1, or 2;
   p is 0, 1, or 2; and
dashed lines [symbol] are independently single or double bonds;
provided that R³ is not

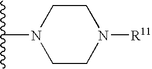

where R¹¹ is hydrogen or alkyl.

The present invention further provides chemical intermediates, pharmaceutical compositions and methods for preventing or treating a condition in a mammal characterized by undesired thrombosis comprising the step of administering to said mammal a therapeutically effective amount of a compound of the present invention. Such conditions include but are not limited to acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, and the like.

The present invention further provides methods for inhibiting the coagulation of a blood sample comprising contacting said sample with a compound of the present invention.

These and other embodiments of the present invention are further described in the text that follows.

### DETAILED DESCRIPTION OF THE INVENTION

### Abbreviations and Definitions

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group is one having one or more, preferably 1 to 3, double bonds. Similarly, the term “alkynyl” refers to an unsaturated alkyl group having one or more, preferably 1 to 3, triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “cycloalkyl” refers to hydrocarbon rings having the indicated number of ring atoms (e.g., C₃₋₆ cycloalkyl) and being fully saturated between ring vertices. The term “cycloalkenyl” refers to a cycloalkyl group that has at least one point of alkenyl unsaturation between the ring vertices. The term “cycloalkynyl” refers to a cycloalkyl group that has at least one point of alkynyl unsaturation between the ring vertices. When “cycloalkyl” is used in combination with “alkyl”, as in C₃₋₅ cycloalkyl-alkyl, the cycloalkyl portion is meant to have the stated number of carbon atoms (e.g., from three to five carbon atoms), while the alkyl portion is an alkylene moiety having from one to three carbon atoms (e.g., —CH₂—, —CH₂CH₂— or —CH₂CH₂CH₂—).

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having four or fewer carbon atoms.

The terms “alkoxy,” “alkylamino,” and “alkylthio” (or “thioalkoxy”) are used in their conventional sense, and refer to those alkyl groups attached to the remainder of the molecule via an oxygen atom (—O-alkyl), an amino group, or a sulfur atom (—S-alkyl), respectively. Additionally, for dialkylamino groups (typically provided as —NR^{a}R^{b} or a variant thereof, where R^{a} and R^{b} are independently alkyl or substituted alkyl), the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl up to the maximum number of halogens permitted. For example, the term “C₁₋₄ haloalkyl” is mean to include trifluoromethyl, 2,2,2-trifluoroethyl, 4-chlorobutyl, 3-bromopropyl, and the like.

The term “hydroxy” or “hydroxyl” refers to the group —OH.

The term “aryl” means, unless otherwise stated, a polyunsaturated, aromatic, hydrocarbon group containing from 6 to 14 carbon atoms, which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom or through a carbon atom and can contain 5 to 10 carbon atoms. In embodiments where multiple rings are fused or linked covalently, condensed (e.g., naphthyl or anthryl), not all rings need be aromatic (e.g., 2-benzoxazolinone, 2H-1,4-benzoxazin-3(4H)-one-7-yl, and the like) provided that the point of attachment is at an aromatic ring. Non-limiting examples of aryl groups include phenyl, naphthyl and biphenyl, while non-limiting examples of heteroaryl groups include 1-pyrrolyl, 2-pyrrolyl, 3-pyrrolyl, 1-pyrazolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, benzopyrazolyl, 5-indolyl, 1-isoquinolyl, 5-isoquinolyl, 2-quinoxalinyl, 5-quinoxalinyl, 3-quinolyl, and 6-quinolyl. If not specifically stated, substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl or heteroaryl group is attached to an alkyl group (e.g., benzyl, phenethyl, pyridylmethyl and the like).

The term “heterocycle” or “heterocyclyl” or “heterocyclic” refers to a saturated or unsaturated non-aromatic cyclic group containing at least one sulfur, nitrogen or oxygen heteroatom. Each heterocycle can be attached at any available ring carbon or heteroatom. Each heterocycle may have one or more rings. When multiple rings are present, they can be fused together or linked covalently, and one or more the rings can be cycloalkyl, aryl or heteroaryl provided that the point of attachment is through the heterocyclic ring. Each heterocycle must contain at least one heteroatom (typically 1 to 5 heteroatoms) selected from nitrogen, oxygen or sulfur. Preferably, these groups contain 1-10 carbon atoms, 0-5 nitrogen atoms, 0-2 sulfur atoms and 0-2 oxygen atoms, wherein the sulfur atoms are optionally oxidized and the nitrogen atoms are optionally quaternized. More preferably, these groups contain 0-3 nitrogen atoms, 0-1 sulfur atoms and 0-1 oxygen atoms.

Non-limiting examples of heterocycle and heteroaryl groups include pyridine, pyridimidine, pyrazine, morpholin-3-one, piperazine-2-one, pyridine-2-one, piperidine, morpholine, piperazine, isoxazole, isothiazole, pyrazole, imidazole, oxazole, thiazole, isoxazoline, pyrazoline, imidazoline, 1,2,3-triazole, 1,2,4-triazole, 1,3,4-oxadiazole, 1,3,4-thiadiazole, 1,2,4-oxadiazole, 1,2,4-thiadiazole, pyrazol-5-one, pyrrolidine-2,5-dione, imidazolidine-2,4-dione, pyrrolidine, pyrrole, furan, thiophene, and the like.

The term “heterocycloalkyl” refers to the group alkylene-heterocycle, wherein both heterocycle and alkylene are as defined above.

The above terms (e.g., “alkyl,” “alkoxy,” “aryl” and “heteroaryl”), in some embodiments, will include both substituted and unsubstituted forms of the indicated radical. Preferred substituents for each type of radical are provided below. For brevity, the terms aryl and heteroaryl will refer to substituted or unsubstituted versions as provided below.

Substituents for the “alkyl,” “alkoxy,” aryl and heteroaryl, etc. groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—, C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —N₃, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, C₁₋₈alkyl, C₃₋₆cycloalkyl, C₂₋₈alkenyl, C₂₋₈alkynyl, unsubstituted aryl and heteroaryl, (unsubstituted aryl)-C₁₋₄alkyl, and unsubstituted aryloxy-C₁₋₄alkyl. In some embodiments, the nitrogen atoms in the substituents are optionally quaternized. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆alkyl.

As used herein, the term “heteroatom” is meant to include oxygen (O), nitrogen (N), sulfur (S) and silicon (Si).

It is understood that the above definitions are not intended to include impermissible substitution patterns (e.g., methyl substituted with 5 fluoro groups). Such impermissible substitution patterns are well known to the skilled artisan.

The term “compound” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

The term “racemates” refers to a mixture of enantiomers.

The term “stereoisomer” or “stereoisomers” refer to compounds that differ in the chirality of one or more stereocenters. Stereoisomers include enantiomers and diastereomers.

The term “tautomer” refer to alternate forms of a compound that differ in the position of a proton, such as enol keto and imine enamine tautomers, or the tautomeric forms of heteroaryl groups containing a ring atom attached to both a ring NH moiety and a ring ═N moiety such as pyrazoles, imidazoles, benzimidazoles, triazoles, and tetrazoles.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge, S. M., et al, “Pharmaceutical Salts”, Journal of pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Accordingly, in one embodiment provided is a compound having Formula (I) or a pharmaceutically acceptable salt, ester, or prodrug thereof:

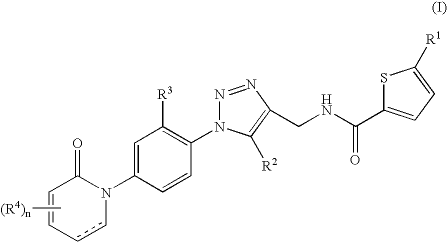

wherein
R¹ is halogen;
R² is hydrogen or halogen;
R³ is selected from the group consisting of —NO₂, —NR^{5a}R^{5b}, -L-NR^{5a}R^{5b}, —NHC(O)NR^{5a}R^{5b}, —NHC(O)R^{5c}, —NHC(O)Y, C₁₋₆ alkyl, —CO₂H, —C(O)NR^{5a}R^{5b}, —C(O)NR^{5a}Y, —C(O)NH-L-Y, —OH, C₁₋₆ alkoxy, —O-L-NR^{5a}R^{5b}, —O-L-O—C(O)NR^{5a}R^{5b}, —Y, —O—Y, —O-L-Y, —O-L-Y-L-Y, and —S(O)ₚR^{5c}, wherein said C₁₋₆ alkyl and C₁₋₆ alkoxy are optionally substituted with one to three substituents selected from R⁶;
R⁴ is independently selected from the group consisting of halogen, —OH, —O-L-Y, —O-L-NR^{5a}R^{5b}, and C₁₋₆ alkoxy optionally substituted with one to three substituents selected from R⁶;
L is C₁-C₄ alkylene;
Y is aryl, heteroaryl, or heterocyclic ring, wherein said aryl and heteroaryl are optionally substituted with one to three R⁶ and said heterocyclic ring is optionally substituted with oxo and optionally with one to three R⁶ or R⁸;
R^{5a} and R^{5b} are independently hydrogen or C₁₋₈ alkyl optionally substituted with one to three R⁶, or R^{5a} and R^{5b} together with the nitrogen atom to which they are both attached to form a 5 to 7 membered heterocyclic ring optionally having one additional ring heteroatom selected from N, NR⁶, O, or S(O)ₚ and where said ring is optionally substituted with one to three substituents selected from R⁶;
R^{5c} is C₁₋₈ alkyl optionally substituted with one to three R⁶;
R⁶ is independently selected from the group consisting of halogen, —OH, —R⁷, —OR⁷, oxo, —SR⁷, —S(O)R⁷, —S(O)₂R⁷, —SO₂NH₂, —C(O)NH₂, —C(O)R⁷, —C(NH)R⁷, —NHC(O)R⁷, —NHC(NH)R⁷, —NHC(O)NH₂, —CO₂H, —NH₂, —NHR⁷, —N(R⁷)₂;
R⁷ is independently C₁₋₆ alkyl;
R⁸ is -L-heteroaryl optionally substituted with one to three substituents selected from R⁶;
   n is 0, 1, or 2;
   p is 0, 1, or 2; and
the dashed lines [symbol] are independently single or double bonds;
provided that R³ is not

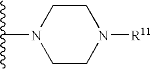

where R¹¹ is hydrogen or alkyl.

In another embodiment provided is a compound having Formula (II) or a pharmaceutically acceptable salt, ester, or prodrug thereof:

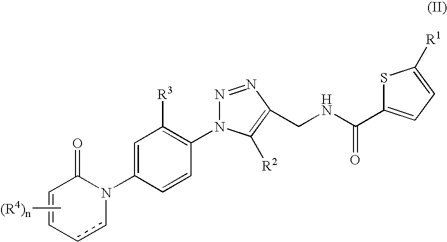

wherein
R¹ is halogen;
R² is hydrogen or halogen;
R³ is selected from the group consisting of —NO₂, —NR^{5a}R^{5b}, -L-NR^{5a}R^{5b}, —NHC(O)NR^{5a}R^{5b}, —NHC(O)R^{5c}, —NHC(O)Y, C₁₋₆ alkyl, —CO₂H, —C(O)NR^{5a}R^{5b}, —C(O)NH-L-Y, OH, C₁₋₆ alkoxy, —O-L-NR^{5a}R^{5b}, —O-L-O—C(O)NR^{5a}R^{5b}, —Y, —O—Y, —O-L-Y, —O-L-Y-L-Y, and —S(O)ₚR^{5c}, wherein said C₁₋₆ alkyl and C₁₋₆ alkoxy are optionally substituted with one to three substituents selected from R⁶;
R⁴ is independently selected from the group consisting of halo, OH, —O-L-Y, —O-L-NR^{5a}R^{5b}, and C₁₋₆ alkoxy optionally substituted with one to three substituents selected from R⁶;
L is C₁-C₄ alkylene;
Y is phenyl, heteroaryl, or heterocyclic ring, wherein said phenyl and heteroaryl are optionally substituted with one to three R⁶ and said heterocyclic ring is optionally substituted with oxo and optionally with one to three R⁶ or R⁸;
R^{5a} and R^{5b} are independently hydrogen or C₁₋₈ alkyl optionally substituted with one to three R⁶, or R^{5a} and R^{5b} together with the nitrogen atom to which they are both attached to form a 5 to 7 membered heterocyclic ring optionally having one additional ring heteroatom selected from N, NR⁶, O, or S(O)ₚ and where said ring is optionally substituted with one to three substituents selected from R⁶;
R^{5c} is C₁₋₈ alkyl optionally substituted with one to three R⁶;
R⁶ is independently selected from the group consisting of halogen, —OH, —R⁷, —OR⁷, oxo, —SR⁷, —S(O)R⁷, —S(O)₂R⁷, —SO₂NH₂, —C(O)NH₂, —C(O)R⁷, —C(NH)R⁷, —NHC(O)R⁷, —NHC(NH)R⁷, —NHC(O)NH₂, —CO₂H, —NH₂, —NHR⁷, —N(R⁷)₂;
R⁷ is independently C₁₋₆ alkyl;
R⁸ is -L-heteroaryl optionally substituted with one to three substituents selected from R⁶;
   n is 0, 1, or 2;
   p is 0, 1, or 2; and
dashed lines [symbol] are independently single or double bonds;
provided that R³ is not

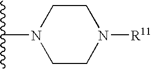

where R¹¹ is hydrogen or alkyl.

As used herein, the wavy line (“ [symbol] ”) indicates the point of attachment to the rest of the molecule.

In one embodiment, provided is a compound of Formula (Ia) or a pharmaceutically acceptable salt, ester, or prodrug thereof:

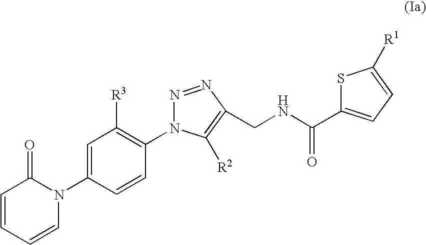

wherein R¹, R², and R³ are as previously defined for Formula (I).

In one embodiment, provided is a compound of Formula (Ib) or a pharmaceutically acceptable salt, ester, or prodrug thereof:

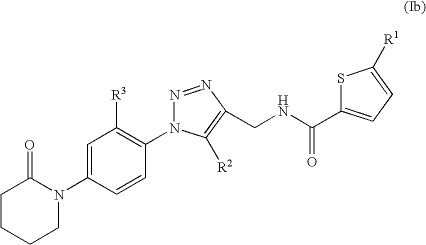

wherein R¹, R², and R³ are as previously defined for Formula (I).

It is contemplated that compounds of Formulas (I), (II), (Ia) and (Ib) will consistently provide highly active Factor Xa inhibitors.

In some embodiments of the compounds of Formulas (I), (II), (Ia), and (Ib), R¹ is chlorine.

In some embodiments of Formulas (I), (II), (Ia), and (Ib), R² is hydrogen.

In some embodiments of Formulas (I), (II), (Ia), and (Ib), R³ is selected from the group consisting of —NO₂, —NR^{5a}R^{5b}, -L-NR^{5a}R^{5b}, —NHC(O)NR^{5a}R^{5b}, NHC(O)R^{5c}, —NHC(O)Y, C₁₋₆ alkyl, —CO₂H, C(O)NR^{5a}R^{5b}, —C(O)NH-L-Y, —OH, C₁₋₆ alkoxy, —O-L-NR^{5a}R^{5b}, —O-L-O—C(O)NR^{5a}R^{5b}, —Y, —O—Y, —O-L-Y, —O-L-Y-L-Y, and —S(O)ₚR^{5c}, wherein said C₁₋₆ alkyl and C₁₋₆ alkoxy are optionally substituted with one to three substituents selected from R⁶.

In some embodiments, R⁶ is independently selected from the group consisting of halogen, —OH, —R⁷, —OR⁷, —SR⁷, —S(O)R⁷, —S(O)₂R⁷, —SO₂NH₂, —C(O)NH₂, —C(O)R⁷, —C(NH)R⁷, —NHC(O)R⁷, —NHC(NH)R⁷, —NHC(O)NH₂, —CO₂H, —NH₂, —NHR⁷, and —N(R⁷)₂. In some embodiments, R⁶ is attached to a carbon atom. In some embodiments, R⁶ is attached to a nitrogen atom and is independently selected from the group consisting of —OH, —R⁷, —OR⁷, —S(O)₂R⁷, —SO₂NH₂, —C(O)NH₂, —C(O)R⁷, —C(NH)R⁷, —NHC(O)R⁷, —NHC(NH)R⁷, —NHC(O)NH₂, —NH₂, —NHR⁷, and —N(R⁷)₂. In some embodiments, R⁶ is attached to a nitrogen atom and is independently selected from the group consisting of —OH, —R⁷, —OR⁷, —S(O)₂R⁷, —SO₂NH₂, —C(O)NH₂, —C(O)R⁷, and —C(NH)R⁷.

In some embodiments, R³ is attached to the phenyl ring through a nitrogen atom and is —NO₂, —NR^{5a}R^{5b}, —NHC(O)NR^{5a}R^{5b}, —NHC(O)R^{5c}, or —NHC(O)Y. In some aspects, R³ is selected from a group consisting of

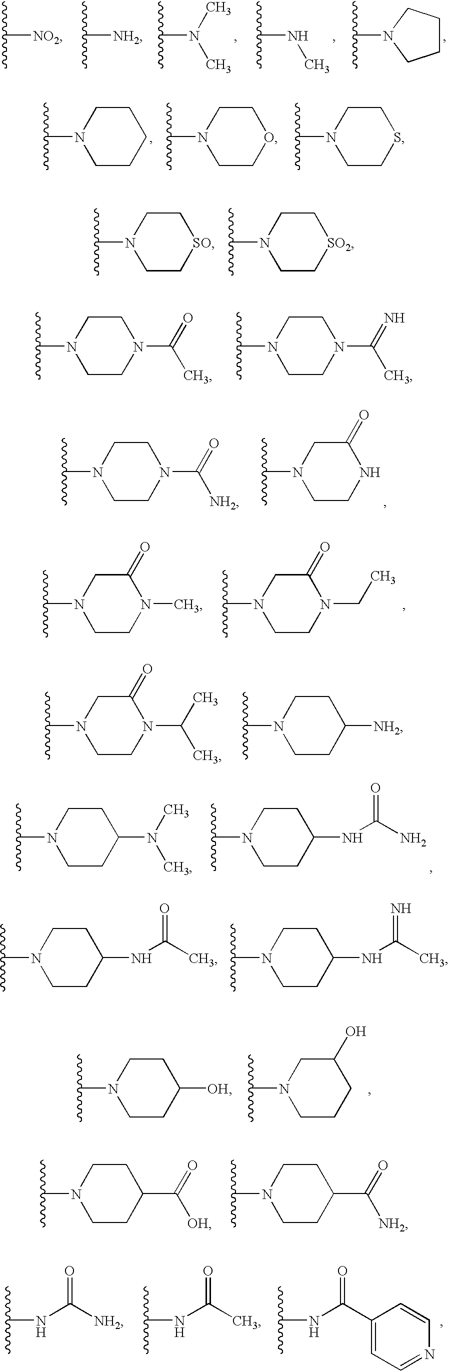

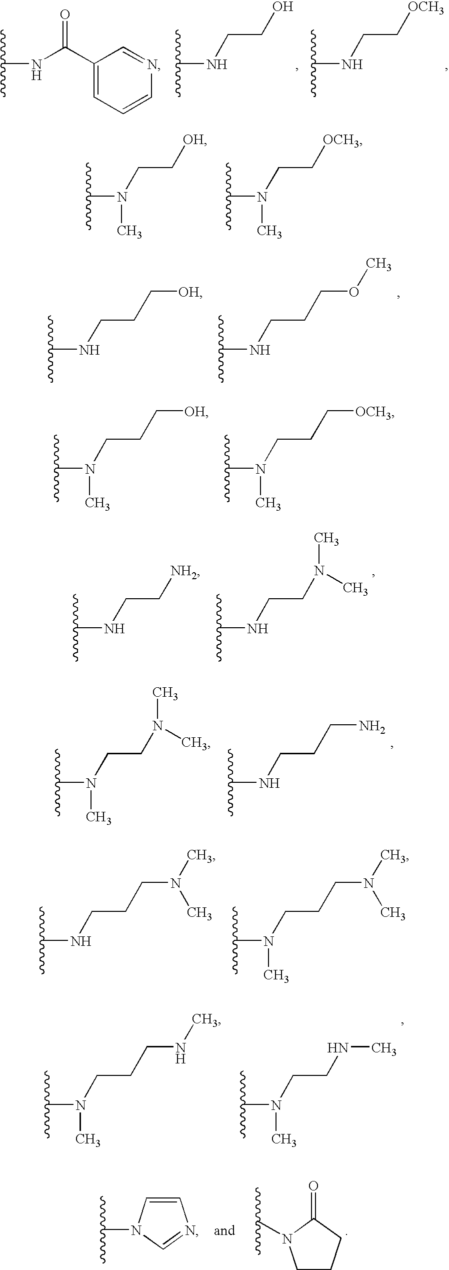

In other embodiments, R³ is optionally substituted aryl or heteroaryl. In some aspects, R³ is selected from a group consisting of

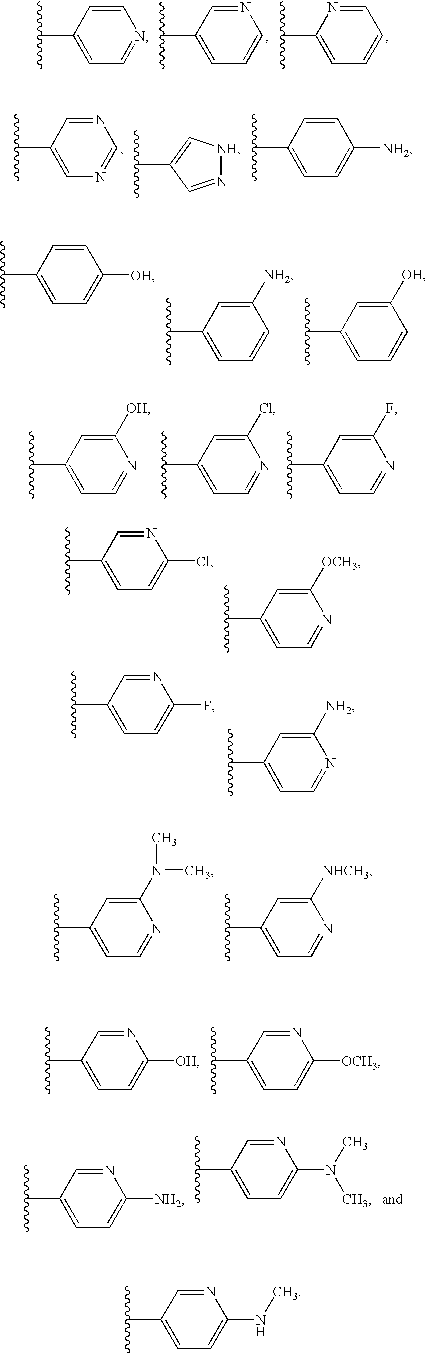

In other embodiments, R³ is attached to the phenyl ring through a carbon atom and is -L-NR^{5a}R^{5b}, —CO₂H, —C(O)NR^{5a}R^{5b}, or —C(O)NH-L-Y. In some embodiments, R³ is optionally substituted C₁₋₆ alkyl. In some embodiments, R³ is —C(O)NR^{5a}Y. In some aspects, R³ is selected from a group consisting of

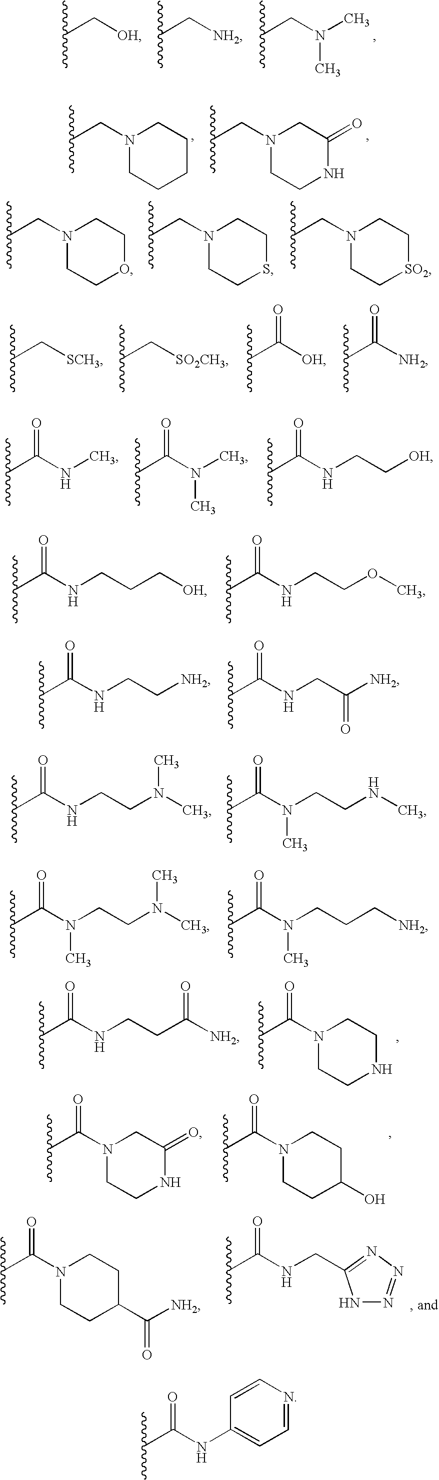

In other embodiments, R³ is attached to the phenyl ring through an oxygen atom and is optionally substituted C₁₋₆ alkoxy, —O-L-NR^{5a}R⁵, —O-L-O—C(O)NR^{5a}R^{5b}, —O—Y, or —O-L-Y. In some embodiments, R³ is —OH. In some embodiments, R³ is —O-L-Y-L-Y. In some aspects, R³ is selected from a group consisting of

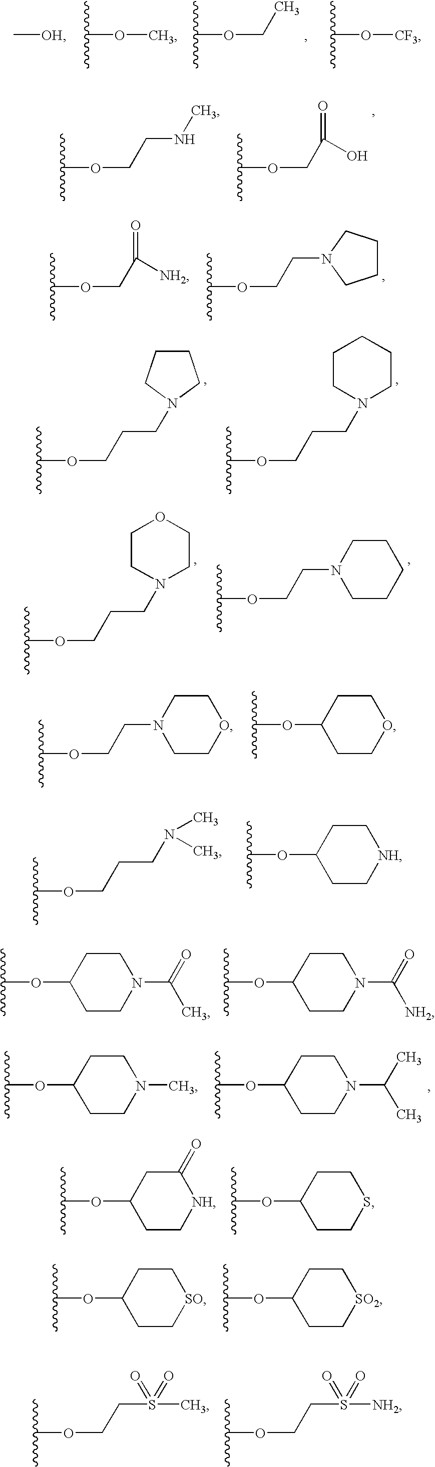

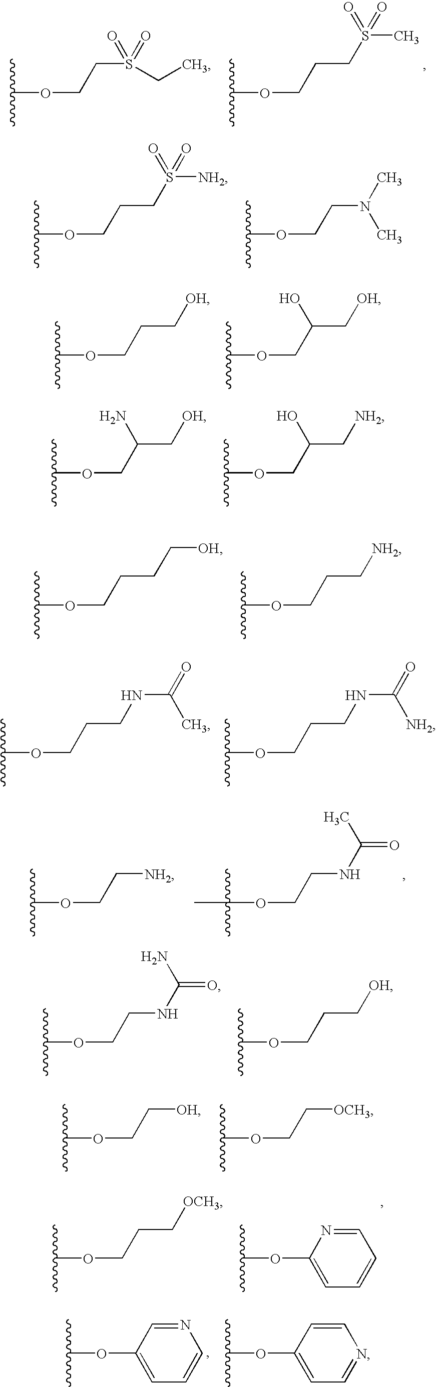

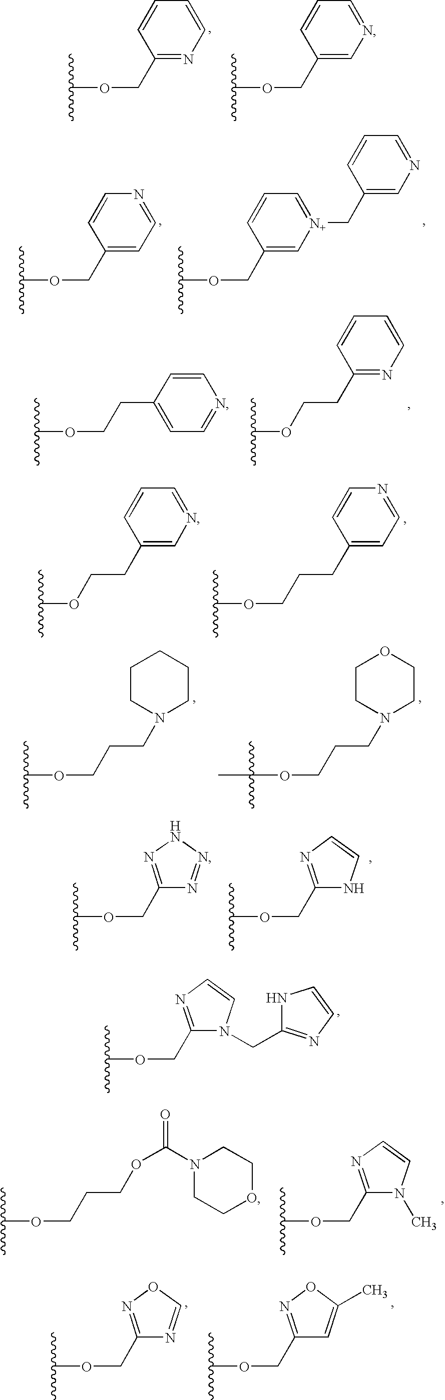

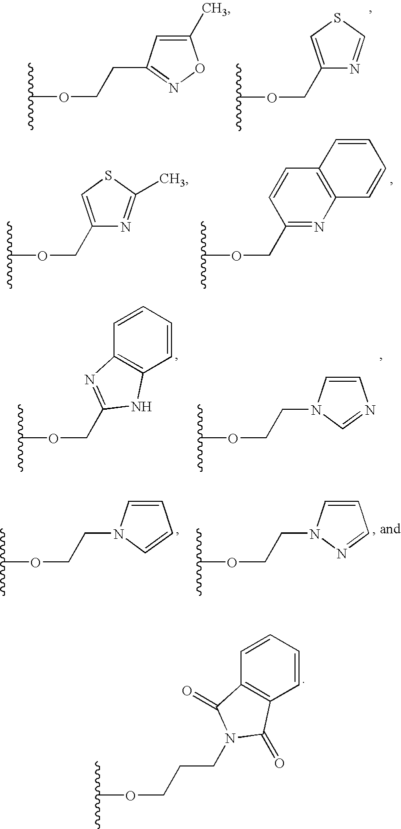

In other embodiments, R³ is attached to the phenyl ring through a sulfur atom and is —S(O)ₚR^{5c}. In some aspects, R³ is selected from a group consisting of

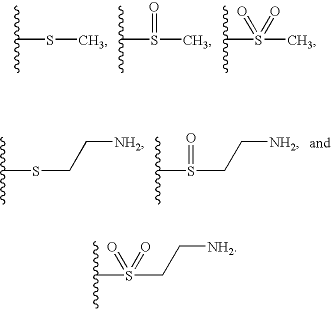

In some aspects of the compounds or compositions of the present invention and subject to the provisos recited herein, provided is a compound, stereoisomer, or a pharmaceutically acceptable salt thereof selected from Table 1.

TABLE 1

| Cmpd | Structure | Name |
|---|---|---|
| 1 | 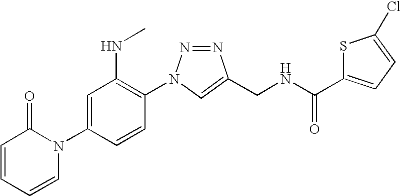 | 5-Chloro-N-((1-(2-(methylamino)-4-(2-oxopyridin-1(2H)-yl)phenyl)-1H-1,2,3-triazol-4-yl)methyl)thiophene-2-carboxamide |
| 2 | 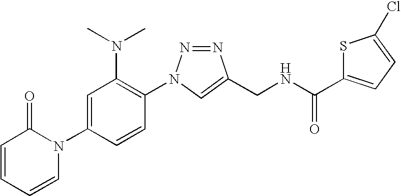 | 5-Chloro-N-((1-(2-(dimethylamino)-4-(2-oxopyridin-1(2H)-yl)phenyl)-1H-1,2,3-triazol-4-yl)methyl)thiophene-2-carboxamide |
| 3 | 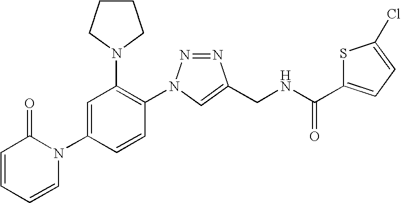 | 5-Chloro-N-((1-(4-(2-oxopyridin-1(2H)-yl)-2-(pyrrolidin-1-yl)phenyl)-1H-1,2,3-triazol-4-yl)methyl)thiophene-2-carboxamide |
| 4 | 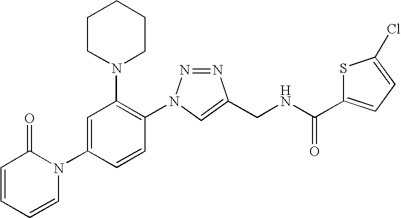 | 5-Chloro-N-((1-(4-(2-oxopyridin-1(2H)-yl)-2-(piperidin-1-yl)phenyl)-1H-1,2,3-triazol-4-yl)methyl)thiophene-2-carboxamide |
| 5 | 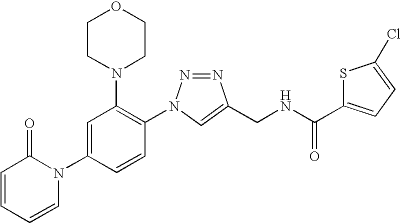 | 5-Chloro-N-((1-(2-morpholino-4-(2-oxopyridin-1(2H)-yl)phenyl)-1H-1,2,3-triazol-4-yl)methyl)thiophene-2-carboxamide |
| 6 | 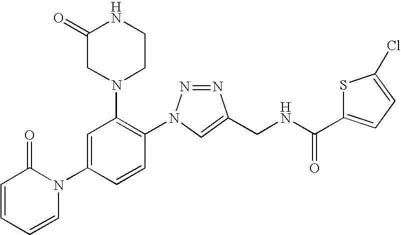 | 5-Chloro-N-((1-(2-(3-oxopiperazin-1-yl)-4-(2-oxopyridin-1(2H)-yl)phenyl)-1H-1,2,3-triazol-4-yl)methyl)thiophene-2-carboxamide |
| 7 | 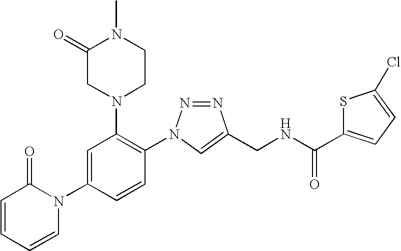 | 5-Chloro-N-((1-(2-(4-methyl-3-oxopiperazin-1-yl)-4-(2-oxopyridin-1(2H)-yl)phenyl)-1H-1,2,3-triazol-4-yl)methyl)thiophene-2-carboxamide |
| 8 | 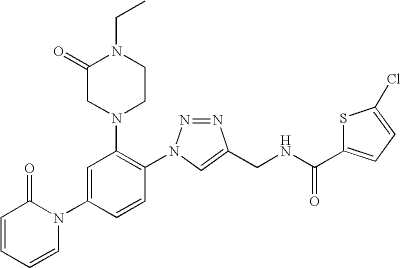 | 5-Chloro-N-((1-(2-(4-ethyl-3-oxopiperazin-1-yl)-4-(2-oxopyridin-1(2H)-yl)phenyl)-1H-1,2,3-triazol-4-yl)methyl)thiophene-2-carboxamide |
| 9 | 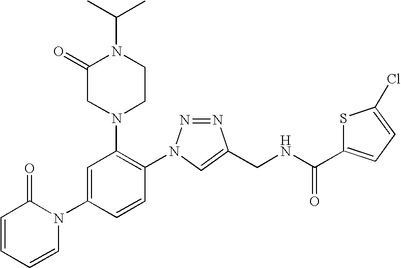 | 5-Chloro-N-((1-(2-(4-isopropyl-3-oxopiperazin-1-yl)-4-(2-oxopyridin-1(2H)-yl)phenyl)-1H-1,2,3-triazol-4-yl)methyl)thiophene-2-carboxamide |
| 10 | 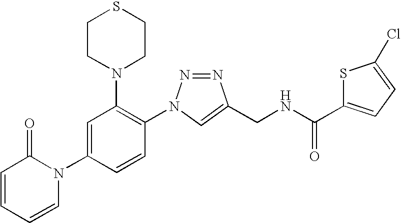 | 5-Chloro-N-((1-(4-(2-oxopyridin-1(2H)-yl)-2-thiomorpholinophenyl)-1H-1,2,3-triazol-4-yl)methyl)thiophene-2-carboxamide |
| 11 | 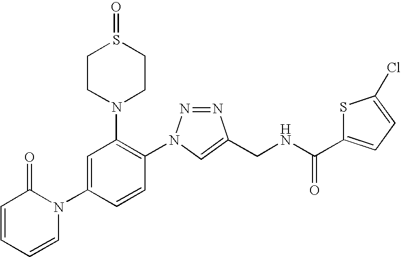 | 5-Chloro-N-((1-(4-(2-oxopyridin-1(2H)-yl)-2-((1-oxo-)thio-morpholino)phenyl)-1H-1,2,3-triazol-4-yl)methyl)thiophene-2-carboxamide |
| 12 | 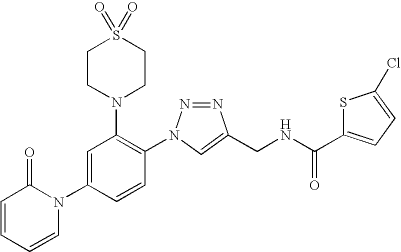 | 5-Chloro-N-((1-(4-(2-oxopyridin-1(2H)-yl)-2-((1,1-dioxo-)thio-morpholino)phenyl)-1H-1,2,3-triazol-4-yl)methyl)thiophene-2-carboxamide |
| 13 | 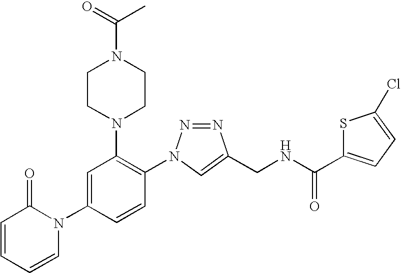 | N-((1-(2-(4-Acetylpiperazin-1-yl)-4-(2-oxopyridin-1(2H)-yl)phenyl)-1H-1,2,3-triazol-4-yl)methyl)-5-chlorothiophene-2-carboxamide |
| 14 | 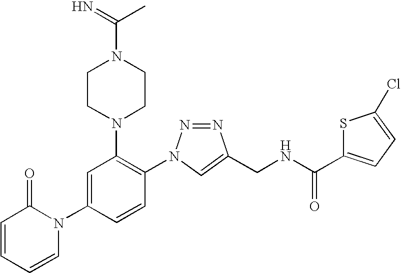 | 5-Chloro-N-((1-(2-(4-(1-iminoethyl)piperazin-1-yl)-4-(2-oxopyridin-1(2H)-yl)phenyl)-1H-1,2,3-triazol-4-yl)methyl)thiophene-2-carboxamide |
| 15 | 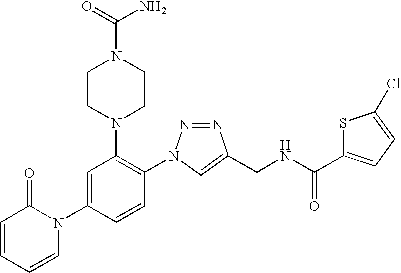 | 4-(2-(4-((2-Chlorothiophene-5-carboxamido)methyl)-1H-1,2,3-triazol-1-yl)-5-(2-oxopyridin-1(2H)-yl)phenyl)piperazine-1-carboxamide |
| 16 | 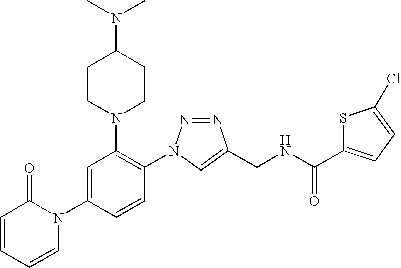 | 5-Chloro-N-((1-(2-(4-(dimethylamino)piperidin-1-yl)-4-(2-oxopyridin-1(2H)-yl)phenyl)-1H-1,2,3-triazol-4-yl)methyl)thiophene-2-carboxamide |
| 17 | 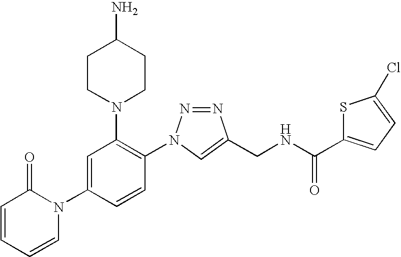 | N-((1-(2-(4-Aminopiperidin-1-yl)-4-(2-oxopyridin-1(2H)-yl)phenyl)-1H-1,2,3-triazol-4-yl)methyl)-5-chlorothiophene-2-carboxamide |
| 18 | 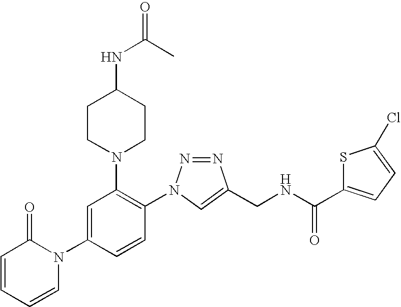 | N-((1-(2-(4-Acetamidopiperidin-1-yl)-4-(2-oxopyridin-1(2H)-yl)phenyl)-1H-1,2,3-triazol-4-yl)methyl)-5-chlorothiophene-2-carboxamide |
| 19 | 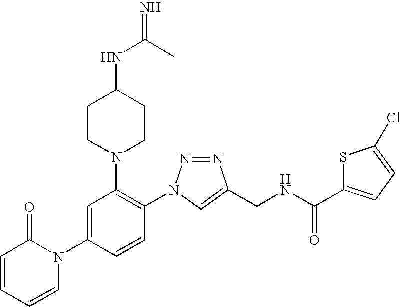 | N-((1-(2-(4-Acetamidinopiperidin-1-yl)-4-(2-oxopyridin-1(2H)-yl)phenyl)-1H-1,2,3-triazol-4-yl)methyl)-5-chlorothiophene-2-carboxamide |
| 20 | 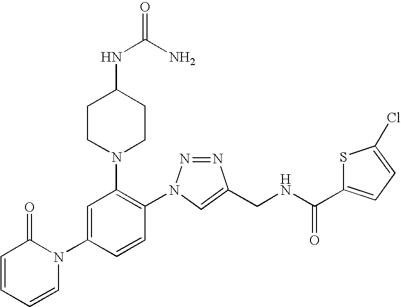 | 1-(1-(2-(4-((2-Chlorothiophene-5-carboxamido)methyl)-1H-1,2,3-triazol-1-yl)-5-(2-oxopyridin-1(2H)-yl)phenyl)piperidin-4-yl)urea |
| 21 | 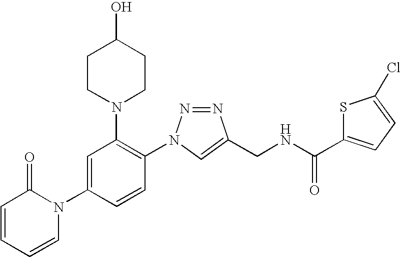 | 5-Chloro-N-((1-(2-(4-hydroxypiperidin-1-yl)-4-(2-oxopyridin-1(2H)-yl)phenyl)-1H-1,2,3-triazol-4-yl)methyl)thiophene-2-carboxamide |
| 22 | 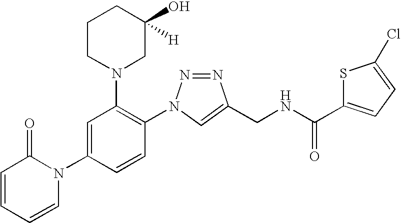 | 5-Chloro-N-((1-(2-((R)-3-hydroxypiperidin-1-yl)-4-(2-oxopyridin-1(2H)-yl)phenyl)-1H-1,2,3-triazol-4-yl)methyl)thiophene-2-carboxamide |
| 23 | 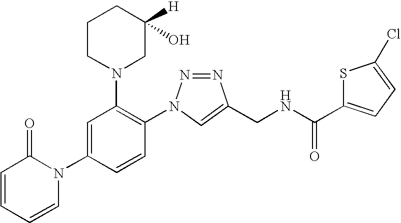 | 5-Chloro-N-((1-(2-((S)-3-hydroxypiperidin-1-yl)-4-(2-oxopyridin-1(2H)-yl)phenyl)-1H-1,2,3-triazol-4-yl)methyl)thiophene-2-carboxamide |
| 24 | 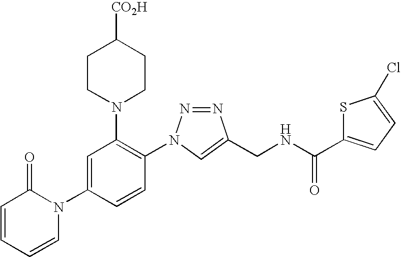 | 1-(2-(4-((2-Chlorothiophene-5-carboxamido)methyl)-1H-1,2,3-triazol-1-yl)-5-(2-oxopyridin-1(2H)-yl)phenyl)piperidine-4-carboxylicacid |
| 25 | 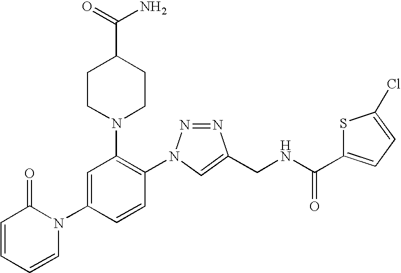 | 1-(2-(4-((2-Chlorothiophene-5-carboxamido)methyl)-1H-1,2,3-triazol-1-yl)-5-(2-oxopyridin-1(2H)-yl)phenyl)piperidine-4-carboxamide |
| 26 | 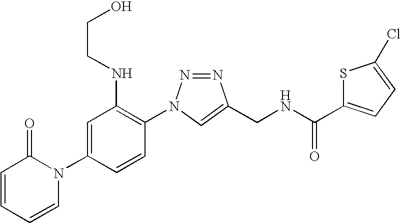 | 5-Chloro-N-((1-(2-(2-hydroxyethylamino)-4-(2-oxopyridin-1(2H)-yl)phenyl)-1H-1,2,3-triazol-4-yl)methyl)thiophene-2-carboxamide |
| 27 | 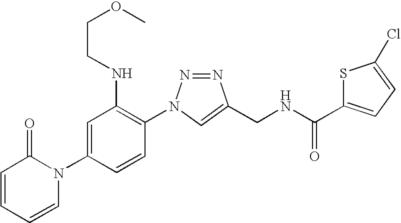 | 5-Chloro-N-((1-(2-(2-methoxyethylamino)-4-(2-oxopyridin-1(2H)-yl)phenyl)-1H-1,2,3-triazol-4-yl)methyl)thiophene-2-carboxamide |
| 28 | 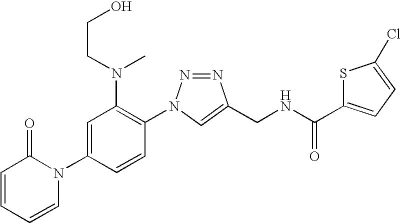 | 5-Chloro-N-((1-(2-((2-hydroxyethyl)(methyl)amino)-4-(2-oxopyridin-1(2H)-yl)phenyl)-1H-1,2,3-triazol-4-yl)methyl)thiophene-2-carboxamide |
| 29 | 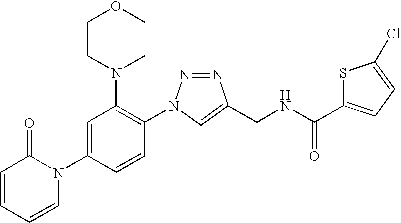 | 5-Chloro-N-((1-(2-((2-methoxyethyl)(methyl)amino)-4-(2-oxopyridin-1(2H)-yl)phenyl)-1H-1,2,3-triazol-4-yl)methyl)thiophene-2-carboxamide |
| 30 | 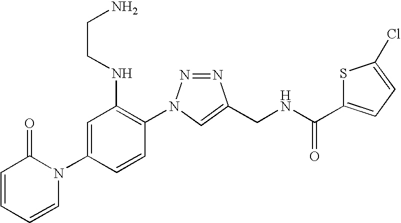 | N-((1-(2-(2-Aminoethylamino)-4-(2-oxopyridin-1(2H)-yl)phenyl)-1H-1,2,3-triazol-4-yl)methyl)-5-chlorothiophene-2-carboxamide |
| 31 | 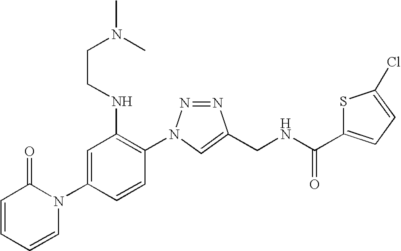 | 5-Chloro-N-((1-(2-(2-(dimethylamino)ethylamino)-4-(2-oxopyridin-1(2H)-yl)phenyl)-1H-1,2,3-triazol-4-yl)methyl)thiophene-2-carboxamide |
| 32 | 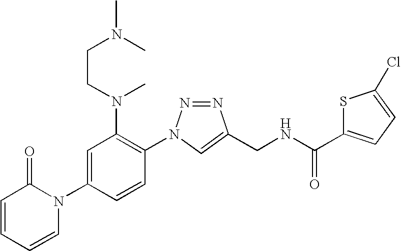 | 5-Chloro-N-((1-(2-((2-(dimethylamino)ethyl)(methyl)amino)-4-(2-oxopyridin-1(2H)-yl)phenyl)-1H-1,2,3-triazol-4-yl)methyl)thiophene-2-carboxamide |
| 33 | 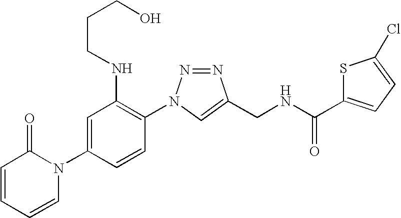 | 5-Chloro-N-((1-(2-(3-hydroxypropylamino)-4-(2-oxopyridin-1(2H)-yl)phenyl)-1H-1,2,3-triazol-4-yl)methyl)thiophene-2-carboxamide |
| 34 | 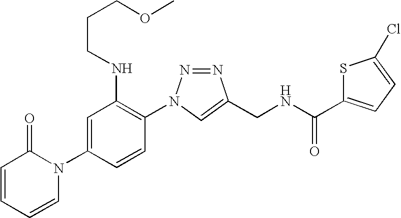 | 5-Chloro-N-((1-(2-(3-methoxypropylamino)-4-(2-oxopyridin-1(2H)-yl)phenyl)-1H-1,2,3-triazol-4-yl)methyl)thiophene-2-carboxamide |
| 35 | 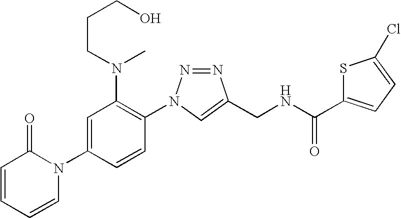 | 5-Chloro-N-((1-(2-((3-hydroxypropyl)(methyl)amino)-4-(2-oxopyridin-1(2H)-yl)phenyl)-1H-1,2,3-triazol-4-yl)methyl)thiophene-2-carboxamide |
| 36 | 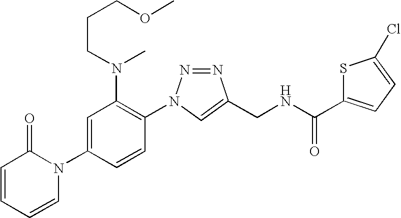 | 5-Chloro-N-((1-(2-((3-methoxypropyl)(methyl)amino)-4-(2-oxopyridin-1(2H)-yl)phenyl)-1H-1,2,3-triazol-4-yl)methyl)thiophene-2-carboxamide |
| 37 | 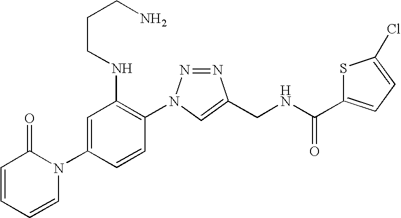 | N-((1-(2-(3-Aminopropylamino)-4-(2-oxopyridin-1(2H)-yl)phenyl)-1H-1,2,3-triazol-4-yl)methyl)-5-chlorothiophene-2-carboxamide |
| 38 | 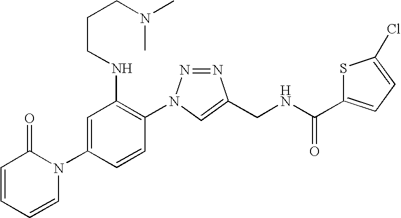 | 5-Chloro-N-((1-(2-(3-(dimethylamino)propylamino)-4-(2-oxopyridin-1(2H)-yl)phenyl)-1H-1,2,3-triazol-4-yl)methyl)thiophene-2-carboxamide |
| 39 | 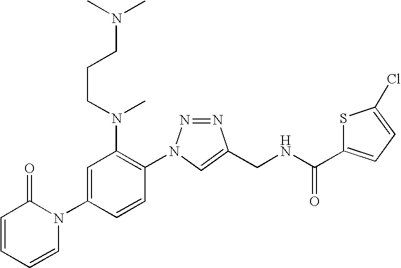 | 5-Chloro-N-((1-(2-((3-(dimethylamino)propyl)(methyl)amino)-4-(2-oxopyridin-1(2H)-yl)phenyl)-1H-1,2,3-triazol-4-yl)methyl)thiophene-2-carboxamide |
| 40 | 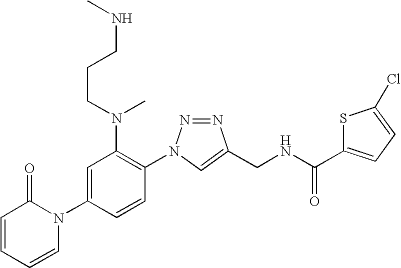 | 5-Chloro-N-((1-(2-(methyl(3-(methylamino)propyl)amino)-4-(2-oxopyridin-1(2H)-yl)phenyl)-1H-1,2,3-triazol-4-yl)methyl)thiophene-2-carboxamide |
| 41 | 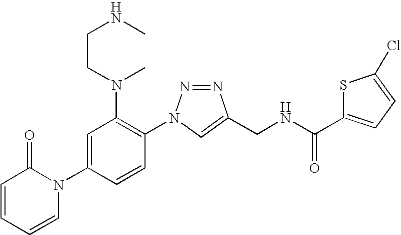 | 5-Chloro-N-((1-(2-(methyl(2-(methylamino)ethyl)amino)-4-(2-oxopyridin-1(2H)-yl)phenyl)-1H-1,2,3-triazol-4-yl)methyl)thiophene-2-carboxamide |
| 42 | 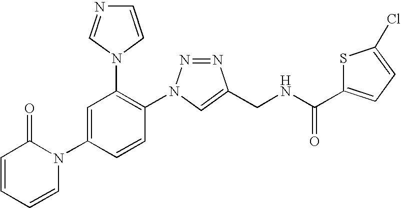 | N-((1-(2-(1H-Imidazol-1-yl)-4-(2-oxopyridin-1(2H)-yl)phenyl)-1H-1,2,3-triazol-4-yl)methyl)-5-chlorothiophene-2-carboxamide |
| 43 | 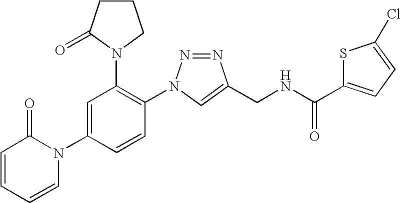 | 5-Chloro-N-((1-(4-(2-oxopyridin-1(2H)-yl)-2-(2-oxopyrrolidin-1-yl)phenyl)-1H-1,2,3-triazol-4-yl)methyl)thiophene-2-carboxamide |
| 44 | 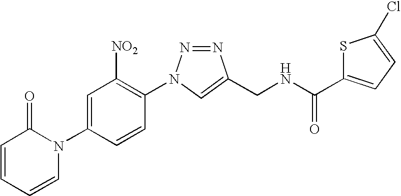 | 5-Chloro-N-((1-(2-nitro-4-(2-oxopyridin-1(2H)-yl)phenyl)-1H-1,2,3-triazol-4-yl)methyl)thiophene-2-carboxamide |
| 45 | 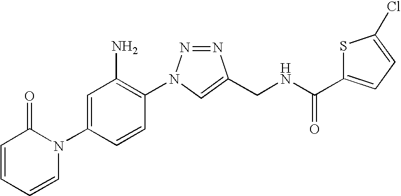 | N-((1-(2-Amino-4-(2-oxopyridin-1(2H)-yl)phenyl)-1H-1,2,3-triazol-4-yl)methyl)-5-chlorothiophene-2-carboxamide |
| 46 | 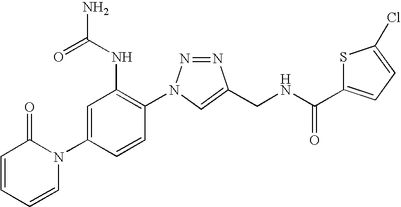 | 1-(2-(4-((2-Chlorothiophene-5-carboxamido)methyl)-1H-1,2,3-triazol-1-yl)-5-(2-oxopyridin-1(2H)-yl)phenyl)urea |
| 47 | 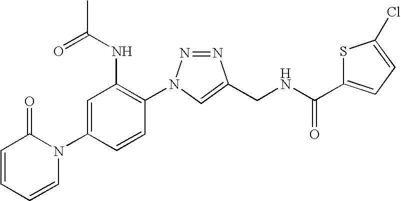 | N-((1-(2-Acetamido-4-(2-oxopyridin-1(2H)-yl)phenyl)-1H-1,2,3-triazol-4-yl)methyl)-5-chlorothiophene-2-carboxamide |
| 48 | 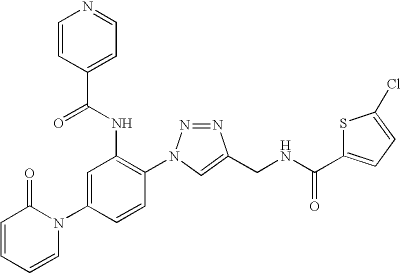 | N-(2-(4-((2-Chlorothiophene-5-carboxamido)methyl)-1H-1,2,3-triazol-1-yl)-5-(2-oxopyridin-1(2H)-yl)phenyl)isonicotinamide |
| 49 | 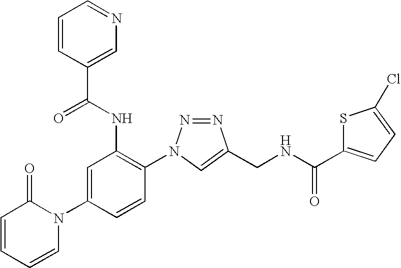 | N-(2-(4-((2-Chlorothiophene-5-carboxamido)methyl)-1H-1,2,3-triazol-1-yl)-5-(2-oxopyridin-1(2H)-yl)phenyl)nicotinamide |
| 50 | 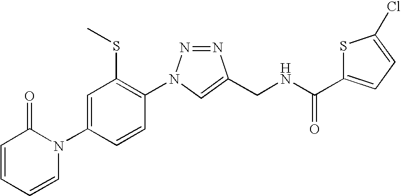 | 5-Chloro-N-((1-(2-(methylthio)-4-(2-oxopyridin-1(2H)-yl)phenyl)-1H-1,2,3-triazol-4-yl)methyl)thiophene-2-carboxamide |
| 51 | 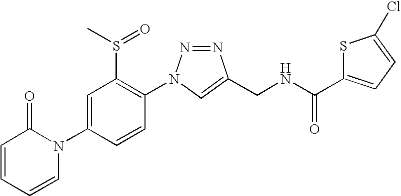 | 5-Chloro-N-((1-(2-(methylsulfoxy)-4-(2-oxopyridin-1(2H)-yl)phenyl)-1H-1,2,3-triazol-4-yl)methyl)thiophene-2-carboxamide |
| 52 | 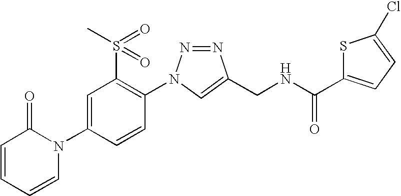 | 5-Chloro-N-((1-(2-(methylsulfonyl)-4-(2-oxopyridin-1(2H)-yl)phenyl)-1H-1,2,3-triazol-4-yl)methyl)thiophene-2-carboxamide |
| 53 | 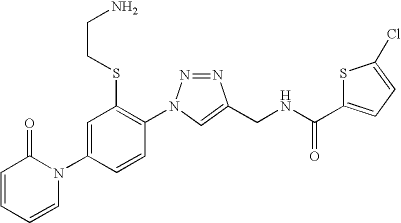 | N-((1-(2-(2-Aminoethylthio)-4-(2-oxopyridin-1(2H)-yl)phenyl)-1H-1,2,3-triazol-4-yl)methyl)-5-chlorothiophene-2-carboxamide |
| 54 | 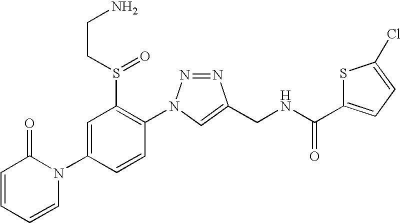 | N-((1-(2-(2-Aminoethylsulfoxy)-4-(2-oxopyridin-1(2H)-yl)phenyl)-1H-1,2,3-triazol-4-yl)methyl)-5-chlorothiophene-2-carboxamide |
| 55 | 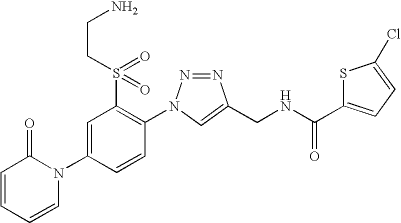 | N-((1-(2-(2-Aminoethylsulfonyl)-4-(2-oxopyridin-1(2H)-yl)phenyl)-1H-1,2,3-triazol-4-yl)methyl)-5-chlorothiophene-2-carboxamide |
| 56 | 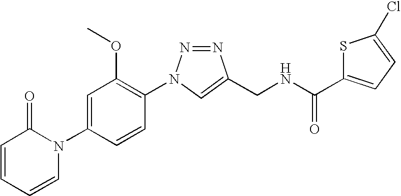 | 5-Chloro-N-((1-(2-methoxy-4-(2-oxopyridin-1(2H)-yl)phenyl)-1H-1,2,3-triazol-4-yl)methyl)thiophene-2-carboxamide |
| 57 | 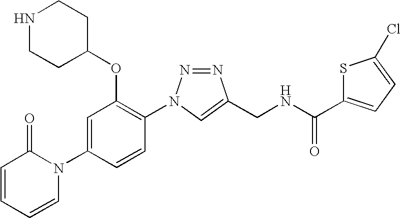 | 5-Chloro-N-((1-(4-(2-oxopyridin-1(2H)-yl)-2-(piperidin-4-yloxy)phenyl)-1H-1,2,3-triazol-4-yl)methyl)thiophene-2-carboxamide |
| 58 | 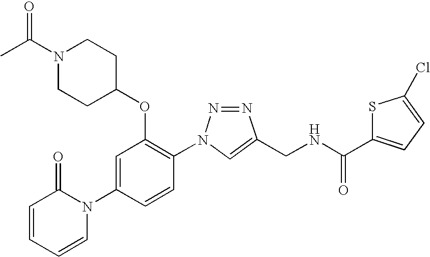 | N-((1-(2-(1-Acetylpiperidin-4-yloxy)-4-(2-oxopyridin-1(2H)-yl)phenyl)-1H-1,2,3-triazol-4-yl)methyl)-5-chlorothiophene-2-carboxamide |
| 59 | 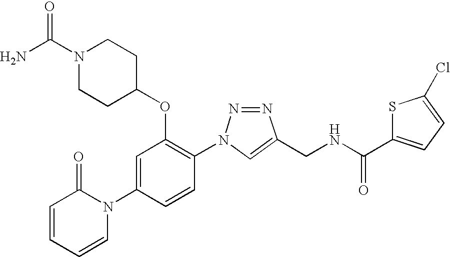 | 4-(2-(4-((2-Chlorothiophene-5-carboxamido)methyl)-1H-1,2,3-triazol-1-yl)-5-(2-oxopyridin-1(2H)-yl)phenoxy)piperidine-1-carboxamide |
| 60 | 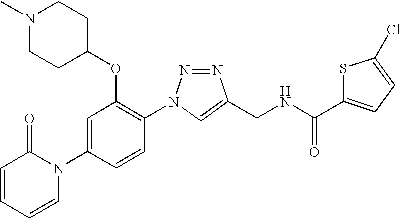 | 5-Chloro-N-((1-(2-(1-methylpiperidin-4-yloxy)-4-(2-oxopyridin-1(2H)-yl)phenyl)-1H-1,2,3-triazol-4-yl)methyl)thiophene-2-carboxamide |
| 61 | 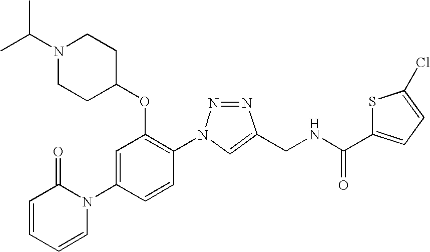 | 5-Chloro-N-((1-(2-(1-isopropylpiperidin-4-yloxy)-4-(2-oxopyridin-1(2H)-yl)phenyl)-1H-1,2,3-triazol-4-yl)methyl)thiophene-2-carboxamide |
| 62 | 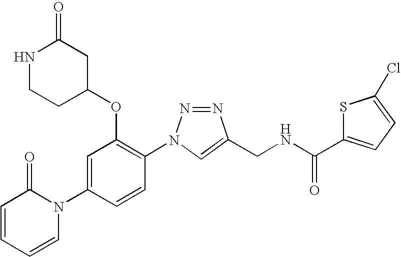 | 5-Chloro-N-((1-(2-(2-oxopiperidin-4-yloxy)-4-(2-oxopyridin-1(2H)-yl)phenyl)-1H-1,2,3-triazol-4-yl)methyl)thiophene-2-carboxamide |
| 63 | 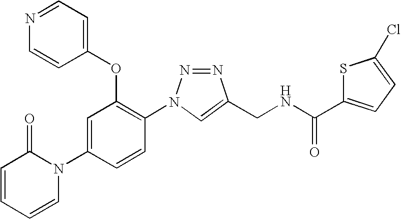 | 5-Chloro-N-((1-(4-(2-oxopyridin-1(2H)-yl)-2-(pyridin-4-yloxy)phenyl)-1H-1,2,3-triazol-4-yl)methyl)thiophene-2-carboxamide |
| 64 | 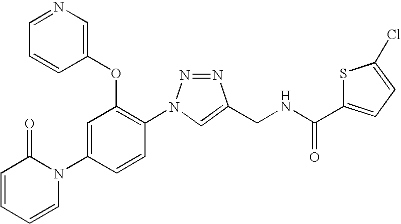 | 5-Chloro-N-((1-(4-(2-oxopyridin-1(2H)-yl)-2-(pyridin-3-yloxy)phenyl)-1H-1,2,3-triazol-4-yl)methyl)thiophene-2-carboxamide |
| 65 | 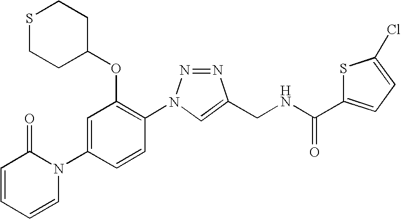 | 5-Chloro-N-((1-(4-(2-oxopyridin-1(2H)-yl)-2-(tetrahydro-2H-thiopyran-4-yloxy)phenyl)-1H-1,2,3-triazol-4-yl)methyl)thiophene-2-carboxamide |
| 66 | 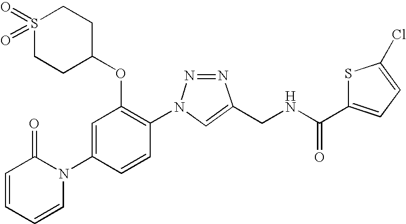 | 5-Chloro-N-((1-(4-(2-oxopyridin-1(2H)-yl)-2-(1,1-dioxo-tetrahydro-2H-thiopyran-4-yloxy)phenyl)-1H-1,2,3-triazol-4-yl)methyl)thiophene-2-carboxamide |
| 67 | 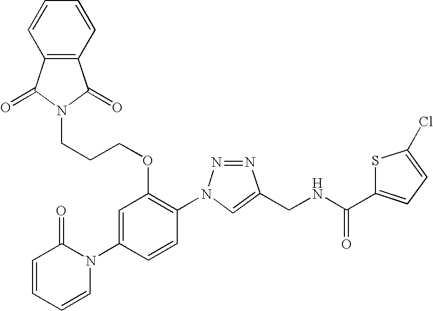 | 5-Chloro-N-((1-(2-(3-(1,3-dioxoisoindolin-2-yl)propoxy)-4-(2-oxopyridin-1(2H)-yl)phenyl)-1H-1,2,3-triazol-4-yl)methyl)thiophene-2-carboxamide |
| 68 | 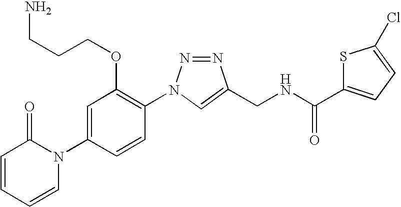 | N-((1-(2-(3-Aminopropoxy)-4-(2-oxopyridin-1(2H)-yl)phenyl)-1H-1,2,3-triazol-4-yl)methyl)-5-chlorothiophene-2-carboxamide |
| 69 | 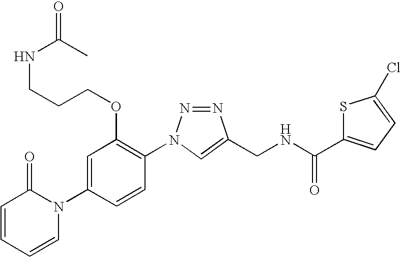 | N-((1-(2-(3-Acetamidopropoxy)-4-(2-oxopyridin-1(2H)-yl)phenyl)-1H-1,2,3-triazol-4-yl)methyl)-5-chlorothiophene-2-carboxamide |
| 70 | 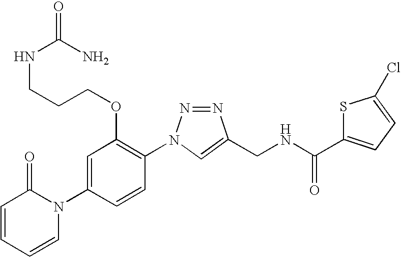 | 1-(3-(2-(4-((2-Chlorothiophene-5-carboxamido)methyl)-1H-1,2,3-triazol-1-yl)-5-(2-oxopyridin-1(2H)-yl)phenoxy)propyl)urea |
| 71 | 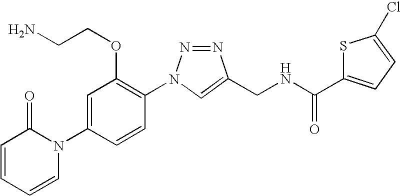 | N-((1-(2-(2-Aminoethoxy)-4-(2-oxopyridin-1(2H)-yl)phenyl)-1H-1,2,3-triazol-4-yl)methyl)-5-chlorothiophene-2-carboxamide |
| 72 | 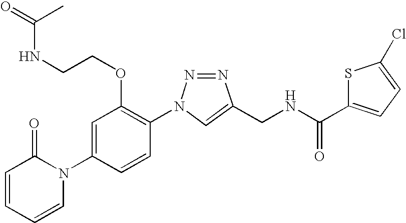 | N-((1-(2-(2-Acetamidoethoxy)-4-(2-oxopyridin-1(2H)-yl)phenyl)-1H-1,2,3-triazol-4-yl)methyl)-5-chlorothiophene-2-carboxamide |
| 73 | 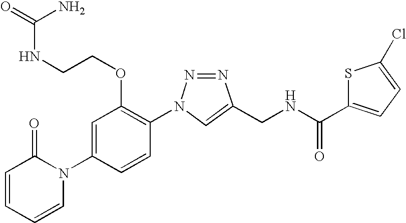 | 1-(2-(2-(4-((2-Chlorothiophene-5-carboxamido)methyl)-1H-1,2,3-triazol-1-yl)-5-(2-oxopyridin-1(2H)-yl)phenoxy)ethyl)urea |
| 74 | 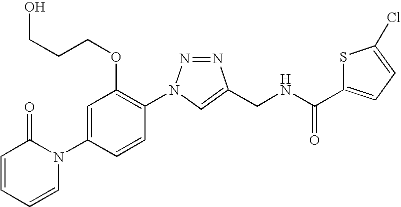 | 5-Chloro-N-((1-(2-(3-hydroxypropoxy)-4-(2-oxopyridin-1(2H)-yl)phenyl)-1H-1,2,3-triazol-4-yl)methyl)thiophene-2-carboxamide |
| 75 | 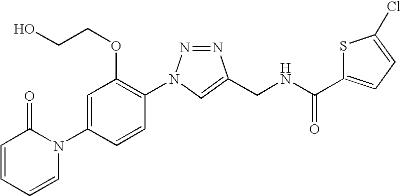 | 5-Chloro-N-((1-(2-(2-hydroxyethoxy)-4-(2-oxopyridin-1(2H)-yl)phenyl)-1H-1,2,3-triazol-4-yl)methyl)thiophene-2-carboxamide |
| 76 | 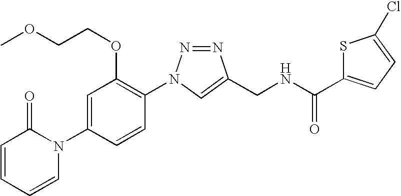 | 5-Chloro-N-((1-(2-(2-methoxyethoxy)-4-(2-oxopyridin-1(2H)-yl)phenyl)-1H-1,2,3-triazol-4-yl)methyl)thiophene-2-carboxamide |
| 77 | 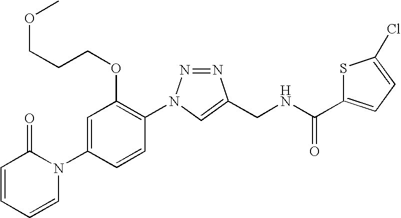 | 5-Chloro-N-((1-(2-(3-methoxypropoxy)-4-(2-oxopyridin-1(2H)-yl)phenyl)-1H-1,2,3-triazol-4-yl)methyl)thiophene-2-carboxamide |
| 78 | 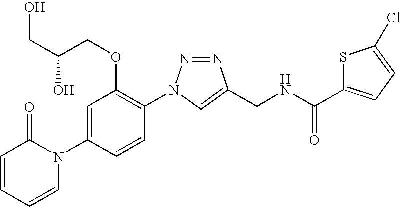 | 5-Chloro-N-((1-(2-((R)-2,3-dihydroxypropoxy)-4-(2-oxopyridin-1(2H)-yl)phenyl)-1H-1,2,3-triazol-4-yl)methyl)thiophene-2-carboxamide |
| 79 | 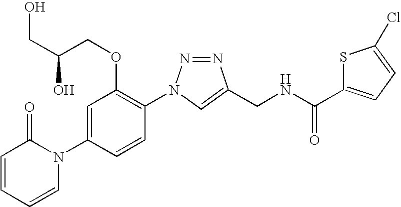 | 5-Chloro-N-((1-(2-((S)-2,3-dihydroxypropoxy)-4-(2-oxopyridin-1(2H)-yl)phenyl)-1H-1,2,3-triazol-4-yl)methyl)thiophene-2-carboxamide |
| 80 | 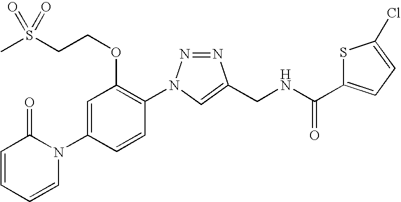 | 5-Chloro-N-((1-(2-(2-(methylsulfonyl)ethoxy)-4-(2-oxopyridin-1(2H)-yl)phenyl)-1H-1,2,3-triazol-4-yl)methyl)thiophene-2-carboxamide |
| 81 | 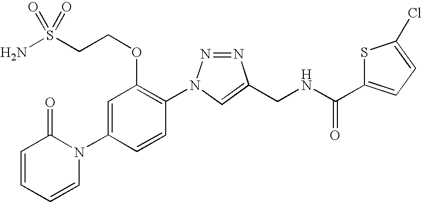 | 5-Chloro-N-((1-(2-(2-(aminosulfonyl)ethoxy)-4-(2-oxopyridin-1(2H)-yl)phenyl)-1H-1,2,3-triazol-4-yl)methyl)thiophene-2-carboxamide |
| 82 | 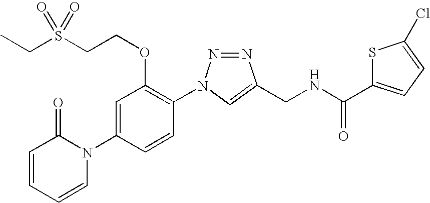 | 5-Chloro-N-((1-(2-(2-(ethylsulfonyl)ethoxy)-4-(2-oxopyridin-1(2H)-yl)phenyl)-1H-1,2,3-triazol-4-yl)methyl)thiophene-2-carboxamide |
| 83 | 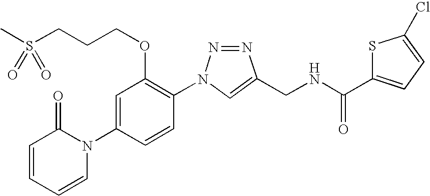 | 5-Chloro-N-((1-(2-(3-(methylsulfonyl)propoxy)-4-(2-oxopyridin-1(2H)-yl)phenyl)-1H-1,2,3-triazol-4-yl)methyl)thiophene-2-carboxamide |
| 84 | 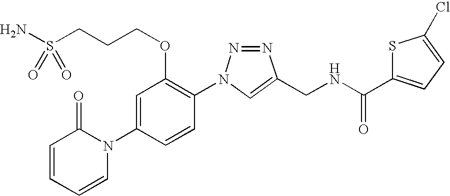 | 5-Chloro-N-((1-(2-(3-(aminosulfonyl)propoxy)-4-(2-oxopyridin-1(2H)-yl)phenyl)-1H-1,2,3-triazol-4-yl)methyl)thiophene-2-carboxamide |
| 85 | 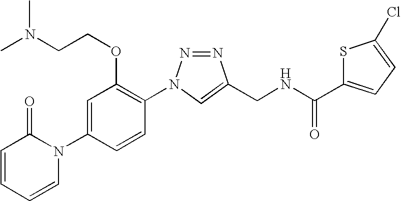 | 5-Chloro-N-((1-(2-(2-(dimethylamino)ethoxy)-4-(2-oxopyridin-1(2H)-yl)phenyl)-1H-1,2,3-triazol-4-yl)methyl)thiophene-2-carboxamide |
| 86 | 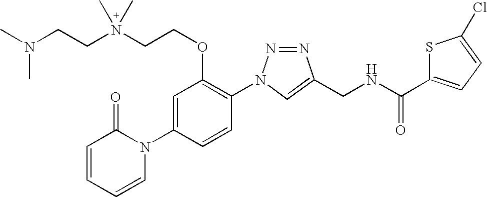 | 5-Chloro-N-((1-(2-(2-(dimethyl(dimethylamino)amino)ethoxy)-4-(2-oxopyridin-1(2H)-yl)phenyl)-1H-1,2,3-triazol-4-yl)methyl)thiophene-2-carboxamide |
| 87 | 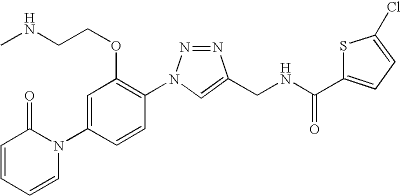 | 5-Chloro-N-((1-(2-(2-(methylamino)ethoxy)-4-(2-oxopyridin-1(2H)-yl)phenyl)-1H-1,2,3-triazol-4-yl)methyl)thiophene-2-carboxamide |
| 88 | 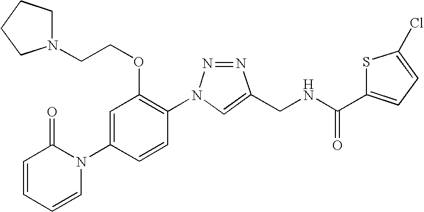 | 5-Chloro-N-((1-(4-(2-oxopyridin-1(2H)-yl)-2-(2-(pyrrolidin-1-yl)ethoxy)phenyl)-1H-1,2,3-triazol-4-yl)methyl)thiophene-2-carboxamide |
| 89 | 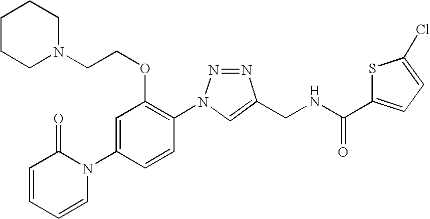 | 5-Chloro-N-((1-(4-(2-oxopyridin-1(2H)-yl)-2-(2-(piperidin-1-yl)ethoxy)phenyl)-1H-1,2,3-triazol-4-yl)methyl)thiophene-2-carboxamide |
| 90 | 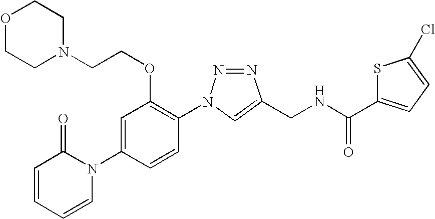 | 5-Chloro-N-((1-(2-(2-morpholinoethoxy)-4-(2-oxopyridin-1(2H)-yl)phenyl)-1H-1,2,3-triazol-4-yl)methyl)thiophene-2-carboxamide |
| 91 | 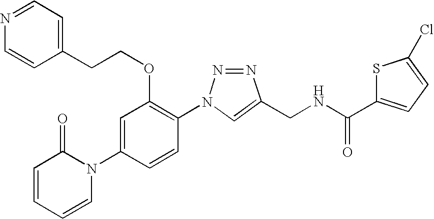 | 5-Chloro-N-((1-(4-(2-oxopyridin-1(2H)-yl)-2-(2-(pyridin-4-yl)ethoxy)phenyl)-1H-1,2,3-triazol-4-yl)methyl)thiophene-2-carboxamide |
| 92 | 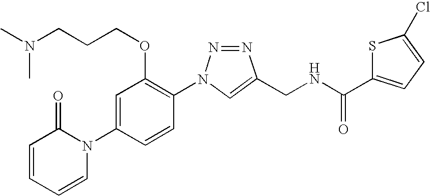 | 5-Chloro-N-((1-(2-(3-(dimethylamino)propoxy)-4-(2-oxopyridin-1(2H)-yl)phenyl)-1H-1,2,3-triazol-4-yl)methyl)thiophene-2-carboxamide |
| 93 | 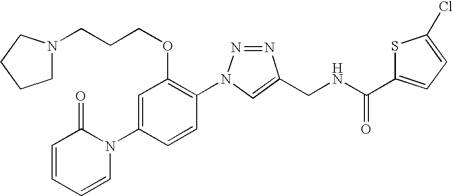 | 5-Chloro-N-((1-(4-(2-oxopyridin-1(2H)-yl)-2-(3-(pyrrolidin-1-yl)propoxy)phenyl)-1H-1,2,3-triazol-4-yl)methyl)thiophene-2-carboxamide |
| 94 | 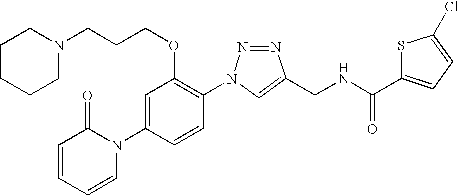 | 5-Chloro-N-((1-(4-(2-oxopyridin-1(2H)-yl)-2-(3-(piperidin-1-yl)propoxy)phenyl)-1H-1,2,3-triazol-4-yl)methyl)thiophene-2-carboxamide |
| 95 | 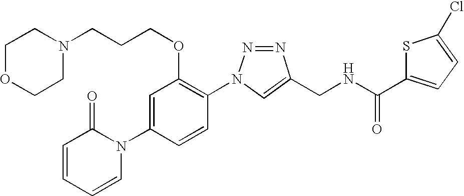 | 5-Chloro-N-((1-(2-(3-morpholinopropoxy)-4-(2-oxopyridin-1(2H)-yl)phenyl)-1H-1,2,3-triazol-4-yl)methyl)thiophene-2-carboxamide |
| 96 | 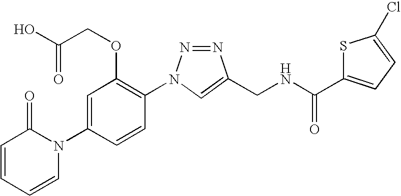 | 2-(2-(4-((2-Chlorothiophene-5-carboxamido)methyl)-1H-1,2,3-triazol-1-yl)-5-(2-oxopyridin-1(2H)-yl)phenoxy)acetic acid |
| 97 | 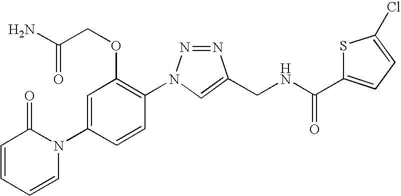 | N-((1-(2-(2-Amino-2-oxoethoxy)-4-(2-oxopyridin-1(2H)-yl)phenyl)-1H-1,2,3-triazol-4-yl)methyl)-5-chlorothiophene-2-carboxamide |
| 98 | 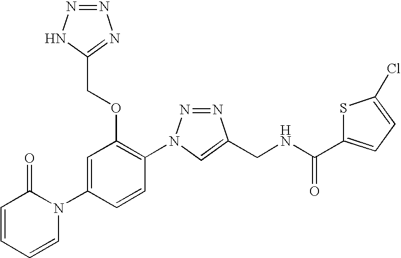 | N-((1-(2-((1H-Tetrazol-5-yl)methoxy)-4-(2-oxopyridin-1(2H)-yl)phenyl)-1H-1,2,3-triazol-4-yl)methyl)-5-chlorothiophene-2-carboxamide |
| 99 | 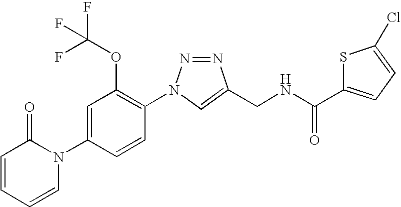 | 5-Chloro-N-((1-(4-(2-oxopyridin-1(2H)-yl)-2-(trifluoromethoxy)phenyl)-1H-1,2,3-triazol-4-yl)methyl)thiophene-2-carboxamide |
| 100 | 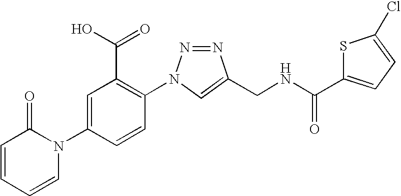 | 2-(4-((2-Chlorothiophene-5-carboxamido)methyl)-1H-1,2,3-triazol-1-yl)-5-(2-oxopyridin-1(2H)-yl)benzoic acid |
| 101 | 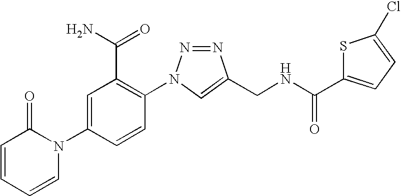 | N-((1-(2-Carbamoyl-4-(2-oxopyridin-1(2H)-yl)phenyl)-1H-1,2,3-triazol-4-yl)methyl)-5-chlorothiophene-2-carboxamide |
| 102 | 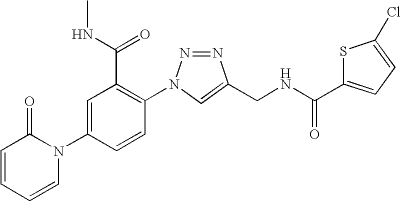 | 5-Chloro-N-((1-(2-(methylcarbamoyl)-4-(2-oxopyridin-1(2H)-yl)phenyl)-1H-1,2,3-triazol-4-yl)methyl)thiophene-2-carboxamide |
| 103 | 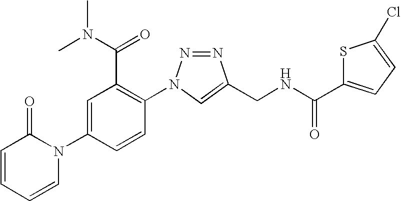 | 5-Chloro-N-((1-(2-(dimethylcarbamoyl)-4-(2-oxopyridin-1(2H)-yl)phenyl)-1H-1,2,3-triazol-4-yl)methyl)thiophene-2-carboxamide |
| 104 | 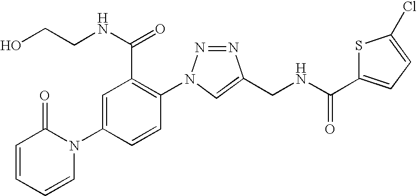 | N-((1-(2-((2-Hydroxyethyl)carbamoyl)-4-(2-oxopyridin-1(2H)-yl)phenyl)-1H-1,2,3-triazol-4-yl)methyl)-5-chlorothiophene-2-carboxamide |
| 105 | 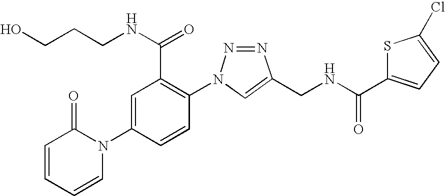 | N-((1-(2-((3-Hydroxypropyl)carbamoyl)-4-(2-oxopyridin-1(2H)-yl)phenyl)-1H-1,2,3-triazol-4-yl)methyl)-5-chlorothiophene-2-carboxamide |
| 106 | 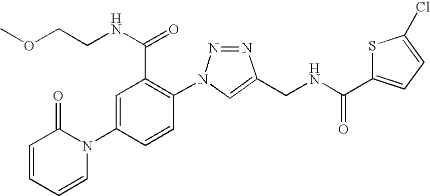 | N-((1-(2-((2-Methoxyethyl)carbamoyl)-4-(2-oxopyridin-1(2H)-yl)phenyl)-1H-1,2,3-triazol-4-yl)methyl)-5-chlorothiophene-2-carboxamide |
| 107 | 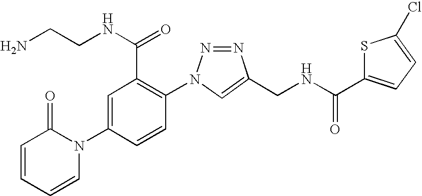 | N-((1-(2-((2-Aminoethyl)carbamoyl)-4-(2-oxopyridin-1(2H)-yl)phenyl)-1H-1,2,3-triazol-4-yl)methyl)-5-chlorothiophene-2-carboxamide |
| 108 | 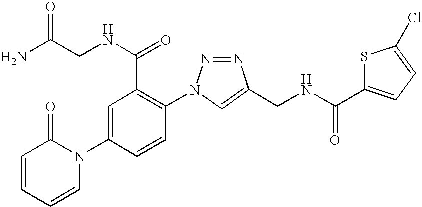 | N-((1-(2-((2-Amino-2-oxoethyl)carbamoyl)-4-(2-oxopyridin-1(2H)-yl)phenyl)-1H-1,2,3-triazol-4-yl)methyl)-5-chlorothiophene-2-carboxamide |
| 109 | 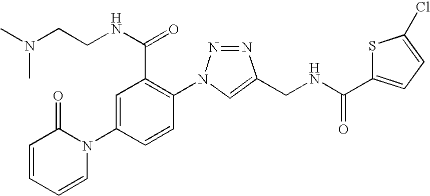 | N-((1-(2-((2-(Dimethylamino)ethyl)carbamoyl)-4-(2-oxopyridin-1(2H)-yl)phenyl)-1H-1,2,3-triazol-4-yl)methyl)-5-chlorothiophene-2-carboxamide |
| 110 | 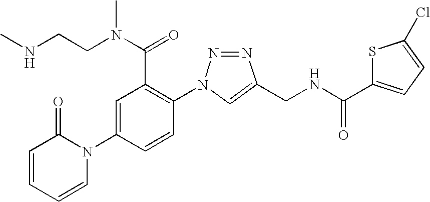 | 5-Chloro-N-((1-(2-(methyl(2-(methylamino)ethyl)carbamoyl)-4-(2-oxopyridin-1(2H)-yl)phenyl)-1H-1,2,3-triazol-4-yl)methyl)thiophene-2-carboxamide |
| 111 | 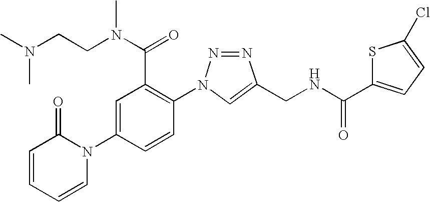 | 5-Chloro-N-((1-(2-((2-(dimethylamino)ethyl)(methyl)carbamoyl)-4-(2-oxopyridin-1(2H)-yl)phenyl)-1H-1,2,3-triazol-4-yl)methyl)thiophene-2-carboxamide |
| 112 | 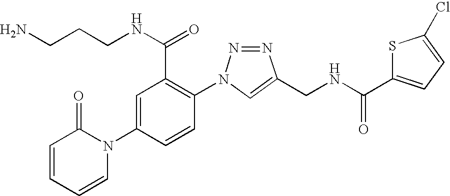 | N-((1-(2-(3-aminopropylcarbamoyl)-4-(2-oxopyridin-1(2H)-yl)phenyl)-1H-1,2,3-triazol-4-yl)methyl)-5-chlorothiophene-2-carboxamide |
| 113 | 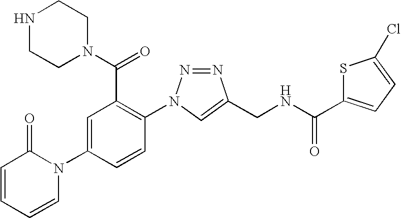 | 5-Chloro-N-((1-(4-(2-oxopyridin-1(2H)-yl)-2-(piperazine-1-carbonyl)phenyl)-1H-1,2,3-triazol-4-yl)methyl)thiophene-2-carboxamide |
| 114 | 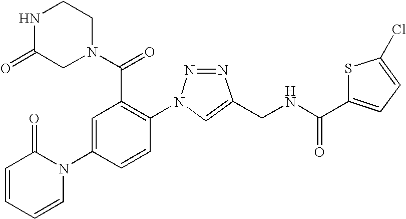 | 5-Chloro-N-((1-(2-(2-oxopiperazine-4-carbonyl)-4-(2-oxopyridin-1(2H)-yl)phenyl)-1H-1,2,3-triazol-4-yl)methyl)thiophene-2-carboxamide |
| 115 | 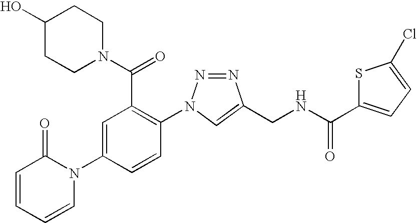 | 5-Chloro-N-((1-(2-(4-hydroxypiperidine-1-carbonyl)-4-(2-oxopyridin-1(2H)-yl)phenyl)-1H-1,2,3-triazol-4-yl)methyl)thiophene-2-carboxamide |
| 116 | 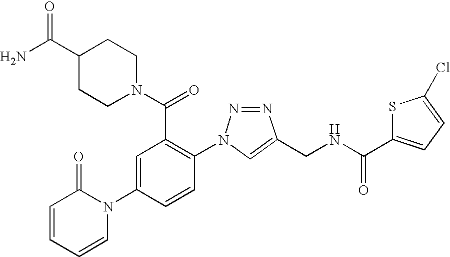 | 1-(2-(4-((5-Chlorothiophene-2-carboxamido)methyl)-1H-1,2,3-triazol-1-yl)-5-(2-oxopyridin-1(2H)-yl)benzoyl)piperidine-4-carboxamide |
| 117 | 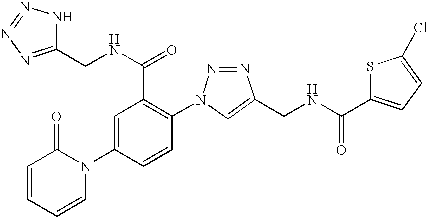 | N-((1-(2-(((1H-Tetrazol-5-yl)methyl)carbamoyl)-4-(2-oxopyridin-1(2H)-yl)phenyl)-1H-1,2,3-triazol-4-yl)methyl)-5-chlorothiophene-2-carboxamide |
| 118 | 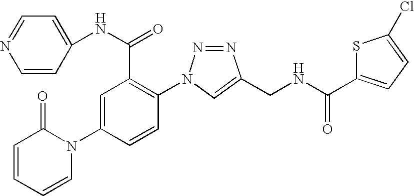 | 5-Chloro-N-((1-(4-(2-oxopyridin-1(2H)-yl)-2-(pyridin-4-ylcarbamoyl)phenyl)-1H-1,2,3-triazol-4-yl)methyl)thiophene-2-carboxamide |
| 119 | 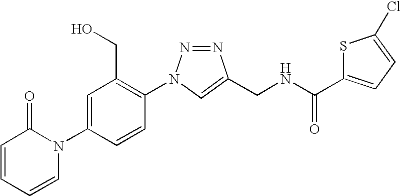 | 5-Chloro-N-((1-(2-(hydroxymethyl)-4-(2-oxopyridin-1(2H)-yl)phenyl)-1H-1,2,3-triazol-4-yl)methyl)thiophene-2-carboxamide |
| 120 | 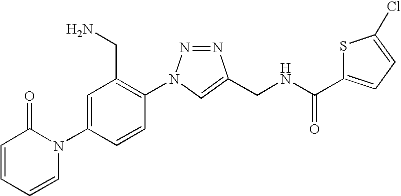 | N-((1-(2-(Aminomethyl)-4-(2-oxopyridin-1(2H)-yl)phenyl)-1H-1,2,3-triazol-4-yl)methyl)-5-chlorothiophene-2-carboxamide |
| 121 | 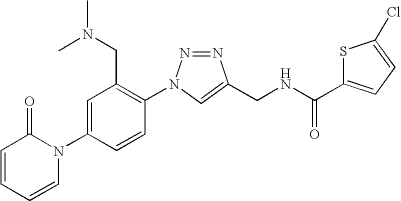 | 5-Chloro-N-((1-(2-((dimethylamino)methyl)-4-(2-oxopyridin-1(2H)-yl)phenyl)-1H-1,2,3-triazol-4-yl)methyl)thiophene-2-carboxamide |
| 122 | 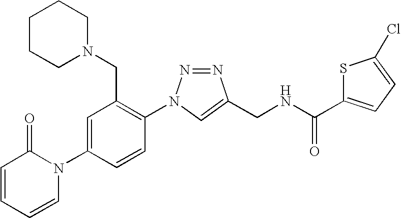 | 5-Chloro-N-((1-(4-(2-oxopyridin-1(2H)-yl)-2-(piperidin-1-ylmethyl)phenyl)-1H-1,2,3-triazol-4-yl)methyl)thiophene-2-carboxamide |
| 123 | 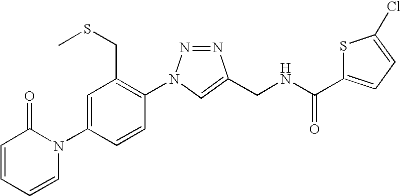 | 5-Chloro-N-((1-(2-(methylthiomethyl)-4-(2-oxopyridin-1(2H)-yl)phenyl)-1H-1,2,3-triazol-4-yl)methyl)thiophene-2-carboxamide |
| 124 | 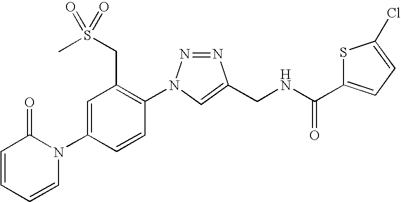 | 5-Chloro-N-((1-(2-(methylsulfonylmethyl)-4-(2-oxopyridin-1(2H)-yl)phenyl)-1H-1,2,3-triazol-4-yl)methyl)thiophene-2-carboxamide |
| 125 | 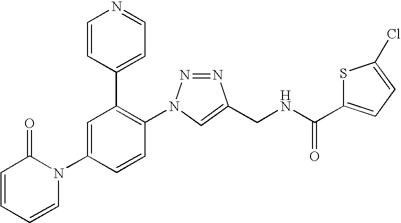 | 5-Chloro-N-((1-(4-(2-oxopyridin-1(2H)-yl)-2-(pyridin-4-yl)phenyl)-1H-1,2,3-triazol-4-yl)methyl)thiophene-2-carboxamide |
| 126 | 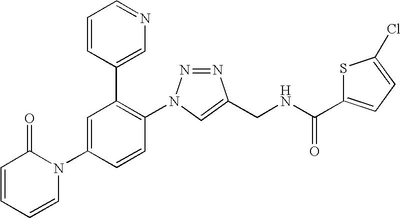 | 5-Chloro-N-((1-(4-(2-oxopyridin-1(2H)-yl)-2-(pyridin-3-yl)phenyl)-1H-1,2,3-triazol-4-yl)methyl)thiophene-2-carboxamide |
| 127 | 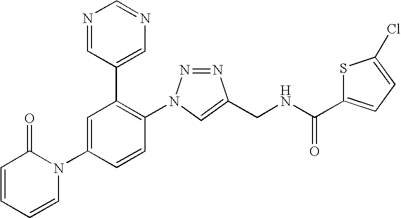 | 5-Chloro-N-((1-(4-(2-oxopyridin-1(2H)-yl)-2-(pyrimidin-5-yl)phenyl)-1H-1,2,3-triazol-4-yl)methyl)thiophene-2-carboxamide |
| 128 | 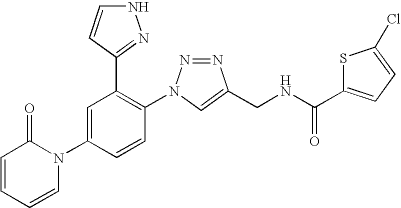 | 5-Chloro-N-((1-(4-(2-oxopyridin-1(2H)-yl)-2-(1H-pyrazol-3-yl)phenyl)-1H-1,2,3-triazol-4-yl)methyl)thiophene-2-carboxamide |
| 129 | 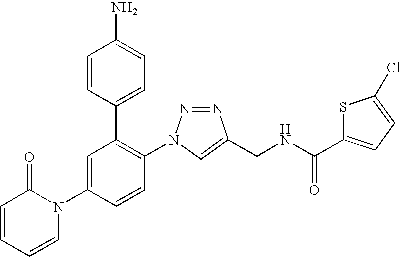 | 5-Chloro-N-((1-(4-(2-oxopyridin-1(2H)-yl)-2-((4-aminophenyl)phenyl)-1H-1,2,3-triazol-4-yl)methyl)thiophene-2-carboxamide |
| 130 | 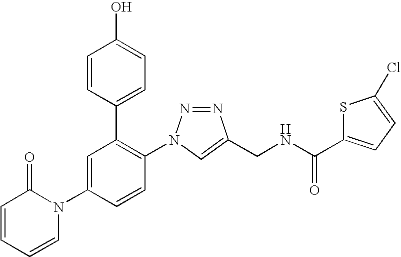 | 5-Chloro-N-((1-(4-(2-oxopyridin-1(2H)-yl)-2-((4-hydroxyphenyl)phenyl)-1H-1,2,3-triazol-4-yl)methyl)thiophene-2-carboxamide |
| 131 | 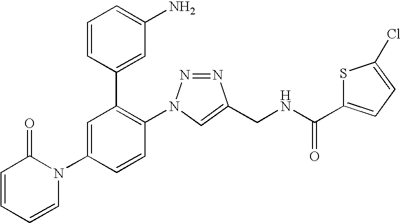 | 5-Chloro-N-((1-(4-(2-oxopyridin-1(2H)-yl)-2-((3-aminophenyl)phenyl)-1H-1,2,3-triazol-4-yl)methyl)thiophene-2-carboxamide |
| 132 | 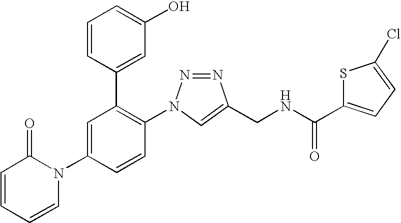 | 5-Chloro-N-((1-(4-(2-oxopyridin-1(2H)-yl)-2-((3-hydroxyphenyl)phenyl)-1H-1,2,3-triazol-4-yl)methyl)thiophene-2-carboxamide |
| 133 | 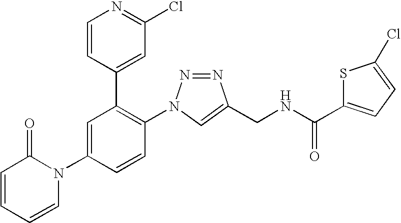 | 5-Chloro-N-((1-(2-(2-chloropyridin-4-yl)-4-(2-oxopyridin-1(2H)-yl)phenyl)-1H-1,2,3-triazol-4-yl)methyl)thiophene-2-carboxamide |
| 134 | 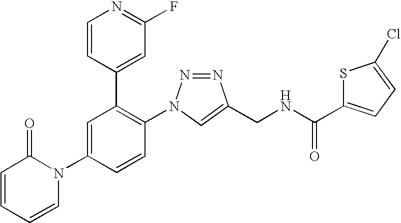 | 5-Chloro-N-((1-(2-(2-fluoropyridin-4-yl)-4-(2-oxopyridin-1(2H)-yl)phenyl)-1H-1,2,3-triazol-4-yl)methyl)thiophene-2-carboxamide |
| 135 | 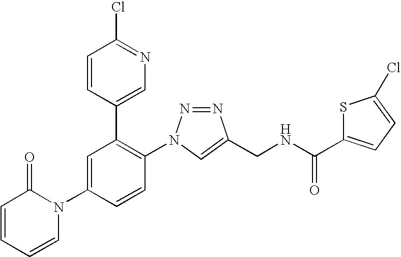 | 5-Chloro-N-((1-(2-(6-chloropyridin-3-yl)-4-(2-oxopyridin-1(2H)-yl)phenyl)-1H-1,2,3-triazol-4-yl)methyl)thiophene-2-carboxamide |
| 136 | 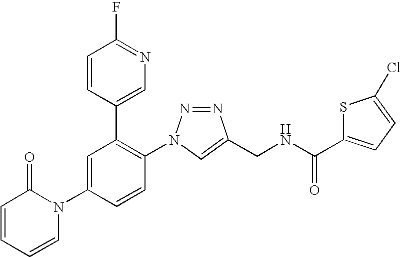 | 5-Chloro-N-((1-(2-(6-fluoropyridin-3-yl)-4-(2-oxopyridin-1(2H)-yl)phenyl)-1H-1,2,3-triazol-4-yl)methyl)thiophene-2-carboxamide |
| 137 | 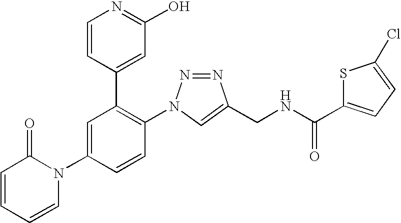 | 5-Chloro-N-((1-(2-(2-hydroxypyridin-4-yl)-4-(2-oxopyridin-1(2H)-yl)phenyl)-1H-1,2,3-triazol-4-yl)methyl)thiophene-2-carboxamide |
| 138 | 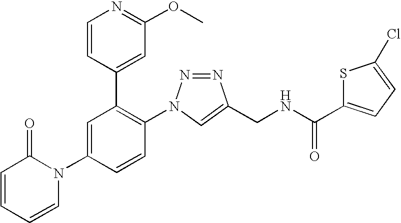 | 5-Chloro-N-((1-(2-(2-methoxypyridin-4-yl)-4-(2-oxopyridin-1(2H)-yl)phenyl)-1H-1,2,3-triazol-4-yl)methyl)thiophene-2-carboxamide |
| 139 | 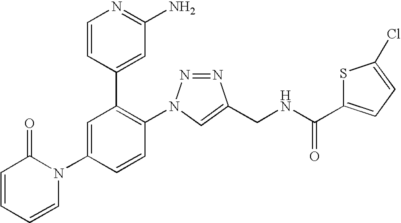 | N-((1-(2-(2-Aminopyridin-4-yl)-4-(2-oxopyridin-1(2H)-yl)phenyl)-1H-1,2,3-triazol-4-yl)methyl)-5-chlorothiophene-2-carboxamide |
| 140 | 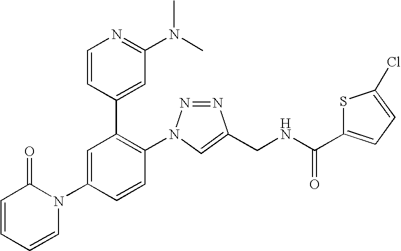 | 5-Chloro-N-((1-(2-(2-(dimethylamino)pyridin-4-yl)-4-(2-oxopyridin-1(2H)-yl)phenyl)-1H-1,2,3-triazol-4-yl)methyl)thiophene-2-carboxamide |
| 141 | 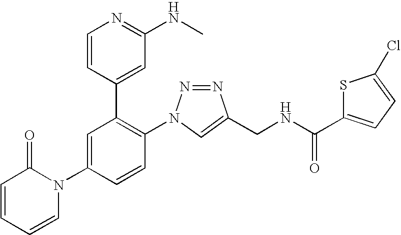 | 5-Chloro-N-((1-(2-(2-(methylamino)pyridin-4-yl)-4-(2-oxopyridin-1(2H)-yl)phenyl)-1H-1,2,3-triazol-4-yl)methyl)thiophene-2-carboxamide |
| 142 | 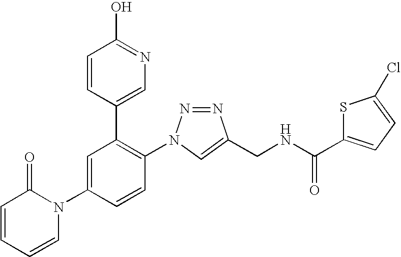 | 5-Chloro-N-((1-(2-(6-hydroxypyridin-3-yl)-4-(2-oxopyridrn-1(2H)-yl)phenyl)-1H-1,2,3-triazol-4-yl)methyl)thiophene-2-carboxamide |
| 143 | 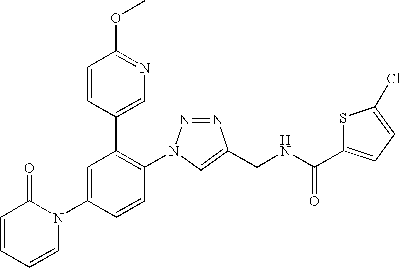 | 5-Chloro-N-((1-(2-(6-methoxypyridin-3-yl)-4-(2-oxopyridin-1(2H)-yl)phenyl)-1H-1,2,3-triazol-4-yl)methyl)thiophene-2-carboxamide |
| 144 | 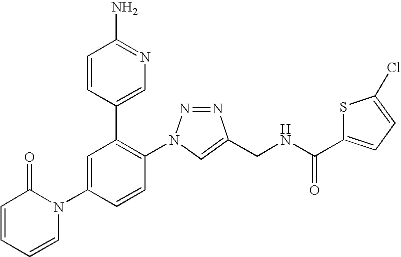 | N-((1-(2-(6-Aminopyridin-3-yl)-4-(2-oxopyridin-1(2H)-yl)phenyl)-1H-1,2,3-triazol-4-yl)methyl)-5-chlorothiophene-2-carboxamide |
| 145 | 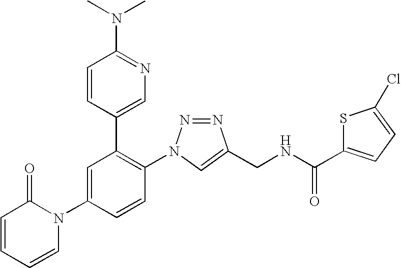 | 5-Chloro-N-((1-(2-(6-(dimethylamino)pyridin-3-yl)-4-(2-oxopyridin-1(2H)-yl)phenyl)-1H-1,2,3-triazol-4-yl)methyl)thiophene-2-carboxamide |
| 146 | 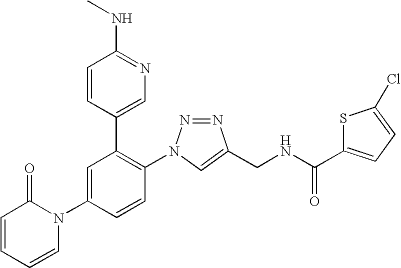 | 5-Chloro-N-((1-(2-(6-(methylamino)pyridin-3-yl)-4-(2-oxopyridin-1(2H)-yl)phenyl)-1H-1,2,3-triazol-4-yl)methyl)thiophene-2-carboxamide |
| 147 | 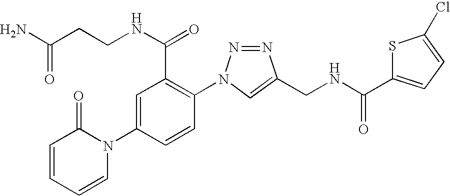 | N-((1-(2-(3-Amino-3-oxopropylcarbamoyl)-4-(2-oxopyridin-1(2H)-yl)phenyl)-1H-1,2,3-triazol-4-yl)methyl)-5-chlorothiophene-2-carboxamide |
| 148 | 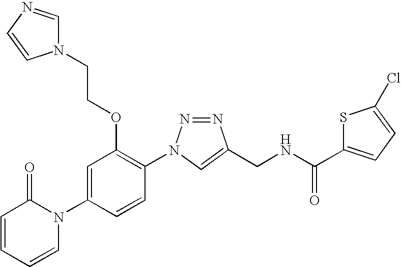 | N-((1-(2-(2-(1H-Imidazol-1-yl)ethoxy)-4-(2-oxopyridin-1(2H)-yl)phenyl)-1H-1,2,3-triazol-4-yl)methyl)-5-chlorothiophene-2-carboxamide |
| 149 | 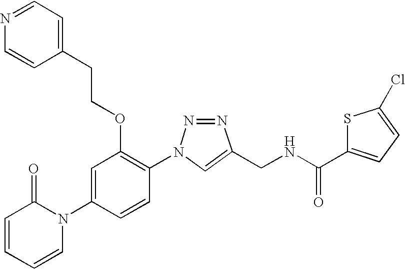 | 5-Chloro-N-((1-(4-(2-oxopyridin-1(2H)-yl)-2-(2-(pyridin-4-yl)ethoxy)phenyl)-1H-1,2,3-triazol-4-yl)methyl)thiophene-2-carboxamide |
| 150 | 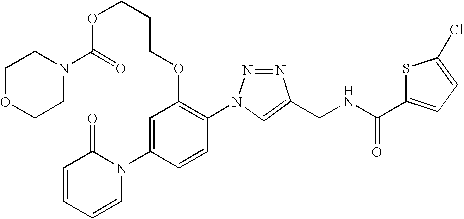 | 3-(2-(4-((5-Chlorothiophene-2-carboxamido)methyl)-1H-1,2,3-triazol-1-yl)-5-(2-oxopyridin-1(2H)-yl)phenoxy)propyl morpholine-4-carboxylate |
| 151 | 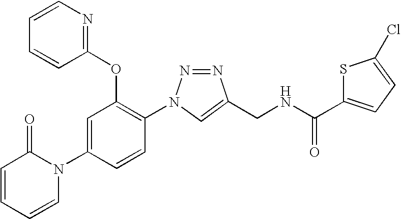 | 5-Chloro-N-((1-(4-(2-oxopyridin-1(2H)-yl)-2-(pyridin-2-yloxy)phenyl)-1H-1,2,3-triazol-4-yl)methyl)thiophene-2-carboxamid |
| 152 | 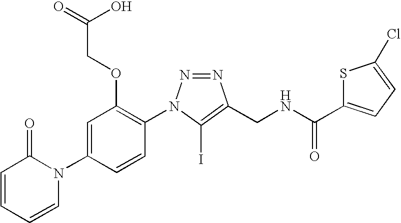 | 2-(2-(4-((5-Chlorothiophene-2-carboxamido)methyl)-5-iodo-1H-1,2,3-triazol-1-yl)-5-(2-oxopyridin-1(2H)-yl)phenoxy)acetic acid |
| 153 | 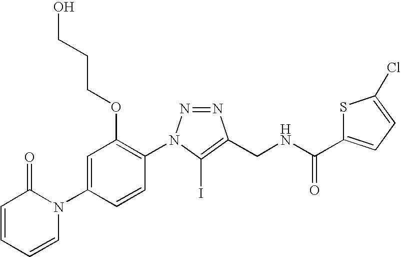 | 5-Chloro-N-((1-(2-(3-hydroxypropoxy)-4-(2-oxopyridin-1(2H)-yl)phenyl)-5-iodo-1H-1,2,3-triazol-4-yl)methyl)thiophene-2-carboxamide |
| 154 | 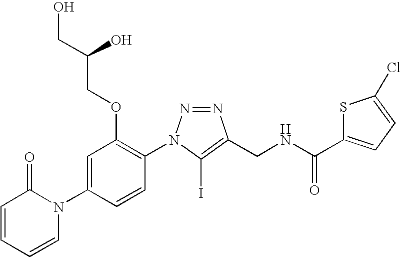 | 5-Chloro-N-((1-(2-((R)-2,3-dihydroxypropoxy)-4-(2-oxopyridin-1(2H)-yl)phenyl)-5-iodo-1H-1,2,3-triazol-4-yl)methyl)thiophene-2-carboxamide |
| 155 | 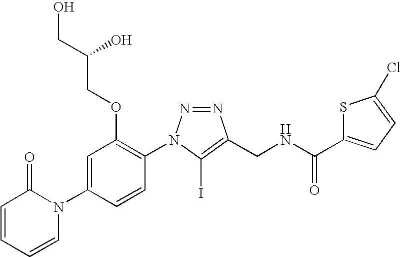 | 5-Chloro-N-((1-(2-((S)-2,3-dihydroxypropoxy)-4-(2-oxopyridin-1(2H)-yl)phenyl)-5-iodo-1H-1,2,3-triazol-4-yl)methyl)thiophene-2-carboxamide |
| 156 | 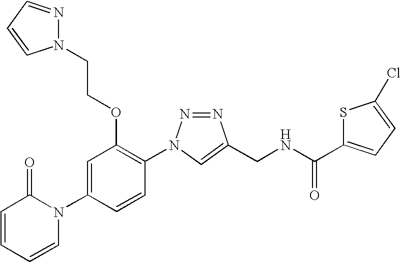 | N-((1-(2-(2-(1H-Pyrazol-1-yl)ethoxy)-4-(2-oxopyridin-1(2H)-yl)phenyl)-1H-1,2,3-triazol-4-yl)methyl)-5-chlorothiophene-2-carboxamide |
| 157 | 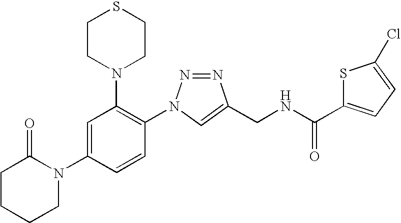 | 5-Chloro-N-((1-(4-(2-oxopiperidin-1-yl)-2-thiomorpholinophenyl)-1H-1,2,3-triazol-4-yl)methyl)thiophene-2-carboxamide |
| 158 | 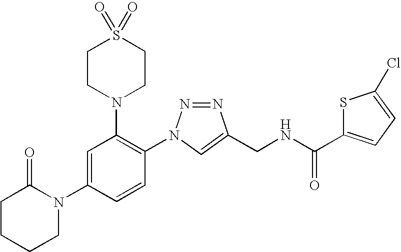 | 5-Chloro-N-((1-(4-(2-oxopiperidin-1-yl)-2-((1,1-dioxo)thiomorpholino)phenyl)-1H-1,2,3-triazol-4-yl)methyl)thiophene-2-carboxamide |
| 159 | 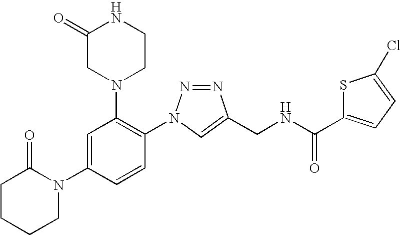 | 5-Chloro-N-((1-(2-(3-oxopiperazin-1-yl)-4-(2-oxopiperidin-1-yl)phenyl)-1H-1,2,3-triazol-4-yl)methyl)thiophene-2-carboxamide |
| 160 | 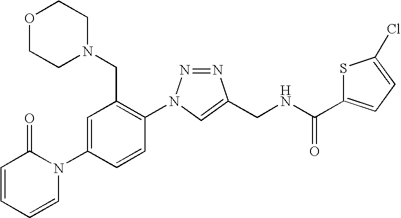 | 5-Chloro-N-((1-(2-(morpholinomethyl)-4-(2-oxopyridin-1(2H)-yl)phenyl)-1H-1,2,3-triazol-4-yl)methyl)thiophene-2-carboxamide |
| 161 | 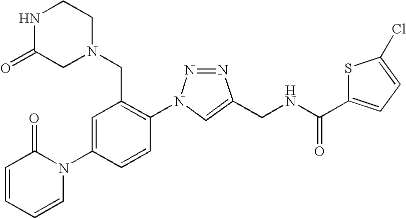 | 5-Chloro-N-((1-(2-((3-oxopiperazin-1-yl)methyl)-4-(2-oxopyridin-1(2H)-yl)phenyl)-1H-1,2,3-triazol-4-yl)methyl)thiophene-2-carboxamide |
| 162 | 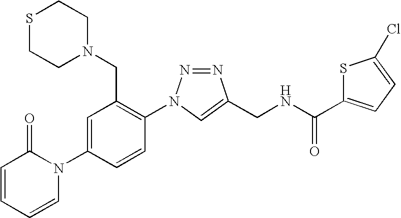 | 5-Chloro-N-((1-(4-(2-oxopyridin-1(2H)-yl)-2-(thiomorpholinomethyl)phenyl)-1H-1,2,3-triazol-4-yl)methyl)thiophene-2-carboxamide |
| 163 | 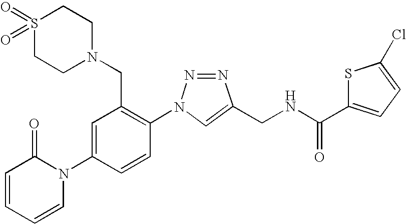 | 5-Chloro-N-((1-(4-(2-oxopyridin-1(2H)-yl)-2-(1,1,-dioxothiomorpholinomethyl)phenyl)-1H-1,2,3-triazol-4-yl)methyl)thiophene-2-carboxamide |
| 164 | 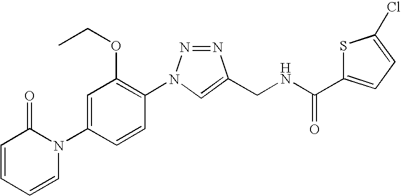 | 5-Chloro-N-((1-(2-ethoxy-4-(2-oxopyridin-1(2H)-yl)phenyl)-1H-1,2,3-triazol-4-yl)methyl)thiophene-2-carboxamide |
| 165 | 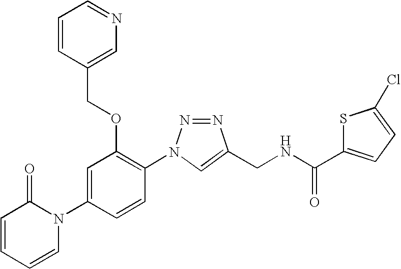 | 5-Chloro-N-((1-(4-(2-oxopyridin-1(2H)-yl)-2-(pyridin-3-ylmethoxy)phenyl)-1H-1,2,3-triazol-4-yl)methyl)thiophene-2-carboxamide |
| 166 | 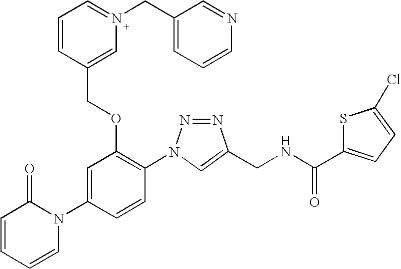 | 5-Chloro-N-((1-(4-(2-oxopyridin-1(2H)-yl)-2-(N-(pyridine-3-yl)pyridin-3-ylmethoxy)phenyl)-1H-1,2,3-triazol-4-yl)methyl)thiophene-2-carboxamide |
| 167 | 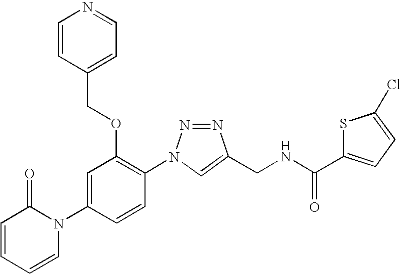 | 5-Chloro-N-((1-(4-(2-oxopyridin-1(2H)-yl)-2-(pyridin-4-ylmethoxy)phenyl)-1H-1,2,3-triazol-4-yl)methyl)thiophene-2-carboxamide |
| 168 | 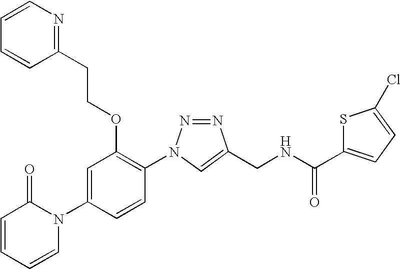 | 5-Chloro-N-((1-(4-(2-oxopyridin-1(2H)-yl)-2-(2-(pyridin-2-yl)ethoxy)phenyl)-1H-1,2,3-triazol-4-yl)methyl)thiophene-2-carboxamide |
| 169 | 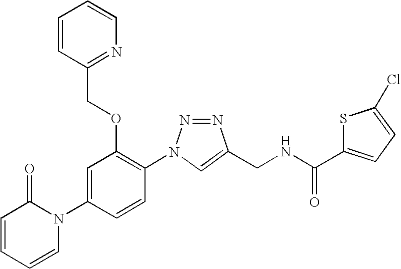 | 5-Chloro-N-((1-(4-(2-oxopyridin-1(2H)-yl)-2-(pyridin-2-ylmethoxy)phenyl)-1H-1,2,3-triazol-4-yl)methyl)thiophene-2-carboxamide |
| 170 | 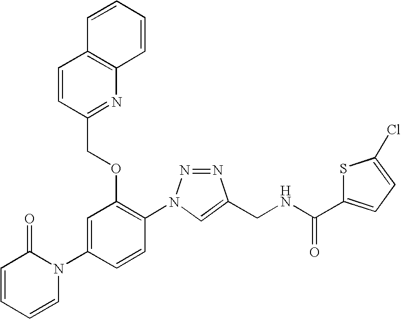 | 5-Chloro-N-((1-(4-(2-oxopyridin-1(2H)-yl)-2-(quinolin-2-ylmethoxy)phenyl)-1H-1,2,3-triazol-4-yl)methyl)thiophene-2-carboxamide |
| 171 | 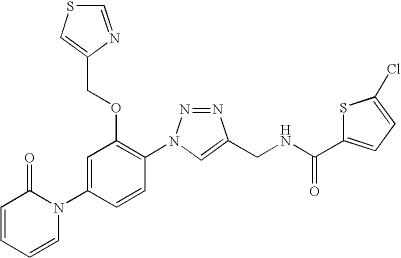 | 5-Chloro-N-((1-(4-(2-oxopyridin-1(2H)-yl)-2-(thiazol-4-ylmethoxy)phenyl)-1H-1,2,3-triazol-4-yl)methyl)thiophene-2-carboxamide |
| 172 | 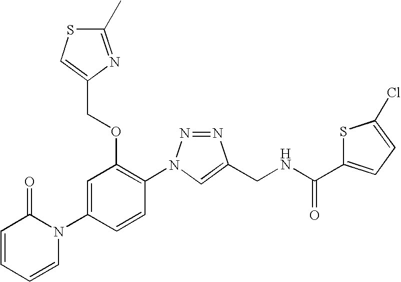 | 5-Chloro-N-((1-(2-((2-methylthiazol-4-yl)methoxy)-4-(2-oxopyridin-1(2H)-yl)phenyl)-1H-1,2,3-triazol-4-yl)methyl)thiophene-2-carboxamide |
| 173 | 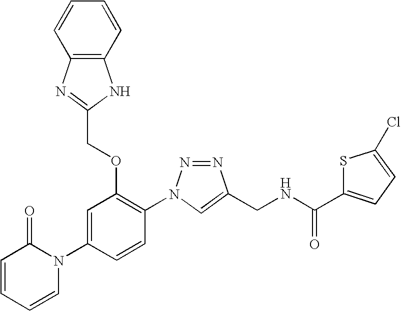 | N-((1-(2-((1H-Benzo[d]imidazol-2-yl)methoxy)-4-(2-oxopyridin-1(2H)-yl)phenyl)-1H-1,2,3-triazol-4-yl)methyl)-5-chlorothiophene-2-carboxamide |
| 174 | 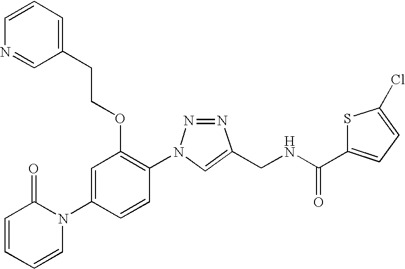 | 5-Chloro-N-((1-(4-(2-oxopyridin-1(2H)-yl)-2-(2-(pyridin-3-yl)ethoxy)phenyl)-1H-1,2,3-triazol-4-yl)methyl)thiophene-2-carboxamide |
| 175 | 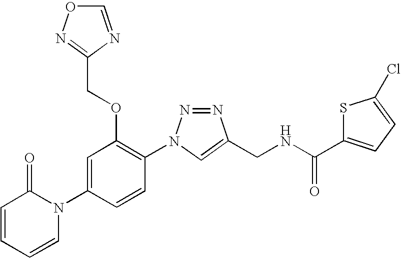 | N-((1-(2-((1,2,4-Oxadiazol-3-yl)methoxy)-4-(2-oxopyridin-1(2H)-yl)phenyl)-1H-1,2,3-triazol-4-yl)methyl)-5-chlorothiophene-2-carboxamide |
| 176 | 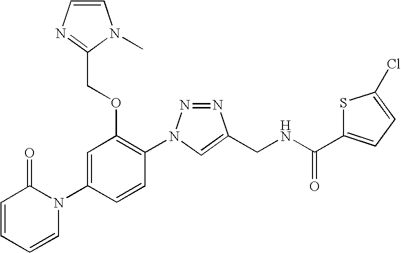 | 5-Chloro-N-((1-(2-((1-methyl-1H-imidazol-2-yl)methoxy)-4-(2-oxopyridin-1(2H)-yl)phenyl)-1H-1,2,3-triazol-4-yl)methyl)thiophene-2-carboxamide |
| 177 | 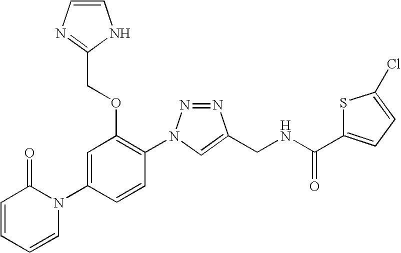 | N-((1-(2-((1H-Imidazol-2-yl)methoxy)-4-(2-oxopyridin-1(2H)-yl)phenyl)-1H-1,2,3-triazol-4-yl)methyl)-5-chlorothiophene-2-carboxamide |
| 178 | 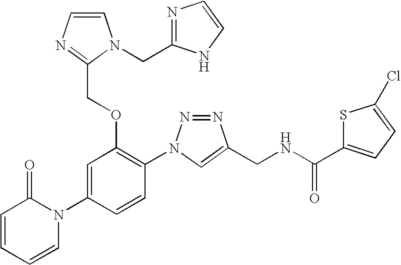 | N-((1-(2-((1-((1H-imidazol-2-yl)methyl)-1H-imidazol-2-yl)methoxy)-4-(2-oxopyridin-1(2H)-yl)phenyl)-1H-1,2,3-triazol-4-yl)methyl)-5-chlorothiophene-2-carboxamide |
| 179 | 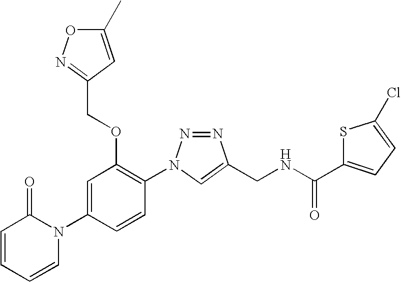 | 5-Chloro-N-((1-(2-((5-methylisoxazol-3-yl)methoxy)-4-(2-oxopyridin-1(2H)-yl)phenyl)-1H-1,2,3-triazol-4-yl)methyl)thiophene-2-carboxamide |
| 180 | 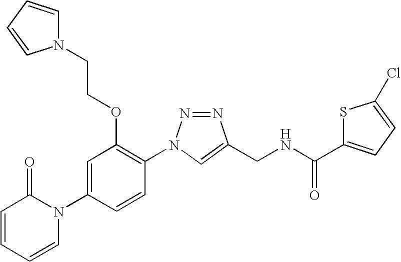 | N-((1-(2-(2-(1H-Pyrrol-1-yl)ethoxy)-4-(2-oxopyridin-1(2H)-yl)phenyl)-1H-1,2,3-triazol-4-yl)methyl)-5-chlorothiophene-2-carboxamide |

All the preferred, more preferred, and most preferred compounds listed above are selective inhibitors of Factor Xa.

### Compositions

The present invention further provides compositions comprising one or more compounds of Formula (I), (II), (Ia), or (Ib) or a pharmaceutically acceptable salt, ester, or prodrug thereof and a pharmaceutically acceptable carrier. It will be appreciated that the compounds of Formula (I), (II), (Ia), or (Ib) in this invention may be derivatized at functional groups to provide prodrug derivatives which are capable of conversion back to the parent compounds in vivo. Examples of such prodrugs include the physiologically acceptable and metabolically labile ester derivatives, such as methoxymethyl esters, methylthiomethyl esters, or pivaloyloxymethyl esters derived from a hydroxyl group of the compound or a carbamoyl moiety derived from an amino group of the compound. Additionally, any physiologically acceptable equivalents of the compounds of Formula (I), (II), (Ia), or (Ib) similar to metabolically labile esters or carbamates, which are capable of producing the parent compounds of Formula (I), (II), (Ia), or (Ib) in vivo, are within the scope of this invention.

If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Furthermore, the basic nitrogen-containing groups may be quaternized with agents like lower alkyl halides, such as methyl, ethyl, propyl and butyl chlorides, bromides and iodides; dialkyl sulfates, such as dimethyl, diethyl, dibutyl and diamyl sulfates, long chain halides, such as decyl, lauryl, myristyl and stearyl chlorides, bromides and iodides; aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, central nervous system, etc.), increase oral availability, increase solubility to allow administration by injection, alter metabolism and alter rate of excretion.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

Pharmaceutically acceptable carriers that may be used in these compositions include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to a preferred embodiment, the compositions of this invention are formulated for pharmaceutical administration to a mammal, preferably a human being. Such pharmaceutical compositions of the invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. The formulations of the invention may be designed as short-acting, fast-releasing, or long-acting. Still further, compounds can be administered in a local rather than systemic means, such as administration (e.g., injection) as a sustained release formulation.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The pharmaceutical compositions of this invention may be in any orally acceptable dosage form, including capsules, tablets, aqueous suspensions or solutions. In the case of tablets for oral use, carriers that are commonly used include lactose and corn starch. Lubricating agents, such as magnesium stearate, are also typically added. For a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be in a topical form, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include mineral oil, sorbitan monostearate, polysorbate 60, cetyl esters, wax, cetyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative, such as benzylalkonium chloride. Alternatively, for ophthalmic uses, the pharmaceutical compositions may be formulated in an ointment, such as petrolatum.

The pharmaceutical compositions of this invention may also be administered by nasal aerosol or inhalation. Such compositions are prepared according to techniques known in the art of pharmaceutical formulation and may be prepared as solutions in saline, employing benzyl alcohol or other suitable preservatives, absorption promoters to enhance bioavailability, fluorocarbons and/or other conventional solubilizing or dispersing agents.

Any of the above dosage forms containing effective amounts are within the bounds of routine experimentation and within the scope of the invention. A therapeutically effective dose may vary depending upon the route of administration and dosage form. The preferred compound or compounds of the invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals. Effective doses can be extrapolated from dose-response curves derived from in vitro or animal model test systems. For example, an effective dose will typically be in the range of about 0.001 to about 1000 mg per kilogram body weight of the recipient per day (“mg/kg/day”), preferably about 0.01 to about 100 mg/kg/day, and more preferably about 0.1 to about 10 mg/kg/day.

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt % or about 20-80 wt %.

Besides those representative dosage forms described above, pharmaceutically acceptable excipients and carriers and dosage forms are generally known to those skilled in the art and are included in the invention. It should be understood that a specific dosage and treatment regimen for any particular patient will depend upon a variety of factors, including the activity of the specific compound employed, the age, body weight, general health, sex and diet of the patient, and the time of administration, rate of excretion, drug combination, judgment of the treating physician and severity of the particular disease being treated. The amount of active ingredient(s) will also depend upon the particular compound and other therapeutic agent, if present, in the composition.

### Methods of Use

The invention provides methods of inhibiting or decreasing Factor Xa activity as well as treating or ameliorating a Factor Xa associated state, symptom, disorder or disease in a patient in need thereof (e.g., human or non-human). “Treating” within the context of the invention means an alleviation of symptoms associated with a disorder or disease, or halt of further progression or worsening of those symptoms, or prevention or prophylaxis of the disease or disorder.

The term “mammal” includes organisms which express Factor Xa. Examples of mammals include mice, rats, cows, sheep, pigs, goats, horses, bears, monkeys, dogs, cats and, preferably, humans. Transgenic organisms which express Factor Xa are also included in this definition.

The inventive methods comprise administering an effective amount of a compound or composition described herein to a mammal or non-human animal. As used herein, “effective amount” of a compound or composition of the invention includes those amounts that antagonize or inhibit Factor Xa. An amount which antagonizes or inhibits Factor Xa is detectable, for example, by any assay capable of determining Factor Xa activity, including the one described below as an illustrative testing method. Effective amounts may also include those amounts which alleviate symptoms of a Factor Xa associated disorder treatable by inhibiting Factor Xa. Accordingly, “antagonists of Factor Xa” include compounds which interact with the Factor Xa and modulate, e.g., inhibit or decrease, the ability of a second compound, e.g., another Factor Xa ligand, to interact with the Factor Xa. The Factor Xa binding compounds are preferably antagonists of Factor Xa. The language “Factor Xa binding compound” (e.g., exhibits binding affinity to the receptor) includes those compounds which interact with Factor Xa resulting in modulation of the activity of Factor Xa. Factor Xa binding compounds may be identified using an in vitro (e.g., cell and non-cell based) or in vivo method. A description of an in vitro method is provided below.

The amount of compound present in the methods and compositions described herein should be sufficient to cause a detectable decrease in the severity of the disorder, as measured by any of the assays described in the examples. The amount of Factor Xa modulator needed will depend on the effectiveness of the modulator for the given cell type and the length of time required to treat the disorder. The effective amount is generally an amount described herein above. In certain embodiments, the compositions of this invention may further comprise another therapeutic agent. When a second agent is used, the second agent may be administered either as a separate dosage form or as part of a single dosage form with the compounds or compositions of this invention. While one or more of the inventive compounds can be used in an application of monotherapy to treat a disorder, disease or symptom, they also may be used in combination therapy, in which the use of an inventive compound or composition (therapeutic agent) is combined with the use of one or more other therapeutic agents for treating the same and/or other types of disorders, symptoms and diseases. Combination therapy includes administration of the two or more therapeutic agents concurrently or sequentially. The agents may be administered in any order. Alternatively, the multiple therapeutic agents can be combined into a single composition that can be administered to the patient. For instance, a single pharmaceutical composition could comprise the compound or pharmaceutically acceptable salt or solvate according to the any one of Formulas (I), (II), (Ia) and (Ib), another therapeutic agent (e.g., methotrexate) or a pharmaceutically acceptable salt or solvate thereof, and a pharmaceutically acceptable excipient or carrier.

The invention comprises a compound having any one of Formulas (I), (II), (Ia) and (Ib), a method for making an inventive compound, a method for making a pharmaceutical composition from at least one inventive compound and at least one pharmaceutically acceptable carrier or excipient, and a method of using one or more inventive compounds to treat a variety of disorders, symptoms and diseases (e.g., inflammatory, autoimmune, neurological, neurodegenerative, oncology and cardiovascular), such as RA, osteoarthritis, irritable bowel disease IBD, asthma, chronic obstructive pulmonary disease COPD and MS. The inventive compounds and their pharmaceutically acceptable salts and/or neutral compositions may be formulated together with a pharmaceutically acceptable excipient or carrier and the resulting composition may be administered in vivo to mammals, such as men, women and animals, to treat a variety of disorders, symptoms and diseases. Furthermore, the inventive compounds can be used to prepare a medicament that is useful for treating a variety of disorders, symptoms and diseases.

### Kits

Still another aspect of this invention is to provide a kit comprising separate containers in a single package, wherein the inventive pharmaceutical compounds, compositions and/or salts thereof are used in combination with pharmaceutically acceptable carriers to treat states, disorders, symptoms and diseases where Factor Xa plays a role.

### General Methods

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1967-2004, Volumes 1-22; Rodd's Chemistry of Carbon Compounds, Elsevier Science Publishers, 1989, Volumes 1-5 and Supplementals; and Organic Reactions, Wiley & Sons: New York, 2005, Volumes 1-65. The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C. to about 75° C.

Referring to the examples that follow, compounds of the present invention were synthesized using the methods described herein, or other methods, which are well known in the art.

The compounds and/or intermediates may be characterized by high performance liquid chromatography (HPLC) using a Waters Alliance chromatography system with a 2695 Separation Module (Milford, Mass.). The analytical columns may be C-18 SpeedROD RP-18E Columns from Merck KGaA (Darmstadt, Germany). Alternately, characterization may be performed using a Waters Unity (HPLC) system with Waters Acquity HPLC BEH C-18 2.1 mm×15 mm columns. A gradient elution may be used, typically starting with 5% acetonitrile/95% water and progressing to 95% acetonitrile over a period of 5 minutes for the Alliance system and 1 minute for the Acquity system. All solvents may contain 0.1% trifluoroacetic acid (TFA). Compounds may be detected by ultraviolet light (UV) absorption at either 220 or 254 nm. HPLC solvents may be from EMD Chemicals, Inc. (Gibbstown, N.J.). In some instances, purity may be assessed by thin layer chromatography (TLC) using glass backed silica gel plates, such as, for example, EMD Silica Gel 60 2.5 cm×7.5 cm plates. TLC results may be readily detected visually under ultraviolet light, or by employing well known iodine vapor and other various staining techniques.

Mass spectrometric analysis may be performed on one of two Agilent 1100 series LCMS instruments with acetonitrile/water as the mobile phase. One system using TFA as the modifier and measures in positive ion mode (reported as MH+, (M+1) or (M+H)+) and the other may use either formic acid or ammonium acetate and measures in both positive (reported as MH⁺, (M+1) or (M+H)⁺) and negative (reported as M−, (M−1) or (M−H)⁻) ion modes.

Nuclear magnetic resonance (NMR) analysis may be performed on some of the compounds with a Varian 400 MHz NMR (Palo Alto, Calif.). The spectral reference may be either TMS or the known chemical shift of the solvent.

The purity of some of compounds of the invention may be assessed by elemental analysis (Robertson Microlit, Madison N.J.).

Melting points may be determined on a Laboratory Devices MeI-Temp apparatus (Holliston, Mass.).

Preparative separations may be carried out using either an Sq16x or an Sg100c chromatography system and prepackaged silica gel columns all purchased from Teledyne Isco, (Lincoln, Nebr.). Alternately, compounds and intermediates may be purified by flash column chromatography using silica gel (230-400 mesh) packing material, or by HPLC using a C-18 reversed phase column. Typical solvents employed for the Isco systems and flash column chromatography may be dichloromethane, methanol, ethyl acetate, hexane, acetone, aqueous hydroxyamine and triethyl amine. Typical solvents employed for the reverse phase HPLC may be varying concentrations of acetonitrile and water with 0.1% trifluoroacetic acid.

### EXAMPLES

The following abbreviations are used throughout the Examples:
μL=microliter
μM=micromolar
AIBN=azobisisobutyronitrile
aq.=aqueous
Boc=tert-butoxycarbonyl
BOP=benzotriazol-1-yloxytris(dimethylamino)-phosphonium hexafluorophosphate
conc.=concentrated
DBU=1,8-diazabicyclo[5.4.0]undec-7-ene
DCM=dichloromethane
DIEA=diisopropylethyl amine
DMF=dimethyl formamide
DMSO=dimethyl sulfoxide
eq.=equivalent
EtOAc=ethyl acetate
g=gram
h or hr(s)=hour(s)
HATU=2-(1H-7-azabenzotriazol-1-yl)-1,1,3,3-tetramethyl uronium hexafluorophosphate
HOBt=N-hydroxybenzotriazole
HPLC=high pressure liquid chromatography
IC₅₀=the concentration of an inhibitor that is required for 50% inhibition of an enzyme in vitro
kg=kilogram
M=molar
m/z=mass to charge ratio
MeOH=methanol
mg=milligram
MHz=mega Hertz
min=minute
mL=milliliter
mM=millimolar
mm=millimeter
mmol=millimole
mOD/min=millioptical density units per minute
MS=Mass Spec
N=Normal
NaSMe=sodium methylthiolate
NBS=N-bromosuccinamide
nBuOH=n-butanol
ng=nanogram
nM=nanomolar
nm=nanometer
Pd(PPh₃)₄=tetrakis-(triphenylphosphine)-palladium
PEG=polyethylene glycol
pM=picomolar
PPh₃ or Ph₃P=triphenyl phosphine
PyBOP=(benzotriazol-1-yloxy)tripyrrolidinophosphonium Hexafluorophosphate
prep=preparative
Ra—Ni=Rainey Nickel
RT=room temperature
TEA=triethylamine
TFA=trifluoroacetic acid
TMSCl=trimethylsilyl chloride
TLC=thin layer chromatography

### Example 1

### 5-Chloro-N-((1-(2-(methylamino)-4-(2-oxopyridin-1(2H)-yl)phenyl)-1H-1,2,3-triazol-4-yl)methyl)thiophene-2-carboxamide (1)

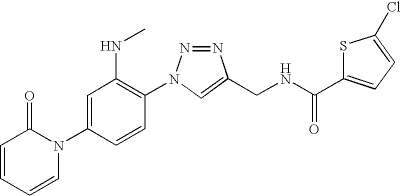

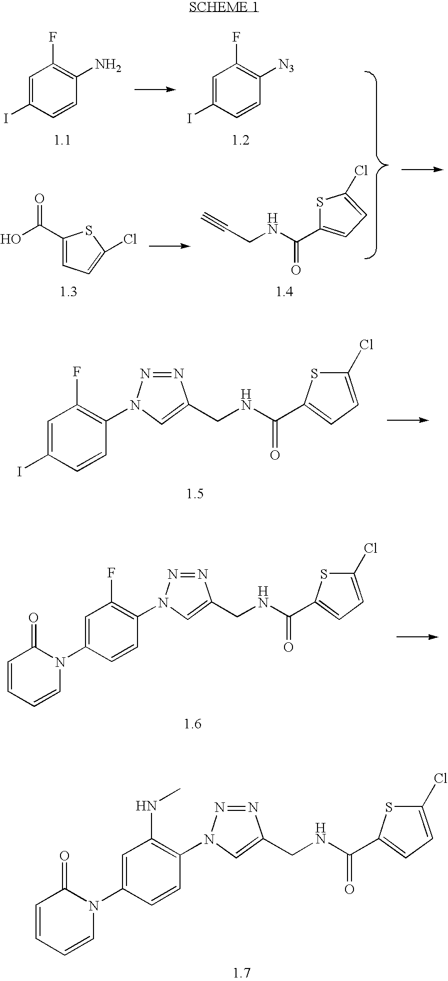

Step 1:

2-Fluoro-4-iodoaniline (1.1, 6.50 g, 27.4 mmol) was dissolved in 25 mL TFA and stirred in ice bath. Solid NaNO₂ (2.07 g, 30.1 mmol) was added in small portions. The resulting mixture was stirred for 30 min in ice bath. Sodium azide (1.87 g, 28.8 mmol) was dissolved in 10 mL water and chilled in ice bath. This cold solution was then added to the TFA solution in three portions. The mixture was stirred in ice bath for 1 hr and concentrated in vacuo to remove TFA. The residue was taken into 600 mL DCM and washed with water three times. The organic phase was dried using MgSO₄ and concentrated in vacuo to afford 1-azido-2-fluoro-4-iodobenzene 1.2 as a brownish waxy solid in >99% yield.

In the mean time, 5-chlorothiophene-2-carboxylic acid (1.3, 9.13 g, 56 mmol) was dissolved in 200 mL dry DCM along with 0.5 mL dry DMF. To the vigorously stirred solution was carefully added oxalyl chlororide (14.7 mL, 169 mmol) dropwise. The resulting solution was stirred for 3 hrs at RT and then concentrated in vacuo. The residue was pumped to dryness and then dissolved in 300 mL dry DCM. To this solution was added propargylamine (5.8 mL, 84 mmol) dropwise. The mixture was stirred at RT overnight during which time solid precipitated out. 600 mL hexane was added and the mixture was vigorously stirred for a few hours. The solid was collected by filtration and washed with hexane to give the product 1.4 (9.47 g, 85%) which was used directly without further purification. MS found for C₈H₆ClNOS as (M+H)+ 200.0, 202.0 (Cl pattern).

Step 2:

To a solution of the aryl azide 1.2 (27 mmol) and alkyne 1.4 (5.37 g, 27 mmol) in 500 mL dry methanol, were added DBU (4.00 mL, 54 mmol) and CuI (5.13 g, 27 mmol). The mixture was stirred at RT overnight. The mixture was then diluted with 1.0 liter acetonitrile and stirred vigorously for 1 hr. It was filtered through celite and the filtrate was concentrated and purified using flash column to give compound 1.5 (8.30 g, 67%). MS found for compound 1.5 C₁₄H₉ClFIN₄OS as (M+H)+ 463.0, 465.0 (Cl pattern).

Step 3:

To a solution of aryl iodide 1.5 (100 mg, 0.22 mmol) and 2-hydroxypyridine (42 mg, 0.44 mmol) in 5 mL dry DMSO in a sealed tube, were added 8-hydroxyquinoline (10 mg, 0.007 mmol), CuI (13 mg, 0.07 mmol) and Cs₂CO₃ (145 mg, 0.44 mmol). The mixture was stirred in 120° C. bath overnight. It was then filtered and the filtrate was directly subjected to reverse phase preparative HPLC to isolate compound 1.6 (66 mg) as a white powder in 68% yield after lyophilization. MS found for C₁₉H₁₃ClFN₅O₂S as (M+H)+ 430.0, 432.0 (Cl pattern).

Step 4:

To a solution of compound 1.6 (66 mg, 0.15 mmol) in 1 mL anhydrous DMSO in a sealed tube, was added methylamine (2.0 M in THF, 4 mL, 8.0 mmol). The mixture was stirred in 125° C. bath overnight. It was cooled to RT, concentrated in vacuo and directly subjected to reverse phase HPLC to isolate the title compound as a white powder after lyophilization. MS found for C₂₀H₁₇ClN₆O₂S (M+H)+ 441.1, 443.1 (Cl pattern).

### Example 2

### 5-Chloro-N-((1-(2-(dimethylamino)-4-(2-oxopyridin-1(2H)-yl)phenyl)-1H-1,2,3-triazol-4-yl)methyl)thiophene-2-carboxamide (2)

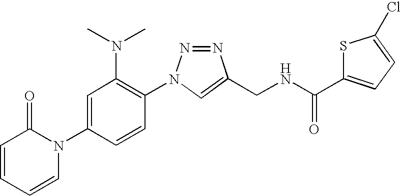

To a solution of compound 1.6 (55 mg, 0.13 mmol) in 2 mL anhydrous DMSO in a sealed tube, was added dimethylamine (2.0 M in MeOH, 2 mL, 4.0 mmol). The mixture was stirred in 125° C. bath overnight. It was cooled to RT, concentrated in vacuo and directly subjected to reverse phase HPLC to isolate the title compound as a white powder after lyophilization. MS found for C₂₁H₁₉ClN₆O₂S (M+H)+ 455.1, 457.1 (Cl pattern).

### Example 3

### 5-Chloro-N-((1-(4-(2-oxopyridin-1(2H)-yl)-2-(pyrrolidin-1-yl)phenyl)-1H-1,2,3-triazol-4-yl)methyl)thiophene-2-carboxamide (3)

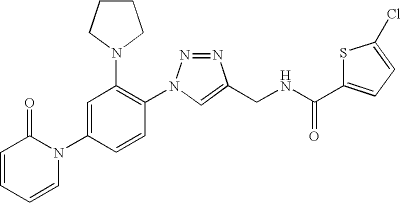

To a solution of compound 1.6 (100 mg, 0.23 mmol) in 1 mL anhydrous DMSO in a sealed tube was added 1 mL pyrrolidine. The mixture was stirred at 140° C. overnight. It was cooled to RT, concentrated in vacuo and directly subjected to reverse phase HPLC to isolate the title compound as a white powder after lyophilization. MS found for C₂₃H₂₁ClN₆O₂S (M+H)+ 481.1, 483.1 (Cl pattern).

### Example 4

### 5-Chloro-N-((1-(4-(2-oxopyridin-1(2H)-yl)-2-(piperidin-1-yl)phenyl)-1H-1,2,3-triazol-4-yl)methyl)thiophene-2-carboxamide (4)

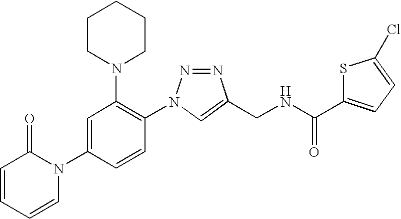

To a solution of compound 1.6 (100 mg, 0.23 mmol) in 1 mL anhydrous DMSO in a sealed tube, was added 1 mL piperidine. The mixture was stirred at 140° C. overnight. It was cooled to RT, concentrated in vacuo and directly subjected to reverse phase HPLC to isolate the title compound as a white powder after lyophilization. MS found for C₂₄H₂₃ClN₆O₂S (M+H)+ 495.1, 497.1 (Cl pattern).

### Example 5

### 5-Chloro-N-((1-(2-morpholino-4-(2-oxopyridin-1(2H)-yl)phenyl)-1H-1,2,3-triazol-4-yl)methyl)thiophene-2-carboxamide (5)

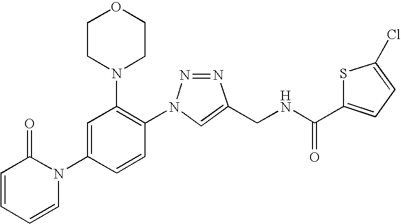

To a solution of compound 1.6 (100 mg, 0.23 mmol) in 1 mL anhydrous DMSO in a sealed tube, was added 1 mL morpholine. The mixture was stirred in 140° C. bath overnight. It was cooled to RT, concentrated in vacuo and directly subjected to reverse phase HPLC to isolate the title compound as a white powder after lyophilization. MS found for C₂₃H₂₀ClN₆O₃S (M+H)+ 497.1, 499.1 (Cl pattern).

### Example 6

### 5-Chloro-N-((1-(2-(3-oxopiperazin-1-yl)-4-(2-oxopyridin-1(2H)-yl)phenyl)-1H-1,2,3-triazol-4-yl)methyl)thiophene-2-carboxamide (6)

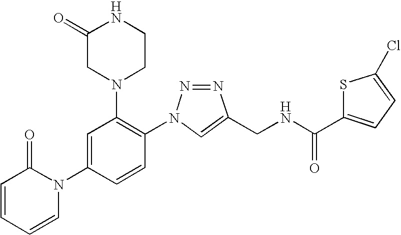

To a solution of compound 1.6 (100 mg, 0.23 mmol) in 2 mL anhydrous DMSO in a sealed tube was added 2-oxopiperazine (1.15 g, 11.5 mmol). The mixture was stirred in 140° C. bath for overnight. It was cooled to RT, and directly subjected to reverse phase HPLC to isolate the title compound as a white powder after lyophilization. MS found for C₂₃H₂₀ClN₇O₃S (M+H)+ 510.1, 512.1.

### Example 7

### 5-Chloro-N-((1-(2-(4-methyl-3-oxopiperazin-1-yl)-4-(2-oxopyridin-1(2H)-yl)phenyl)-1H-1,2,3-triazol-4-yl)methyl)thiophene-2-carboxamide (7)

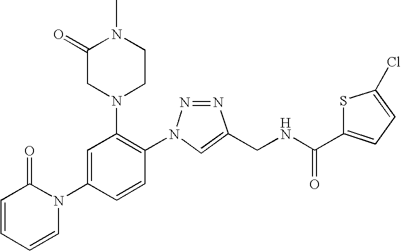

The title compound was prepared by a similar procedure as described in Example 6. MS found for C₂₄H₂₂ClN₇O₃S (M+H)+ 524.1, 526.1 (Cl pattern).

### Example 8

### 5-Chloro-N-((1-(2-(4-ethyl-3-oxopiperazin-1-yl)-4-(2-oxopyridin-1(2H)-yl)phenyl)-1H-1,2,3-triazol-4-yl)methyl)thiophene-2-carboxamide (8)

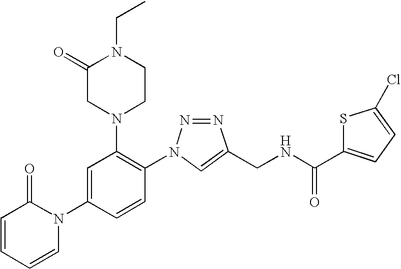

The title compound was prepared by a similar procedure as described in Example 7. MS found for C₂₅H₂₄ClN₇O₃S (M+H)+ 538.1, 540.1 (Cl pattern).

### Example 9

### 5-Chloro-N-((1-(2-(4-isopropyl-3-oxopiperazin-1-yl)-4-(2-oxopyridin-1(2H)-yl)phenyl)-1H-1,2,3-triazol-4-yl)methyl)thiophene-2-carboxamide (9)

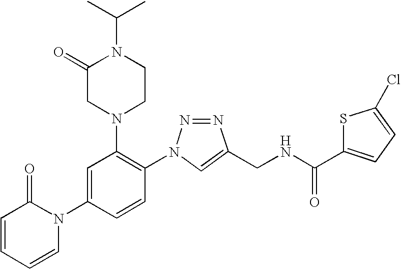

The title compound was prepared by a similar procedure as described in Example 7. MS found for C₂₆H₂₆ClN₇O₃S (M+H)+ 552.1, 554.1 (Cl pattern).

### Example 10

### 5-Chloro-N-((1-(4-(2-oxopyridin-1(2H)-yl)-2-thiomorpholinophenyl)-1H-1,2,3-triazol-4-yl)methyl)thiophene-2-carboxamide (10)

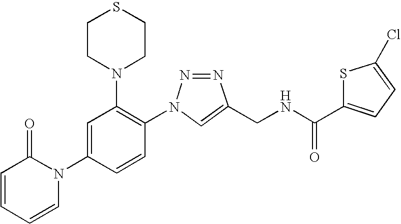

To a solution of compound 1.6 (100 mg, 0.23 mmol) in 1 mL anhydrous DMSO in a sealed tube was added 1 mL thiomorpholine. The mixture was stirred in 140° C. bath for overnight. It was cooled to RT, concentrated in vacuo and directly subjected to reverse phase HPLC to isolate the title compound as a white powder after lyophilization. MS found for C₂₃H₂₁ClN₆O₂S₂ (M+H)+ 513.1, 515.1 (Cl pattern).

### Example 11

### 5-Chloro-N-((1-(4-(2-oxopyridin-1(2H)-yl)-2-((1-oxo-)thiomorpholino)phenyl)-1H-1,2,3-triazol-4-yl)methyl)thiophene-2-carboxamide (11)

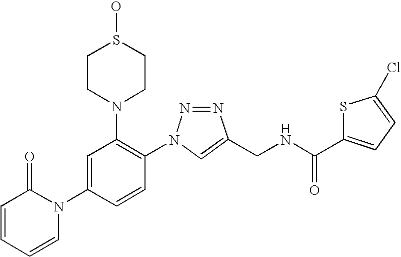

To a solution of 5-chloro-N-((1-(4-(2-oxopyridin-1(2H)-yl)-2-thiomorpholinophenyl)-1H-1,2,3-triazol-4-yl)methyl)thiophene-2-carboxamide (19 mg, 0.037 mmol, prepared as shown in Example 10) in 8 mL methanol and 4 mL water was added oxone (23 mg, 0.037 mmol). The mixture was stirred at RT for 10 min and directly subjected to reverse phase HPLC to isolate the title compound as a white powder after lyophilization. MS found for C₂₃H₂₁ClN₆O₃S₂ (M+H)+ 529.1, 531.1 (Cl pattern).

### Example 12

### 5-Chloro-N-((1-(4-(2-oxopyridin-1(2H)-yl)-2-((1,1-dioxo-)thiomorpholino)phenyl)-1H-1,2,3-triazol-4-yl)methyl)thiophene-2-carboxamide (12)

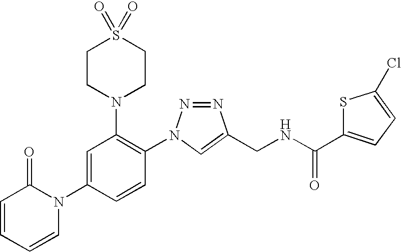

To a solution of 5-chloro-N-((1-(4-(2-oxopyridin-1(2H)-yl)-2-thiomorpholinophenyl)-1H-1,2,3-triazol-4-yl)methyl)thiophene-2-carboxamide (19 mg, 0.037 mmol, prepared as shown in Example 10) in 6 mL methanol and 3 mL water was added oxone (192 mg, 0.31 mmol). The mixture was stirred at RT for 1 hr and directly subjected to reverse phase HPLC to isolate the title compound as a white powder after lyophilization. MS found for C₂₃H₂₁ClN₆O₄S₂ (M+H)+ 545.1, 547.1 (Cl pattern).

### Example 13

### N-((1-(2-(4-Acetylpiperazin-1-yl)-4-(2-oxopyridin-1(2H)-yl)phenyl)-1H-1,2,3-triazol-4-yl)methyl)-5-chlorothiophene-2-carboxamide (13)

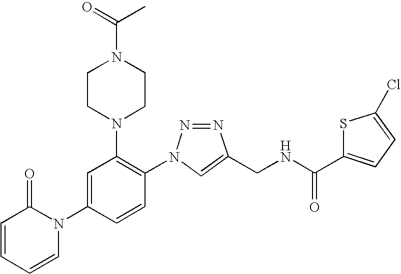

5-Chloro-N-((1-(4-(2-oxopyridin-1(2H)-yl)-2-(piperazin-1-yl)phenyl)-1H-1,2,3-triazol-4-yl)methyl)thiophene-2-carboxamide (15 mg, 0.003 mmol, prepared using a procedure similar to that described in Example 1) was dissolved in 1.5 mL anhydrous DMSO. To it was added 80 μL pyridine and 60 μL acetyl chloride. The mixture was stirred at RT for overnight and directly subjected to reverse phase HPLC to isolate the title compound as a white powder after lyophilization. MS found for C₂₅H₂₄ClN₇O₃S (M+H)+ 538.1, 540.1 (Cl pattern).

### Example 14

### 5-Chloro-N-((1-(2-(4-(1-iminoethyl)piperazin-1-yl)-4-(2-oxopyridin-1(2H)-yl)phenyl)-1H-1,2,3-triazol-4-yl)methyl)thiophene-2-carboxamide (14)

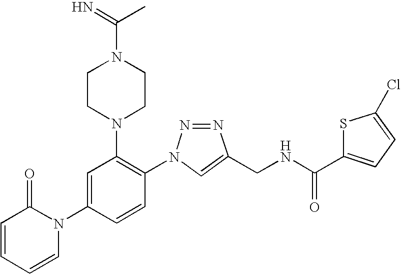

5-Chloro-N-((1-(4-(2-oxopyridin-1(2H)-yl)-2-(piperazin-1-yl)phenyl)-1H-1,2,3-triazol-4-yl)methyl)thiophene-2-carboxamide (15 mg, 0.003 mmol) was dissolved in 2 mL anhydrous methanol. To it was added 27 μL DIEA (0.15 mmol) and 20 mg ethyl acetimidate hydrochloride (0.15 mmol). The mixture was stirred in 100° C. in a sealed tube for overnight and directly subjected to reverse phase HPLC to isolate the title compound as a white powder after lyophilization. MS found for C₂₅H₂₅ClN₈O₂S (M+H)+ 537.1, 539.1 (Cl pattern).

### Example 15

### 4-(2-(4-((2-Chlorothiophene-5-carboxamido)methyl)-1H-1,2,3-triazol-1-yl)-5-(2-oxopyridin-1(2H)-yl)phenyl)piperazine-1-carboxamide (15)

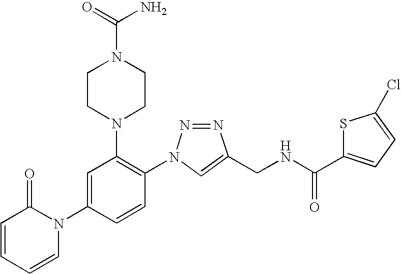

5-Chloro-N-((1-(4-(2-oxopyridin-1(2H)-yl)-2-(piperazin-1-yl)phenyl)-1H-1,2,3-triazol-4-yl)methyl)thiophene-2-carboxamide (50 mg, 0.1 mmol) was dissolved in 3 mL water and 0.5 mL DMSO. To it was added KOCN (41 mg, 0.5 mmol). The mixture was stirred at RT for 2 days and directly subjected to reverse phase HPLC to isolate the title compound as a white powder after lyophilization. MS found for C₂₄H₂₃ClN₈O₃S (M+H)+ 539.1, 541.1 (Cl pattern).

### Example 16

### 5-Chloro-N-((1-(2-(4-(dimethylamino)piperidin-1-yl)-4-(2-oxopyridin-1(2H)-yl)phenyl)-1H-1,2,3-triazol-4-yl)methyl)thiophene-2-carboxamide (16)

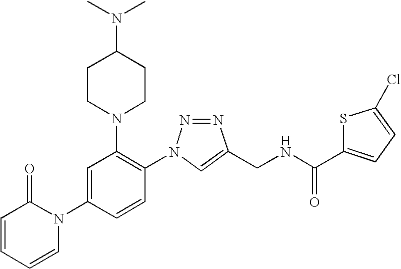

The title compound was prepared by a similar procedure as described in Example 5. MS found for C₂₆H₂₈ClN₇O₂S (M+H)+ 538.1, 540.1 (Cl pattern).

### Example 17

### N-((1-(2-(4-Aminopiperidin-1-yl)-4-(2-oxopyridin-1(2H)-yl)phenyl)-1H-1,2,3-triazol-4-yl)methyl)-5-chlorothiophene-2-carboxamide (17)

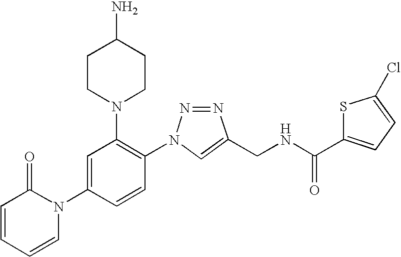

Compound 1.6 (200 mg, 0.47 mmol) was dissolved in 4 mL anhydrous DMSO in a sealed tube. 4-N-Boc-aminopyridine (2.82 g, 14.1 mmol) was added. The mixture was stirred in 120° C. bath for 24 hrs. It was cooled and diluted with 200 mL ethyl acetate. It was washed with brine three times, dried and concentrated in vacuo. The residue was then treated with 10 mL DCM and 10 mL TFA at RT for 1 hr. The mixture was then concentrated in vacuo and purified by reverse phase preparative HPLC to isolate the title compound. MS found for C₂₄H₂₄ClN₇O₂S (M+H)+ 510.1, 512.1 (Cl pattern).

### Example 18

### N-((1-(2-(4-Acetamidopiperidin-1-yl)-4-(2-oxopyridin-1(2H)-yl)phenyl)-1H-1,2,3-triazol-4-yl)methyl)-5-chlorothiophene-2-carboxamide (18)

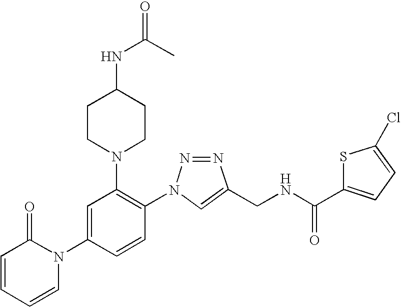

The title compound was prepared from N-((1-(2-(4-aminopiperidin-1-yl)-4-(2-oxopyridin-1(2H)-yl)phenyl)-1H-1,2,3-triazol-4-yl)methyl)-5-chlorothiophene-2-carboxamide by a similar procedure as described in Example 13. MS found for C₂₆H₂₆ClN₇O₃S (M+H)+ 552.1, 554.1 (Cl pattern).

### Example 19

### N-((1-(2-(4-Acetamidinopiperidin-1-yl)-4-(2-oxopyridin-1(2H)-yl)phenyl)-1H-1,2,3-triazol-4-yl)methyl)-5-chlorothiophene-2-carboxamide (19)

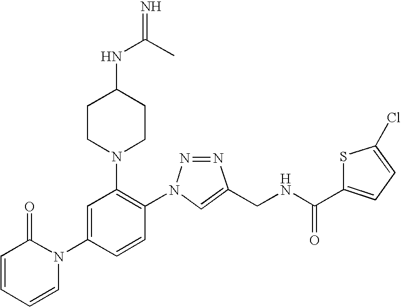

The title compound was prepared from N-((1-(2-(4-aminopiperidin-1-yl)-4-(2-oxopyridin-1(2H)-yl)phenyl)-1H-1,2,3-triazol-4-yl)methyl)-5-chlorothiophene-2-carboxamide by a similar procedure as described in Example 14. MS found for C₂₆H₂₇ClN₈O₂S (M+H)+ 551.1, 553.1 (Cl pattern).

### Example 20

### 1-(1-(2-(4-((2-Chlorothiophene-5-carboxamido)methyl)-1H-1,2,3-triazol-1-yl)-5-(2-oxopyridin-1(2H)-yl)phenyl)piperidin-4-yl)urea (20)

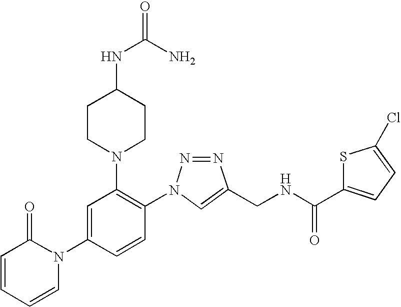

The title compound was prepared from N-((1-(2-(4-aminopiperidin-1-yl)-4-(2-oxopyridin-1(2H)-yl)phenyl)-1H-1,2,3-triazol-4-yl)methyl)-5-chlorothiophene-2-carboxamide by a similar procedure as described in Example 15. MS found for C₂₅H₂₅ClN₈O₃S (M+H)+ 553.1, 555.1 (Cl pattern).

### Example 21

### 5-Chloro-N-((1-(2-(4-hydroxypiperidin-1-yl)-4-(2-oxopyridin-1(2H)-yl)phenyl)-1H-1,2,3-triazol-4-yl)methyl)thiophene-2-carboxamide (21)

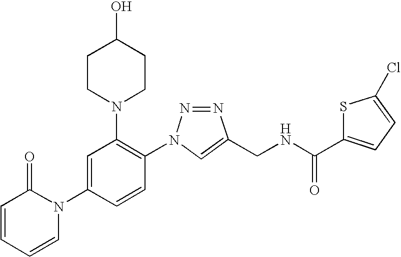

Compound 1.6 (100 mg, 0.23 mmol) was dissolved in 2 mL anhydrous DMSO in a sealed tube. 4-Hydroxypiperidine (0.95 g, 9.3 mmol) was added. The mixture was stirred in 130° C. bath for 16 hrs. The mixture was directly subjected to reverse phase preparative HPLC to isolated the title compound. MS found for C₂₄H₂₃ClN₆O₃S (M+H)+ 511.1, 513.1 (Cl pattern).

### Example 22

### 5-Chloro-N-((1-(2-((R)-3-hydroxypiperidin-1-yl)-4-(2-oxopyridin-1(2H)-yl)phenyl)-1H-1,2,3-triazol-4-yl)methyl)thiophene-2-carboxamide (22)

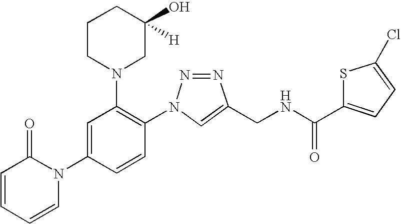

To a solution of (R)-3-hydroxypiperidine hydrochloride (762 mg, 5.6 mmol) in 2 mL anhydrous DMSO in a sealable tube was added sodium hydride (60% in mineral oil, 224 mg, 5.6 mmol). The mixture was stirred at RT for 30 min before compound 1.6 (80 mg, 0.18 mmol) was added. The tube was then sealed and stirred in 125° C. bath for 18 hrs. The mixture was directly subjected to reverse phase preparative HPLC to isolated the title compound. MS found for C₂₄H₂₃ClN₆O₃S (M+H)+ 511.1, 513.1 (Cl pattern).

### Example 23

### 5-Chloro-N-((1-(2-((S)-3-hydroxypiperidin-1-yl)-4-(2-oxopyridin-1(2H)-yl)phenyl)-1H-1,2,3-triazol-4-yl)methyl)thiophene-2-carboxamide (23)

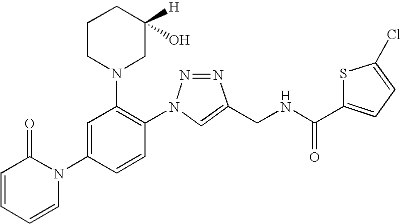

To a solution of (S)-3-hydroxypiperidine hydrochloride (762 mg, 5.6 mmol) in 2 mL anhydrous DMSO in a sealable tube was added sodium hydride (60% in mineral oil, 224 mg, 5.6 mmol). The mixture was stirred at RT for 30 min before compound 1.6 (80 mg, 0.18 mmol) was added. The tube was then sealed and the mixture was stirred in 125° C. bath for 18 hrs. The mixture was directly subjected to reverse phase preparative HPLC to isolated the title compound. MS found for C₂₄H₂₃ClN₆O₃S (M+H)+ 511.1, 513.1 (Cl pattern).

### Example 24

### 1-(2-(4-((2-Chlorothiophene-5-carboxamido)methyl)-1H-1,2,3-triazol-1-yl)-5-(2-oxopyridin-1(2H)-yl)phenyl)piperidine-4-carboxylic acid (24)

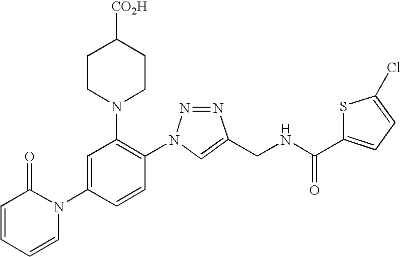

To a solution of compound 1.6 (200 mg, 0.47 mmol) in 2 mL anhydrous DMSO in a sealed tube was added 2 mL methyl isonipecotate. The mixture was heated in 140° C. bath for 20 hrs and concentrated in vacuo. The residue was then dissolved in 20 mL methanol. To it were added 10 mL water and 100 mg lithium hydroxide hydrate. The mixture was stirred at RT for 4 hrs and concentrated in vacuo. The residue was directly subjected to reverse phase preparative HPLC to isolated the title compound. MS found for C₂₅H₂₃ClN₆O₄S (M+H)+ 539.1, 541.1 (Cl pattern).

### Example 25

### 1-(2-(4-((2-Chlorothiophene-5-carboxamido)methyl)-1H-1,2,3-triazol-1-yl)-5-(2-oxopyridin-1(2H)-yl)phenyl)piperidine-4-carboxamide (25)

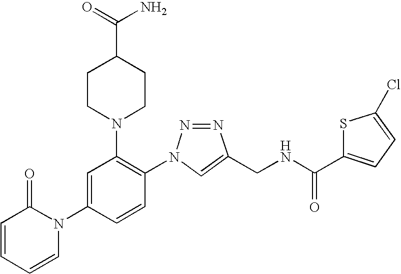

To a solution of compound 1.6 (100 mg, 0.23 mmol) in 2 mL anhydrous DMSO in a sealed tube was added 1.0 g isonipecotamide (7.75 mmol). The mixture was heated in 140° C. bath for 20 hrs and directly subjected to reverse phase preparative HPLC to isolated the title compound. MS found for C₂₅H₂₄ClN₇O₃S (M+H)+ 538.1, 540.1 (Cl pattern).

### Example 26

### 5-Chloro-N-((1-(2-(2-hydroxyethylamino)-4-(2-oxopyridin-1(2H)-yl)phenyl)-1H-1,2,3-triazol-4-yl)methyl)thiophene-2-carboxamide (26)

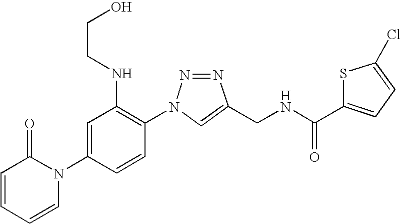

To a solution of compound 1.6 (65 mg, 0.15 mmol) in 1 mL anhydrous DMSO in a sealed tube was added 1 mL ethanolamine. The mixture was heated in 125° C. bath for 18 hrs and directly subjected to reverse phase preparative HPLC to isolated the title compound. MS found for C₂₁H₁₉ClN₆O₃S (M+H)+ 471.1, 473.1 (Cl pattern).

### Example 27

### 5-Chloro-N-((1-(2-(2-methoxyethylamino)-4-(2-oxopyridin-1(2H)-yl)phenyl)-1H-1,2,3-triazol-4-yl)methyl)thiophene-2-carboxamide (27)

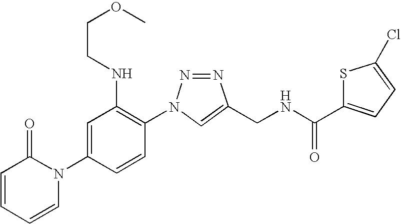

The title compound was prepared by a similar procedure as described in Example 26. MS found for C₂₂H₂₁ClN₆O₃S (M+H)+ 485.1, 487.1 (Cl pattern).

### Example 28

### 5-Chloro-N-((1-(2-((2-hydroxyethyl)(methyl)amino)-4-(2-oxopyridin-1(2H)-yl)phenyl)-1H-1,2,3-triazol-4-yl)methyl)thiophene-2-carboxamide (28)

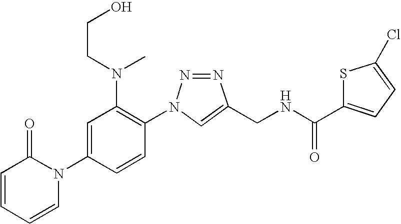

The title compound was prepared by a similar procedure as described in Example 26. MS found for C₂₂H₂₁ClN₆O₃S (M+H)+ 485.1, 487.1 (Cl pattern).

### Example 29

### 5-Chloro-N-((1-(2-((2-methoxyethyl)(methyl)amino)-4-(2-oxopyridin-1(2H)-yl)phenyl)-1H-1,2,3-triazol-4-yl)methyl)thiophene-2-carboxamide (29)

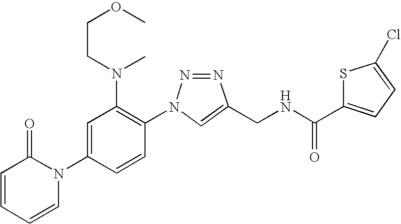

The title compound was prepared a similar procedure as described in Example 26. MS found for C₂₃H₂₃ClN₆O₃S (M+H)+ 499.1, 501.1 (Cl pattern).

### Example 30

### N-((1-(2-(2-Aminoethylamino)-4-(2-oxopyridin-1(2H)-yl)phenyl)-1H-1,2,3-triazol-4-yl)methyl)-5-chlorothiophene-2-carboxamide (30)

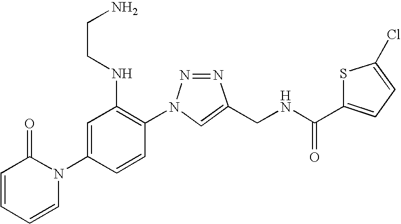

The title compound was prepared by a similar procedure as described in Example 26. MS found for C₂₁H₂₀ClN₇O₂S (M+H)+ 470.1, 472.1 (Cl pattern).

### Example 31

### 5-Chloro-N-((1-(2-(2-(dimethylamino)ethylamino)-4-(2-oxopyridin-1(2H)-yl)phenyl)-1H-1,2,3-triazol-4-yl)methyl)thiophene-2-carboxamide (31)

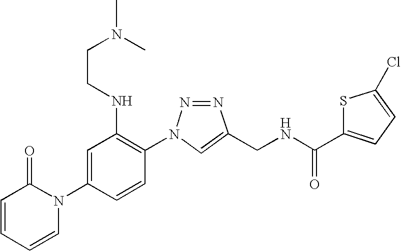

The title compound was prepared by a similar procedure as described in Example 26. MS found for C₂₃H₂₄ClN₇O₂S (M+H)+ 498.1, 500.1 (Cl pattern).

### Example 32

### 5-Chloro-N-((1-(2-((2-(dimethylamino)ethyl)(methyl)amino)-4-(2-oxopyridin-1(2H)-yl)phenyl)-1H-1,2,3-triazol-4-yl)methyl)thiophene-2-carboxamide (32)

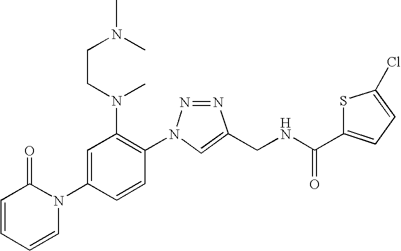

The title compound was prepared by a similar procedure as described in Example 26. MS found for C₂₄H₂₆ClN₇O₂S (M+H)+ 512.1, 514.1 (Cl pattern).

### Example 33

### 5-Chloro-N-((1-(2-(3-hydroxypropylamino)-4-(2-oxopyridin-1(2H)-yl)phenyl)-1H-1,2,3-triazol-4-yl)methyl)thiophene-2-carboxamide (33)

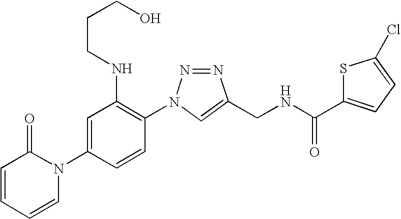

The title compound was prepared by a similar procedure as described in Example 26. MS found for C₂₂H₂₁ClN₆O₃S (M+H)+ 485.1, 487.1 (Cl pattern).

### Example 34

### 5-Chloro-N-((1-(2-(3-methoxypropylamino)-4-(2-oxopyridin-1(2H)-yl)phenyl)-1H-1,2,3-triazol-4-yl)methyl)thiophene-2-carboxamide (34)

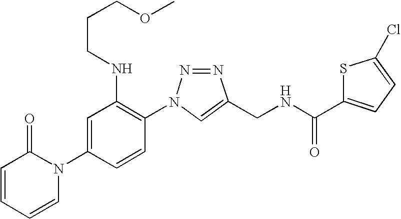

The title compound was prepared by a similar procedure as described in Example 26. MS found for C₂₃H₂₃ClN₆O₃S (M+H)+ 499.1, 501.1 (Cl pattern).

### Example 35

### 5-Chloro-N-((1-(2-((3-hydroxypropyl)(methyl)amino)-4-(2-oxopyridin-1(2H)-yl)phenyl)-1H-1,2,3-triazol-4-yl)methyl)thiophene-2-carboxamide (35)

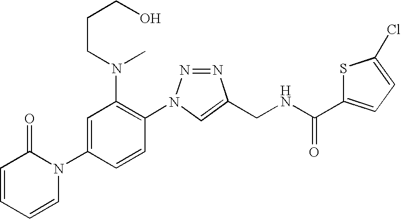

The title compound was prepared by a similar procedure as described in Example 26. MS found for C₂₃H₂₃ClN₆O₃S (M+H)+ 499.1, 501.1 (Cl pattern).

### Example 36

### 5-Chloro-N-((1-(2-((3-methoxypropyl)(methyl)amino)-4-(2-oxopyridin-1(2H)-yl)phenyl)-1H-1,2,3-triazol-4-yl)methyl)thiophene-2-carboxamide (36)

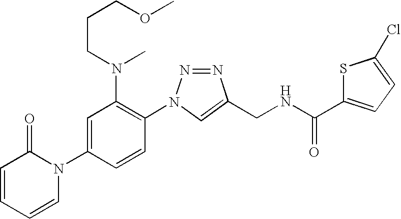

The title compound was prepared by a similar procedure as described in Example 26. MS found for C₂₄H₂₅ClN₆O₃S (M+H)+ 513.1, 515.1 (Cl pattern).

### Example 37

### N-((1-(2-(3-Aminopropylamino)-4-(2-oxopyridin-1(2H)-yl)phenyl)-1H-1,2,3-triazol-4-yl)methyl)-5-chlorothiophene-2-carboxamide (37)

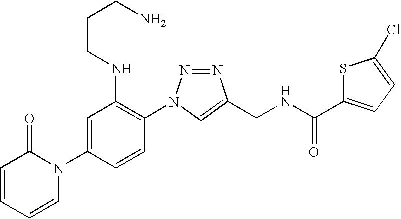

The title compound was prepared by a similar procedure as described in Example 26. MS found for C₂₂H₂₂ClN₇O₂S (M+H)+ 484.1, 486.1 (Cl pattern).

### Example 38

### 5-Chloro-N-((1-(2-(3-(dimethylamino)propylamino)-4-(2-oxopyridin-1(2H)-yl)phenyl)-1H-1,2,3-triazol-4-yl)methyl)thiophene-2-carboxamide (38)

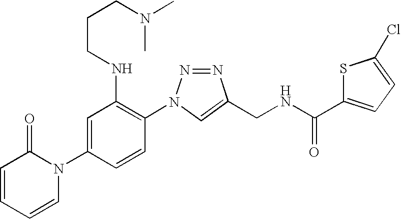

The title compound was prepared by a similar procedure as described in Example 26. MS found for C₂₄H₂₆ClN₇O₂S (M+H)+ 512.1, 514.1 (Cl pattern).

### Example 39

### 5-Chloro-N-((1-(2-((3-(dimethylamino)propyl)(methyl)amino)-4-(2-oxopyridin-1(2H)-yl)phenyl)-1H-1,2,3-triazol-4-yl)methyl)thiophene-2-carboxamide (39)

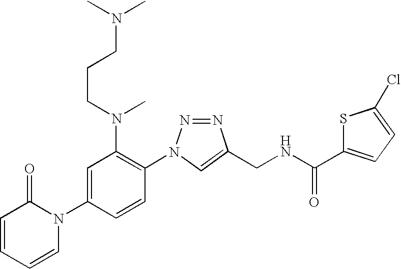

The title compound was prepared by a similar procedure as described in Example 26. MS found for C₂₅H₂₈ClN₇O₂S (M+H)+ 526.1, 528.1 (Cl pattern).

### Example 40

### 5-Chloro-N-((1-(2-(methyl(3-(methylamino)propyl)amino)-4-(2-oxopyridin-1(2H)-yl)phenyl)-1H-1,2,3-triazol-4-yl)methyl)thiophene-2-carboxamide (40)

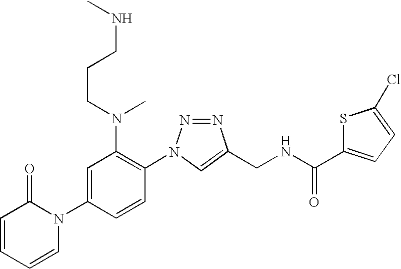

The title compound was prepared by a similar procedure as described in Example 26. MS found for C₂₄H₂₆ClN₇O₂S (M+H)+ 512.1, 514.1 (Cl pattern).

### Example 41

### 5-Chloro-N-((1-(2-(methyl(2-(methylamino)ethyl)amino)-4-(2-oxopyridin-1(2H)-yl)phenyl)-1H-1,2,3-triazol-4-yl)methyl)thiophene-2-carboxamide (41)

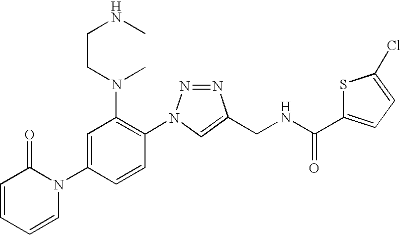

The title compound was prepared by a similar procedure as described in Example 26. MS found for C₂₃H₂₄ClN₇O₂S (M+H)+ 498.1, 500.1 (Cl pattern).

### Example 42

### N-((1-(2-(1H-Imidazol-1-yl)-4-(2-oxopyridin-1(2H)-yl)phenyl)-1H-1,2,3-triazol-4-yl)methyl)-5-chlorothiophene-2-carboxamide (42)

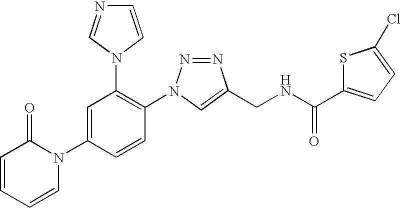

The title compound was prepared by a similar procedure as described in Example 26. MS found for C₂₂H₁₆ClN₇O₂S (M+H)+ 478.1, 480.1 (Cl pattern).

### Example 43

### 5-Chloro-N-((1-(4-(2-oxopyridin-1(2H)-yl)-2-(2-oxopyrrolidin-1-yl)phenyl)-1H-1,2,3-triazol-4-yl)methyl)thiophene-2-carboxamide (43)

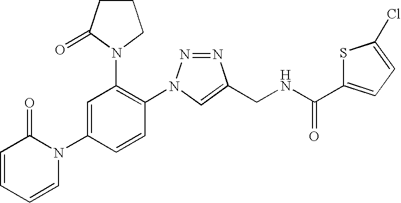

The title compound was prepared by a similar procedure as described in Example 26. MS found for C₂₃H₁₉ClN₆O₃S (M+H)+ 495.1.

### Example 44

### 5-Chloro-N-((1-(2-nitro-4-(2-oxopyridin-1(2H)-yl)phenyl)-1H-1,2,3-triazol-4-yl)methyl)thiophene-2-carboxamide (44)

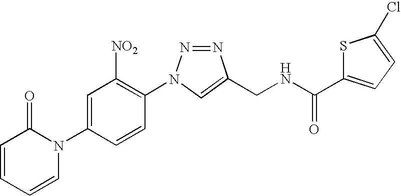

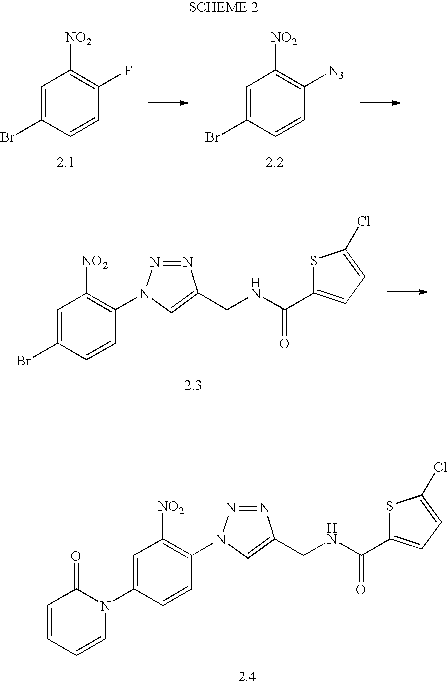

Step 1:

To a solution of 1-Bromo-4-fluoro-3-nitrobenzene (2.44 g, 11 mmol) in 40 mL DMSO was added sodium azide (1.44 g, 22 mmol). The mixture was stirred at RT for 10 min. It was diluted with 500 mL ethyl acetate and washed with brine three times. The organic phase was dried and concentrated in vacuo to afford compound 2.2 (3.00 g, 100%) cleanly.

Step 2:

Compound 2.2 (all from previous step) was dissolved in 100 mL methanol. To it were added compound 1.4 (3.3 g, 16.5 mmol), DBU (3.3 mL, 22 mmol) and CuI (4.18 g, 22 mmol). The mixture was stirred overnight. It was diluted with 400 mL ethyl acetate and washed with brine twice. The organic phase was dried, concentrated and purified using flash column to afford compound 2.3 (1.07 g, 22% for two steps). MS found for C₁₄H₉BrClN₅O₃S (M+H)+ 442.0, 444.0, 446.0.

Step 3:

Compound 2.3 (145 mg, 0.33 mmol) was dissolved in 5 mL dioxane and 1 mL DMSO. To it were added 2-hydroxypyridine (125 mg, 1.32 mmol), N,N′-dimethylethylenediamine (22 μL, 0.2 mmol) and K₃PO₄ (140 mg, 0.66 mmol). To it was then added CuI (32 mg, 0.17 mmol). The mixture was heated in 120° C. bath in a sealed tube for 16 hrs. The mixture was then concentrated in vacuo and directly subjected to reverse phase preparative HPLC to isolate the title compound 2.4. MS found for C₁₉H₁₃ClN₆O₄S (M+H)+ 457.0, 459.0 (Cl pattern).

### Example 45

### N-((1-(2-Amino-4-(2-oxopyridin-1(2H)-yl)phenyl)-1H-1,2,3-triazol-4-yl)methyl)-5-chlorothiophene-2-carboxamide (45)

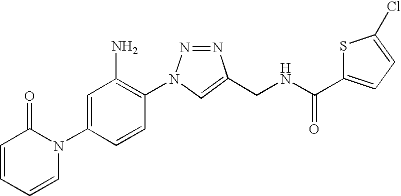

To a solution of compound 2.4 (6 mg, 0.013 mmol) in 2 mL acetic acid and 2 mL ethanol was added iron powder (5 mg, 0.08 mmol). The mixture was stirred in 100° C. bath for 30 min and directly subjected to reverse phase preparative HPLC to isolate the title compound. MS found for C₁₉H₁₅ClN₆O₂S (M+H)+ 427.1, 429.1 (Cl pattern).

### Example 46

### 1-(2-(4-((2-Chlorothiophene-5-carboxamido)methyl)-1H-1,2,3-triazol-1-yl)-5-(2-oxopyridin-1(2H)-yl)phenyl)urea (46)

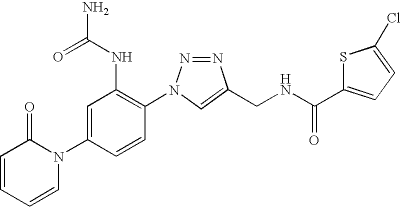

N-((1-(2-Amino-4-(2-oxopyridin-1(2H)-yl)phenyl)-1H-1,2,3-triazol-4-yl)methyl)-5-chlorothiophene-2-carboxamide (43 mg, 0.10 mmol) was dissolved in 2 mL water and 1 mL DMSO. To it was added KOCN (82 mg, 1.0 mmol). The mixture was stirred in 60° C. bath for 2 days and directly subjected to reverse phase preparative HPLC to isolate the title compound. MS found for C₂₀H₁₆ClN₇O₃S (M+H)+ 470.1, 472.1 (Cl pattern).

### Example 47

### N-((1-(2-Acetamido-4-(2-oxopyridin-1(2H)-yl)phenyl)-1H-1,2,3-triazol-4-yl)methyl)-5-chlorothiophene-2-carboxamide (47)

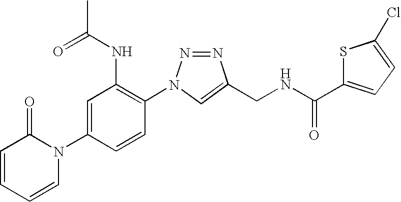

N-((1-(2-Amino-4-(2-oxopyridin-1(2H)-yl)phenyl)-1H-1,2,3-triazol-4-yl)methyl)-5-chlorothiophene-2-carboxamide (43 mg, 0.10 mmol) was dissolved in 2 mL anhydrous DMSO. To it was added 120 μL pyridine and 100 μL acetyl chloride. The mixture was in 60° C. bath for 2 days in a sealed tube and directly subjected to reverse phase preparative HPLC to isolate the title compound. MS found for C₂₁H₁₇ClN₆O₃S (M+H)+ 469.1, 471.1 (Cl pattern).

### Example 48

### N-(2-(4-((2-Chlorothiophene-5-carboxamido)methyl)-1H-1,2,3-triazol-1-yl)-5-(2-oxopyridin-1(2H)-yl)phenyl)isonicotinamide (48)

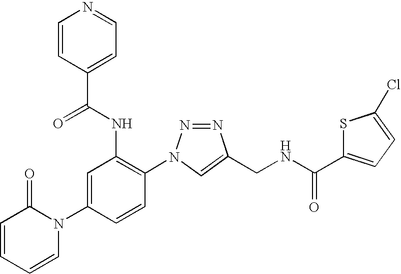

The title compound was prepared by a similar procedure as described in Example 47. MS found for C₂₅H₁₈ClN₇O₃S (M+H)+ 532.1, 534.1 (Cl pattern).

### Example 49

### N-(2-(4-((2-Chlorothiophene-5-carboxamido)methyl)-1H-1,2,3-triazol-1-yl)-5-(2-oxopyridin-1(2H)-yl)phenyl)nicotinamide (49)

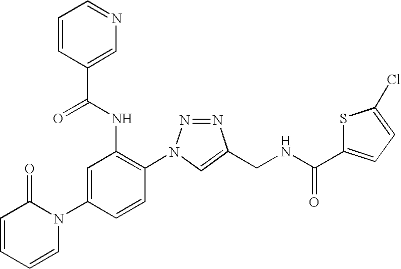

The title compound was prepared by a similar procedure as described in Example 47. MS found for C₂₅H₁₈ClN₇O₃S (M+H)+ 532.1, 534.1 (Cl pattern).

### Example 50

### 5-Chloro-N-((1-(2-(methylthio)-4-(2-oxopyridin-1(2H)-yl)phenyl)-1H-1,2,3-triazol-4-yl)methyl)thiophene-2-carboxamide (50)

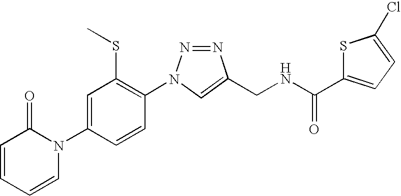

To a solution of compound 1.6 (690 mg, 1.6 mmol) was dissolved in 20 mL anhydrous DMSO was added sodium thiomethoxide (225 mg, 3.2 mmol). The mixture was stirred in 100° C. bath for 1 hr, and diluted with ethyl acetate. It was washed with brine three times, dried, concentrated and purified using flash column to afford the title compound (500 mg, 68% yield) as a white solid. MS found for C₂₀H₁₆ClN₅O₂S₂ (M+H)+ 458.0, 460.0 (Cl pattern).

### Example 51

### 5-Chloro-N-((1-(2-(methylsulfoxy)-4-(2-oxopyridin-1(2H)-yl)phenyl)-1H-1,2,3-triazol-4-yl)methyl)thiophene-2-carboxamide (51)

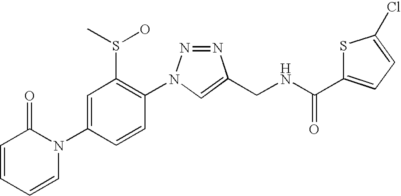

5-Chloro-N-((1-(2-(methylthio)-4-(2-oxopyridin-1(2H)-yl)phenyl)-1H-1,2,3-triazol-4-yl)methyl)thiophene-2-carboxamide (500 mg, 1.1 mmol) was dissolved in 20 mL methanol and 10 mL water. Oxone (677 mg, 1.1 mmol) was added in small portions. The mixture was stirred at RT for 45 min and directly subjected to reverse phase prep HPLC to isolate the title compound. MS found for C₂₀H₁₆ClN₅O₃S₂ (M+H)+ 474.0, 476.0 (Cl pattern).

### Example 52

### 5-Chloro-N-((1-(2-(methylsulfonyl)-4-(2-oxopyridin-1(2H)-yl)phenyl)-1H-1,2,3-triazol-4-yl)methyl)thiophene-2-carboxamide (52)

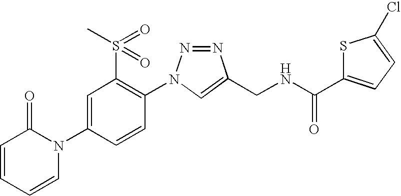

5-Chloro-N-((1-(2-(methylsulfoxy)-4-(2-oxopyridin-1(2H)-yl)phenyl)-1H-1,2,3-triazol-4-yl)methyl)thiophene-2-carboxamide (100 mg, 0.21 mmol) was dissolved in 10 mL methanol and 5 mL water. Oxone (270 mg, 0.44 mmol) was added in small portions. The mixture was stirred at RT for overnight and directly subjected to reverse phase prep HPLC to isolate the title compound. MS found for C₂₀H₁₆ClN₅O₄S₂ (M+H)+ 490.0, 492.0 (Cl pattern).

### Example 53

### N-((1-(2-(2-Aminoethylthio)-4-(2-oxopyridin-1(2H)-yl)phenyl)-1H-1,2,3-triazol-4-yl)methyl)-5-chlorothiophene-2-carboxamide (53)

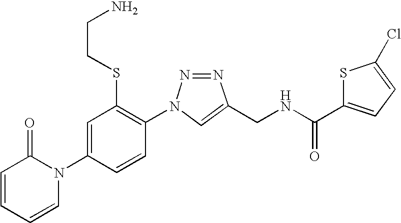

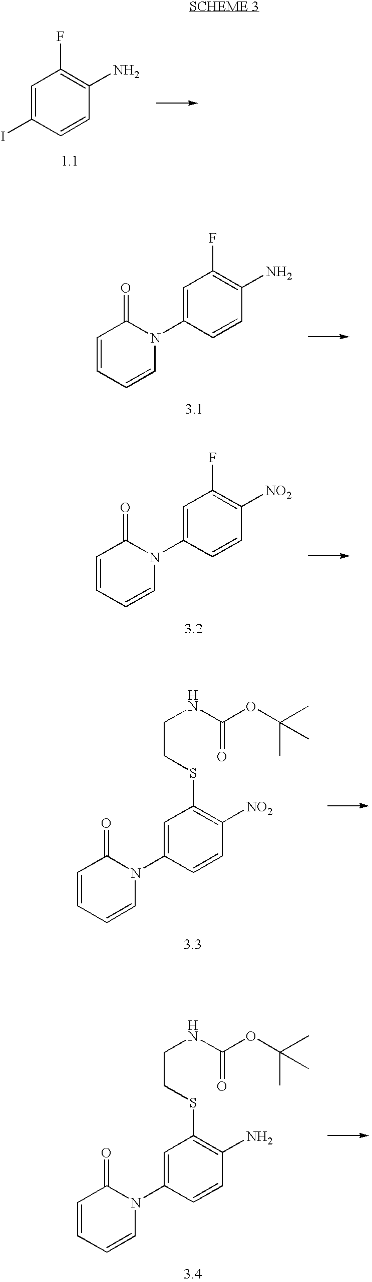

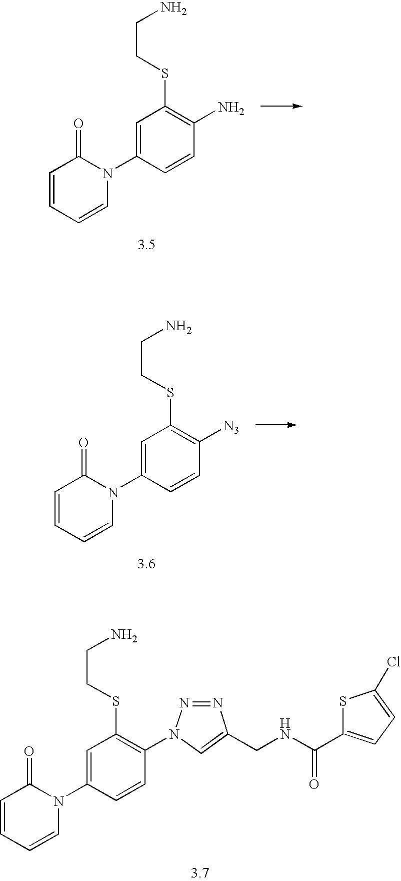

Step 1:

The mixture of 2-fluoro-4-iodoaniline 1.1 (23.7 g, 100 mmol), 2-hydroxypyridine (19.0 g, 200 mmol), 8-hydroxyquinoline (2.9 g, 20 mmol), CuI (3.8 g, 20 mmol) and cesium carbonate (65.2 g, 200 mmol) in 80 mL DMSO and 120 mL dioxane was stirred in 120° C. bath overnight. It was then concentrated in vacuo to remove dioxane. To the residue was added 300 mL brine. It was extracted with chloroform 300 mL six times. The organic extracts were combined, filtered, dried, and concentrated in vacuo to remove three-fourth of the chloroform. The resulting solid was collected by filtration, rinsed with cold DCM, and dried in vacuo to give compound 3.1 in 72% yield (14.8 g). MS found for C₁₁H₉FN₂O (M+H)+ 205.1.

Step 2:

To a stirring solution of trifluoroacetic anhydride (51 mL, 367 mmol) in 200 mL dry DCM in a 1 liter flask in ice bath was added hydrogen peroxide (50% water solution, 23 mL, 367 mmol) dropwise. The mixture was stirred in ice bath for 90 min. Compound 3.1 (7.5 g, 36.7 mmol) was then added in small portions over 10 min. The mixture was stirred overnight, allowed to warm up to RT naturally. To the reaction mixture was then added 300 mL brine. The organic phase was separated. The aqueous phase was extracted with chloroform 400 mL twice. The organic extracts were combined, dried and concentrated in vacuo to give compound 3.2 (9.1 g, 100%). MS found for C₁₁H₇FN₂O₃ (M+H)+ 235.0.

Step 3:

A mixture of compound 3.2 (0.93 g, 4.0 mmol), tert-butyl 2-mercaptoethylcarbamate (1.35 mL, 8.0 mmol) and DIEA (1.4 mL, 8.0 mmol) in 20 mL DMSO was stirred in 90° C. bath for 1 hr. It was diluted with ethyl acetate and washed with saturated ammonium chloride twice and brine once. The organic phase was dried and concentrated in vacuo to give crude product 3.3. Crude compound 3.3 was then dissolved in 80 mL ethanol and 40 mL acetic acid. To it was added iron powder (1.34 g, 24 mmol). The mixture was stirred in 100° C. bath for 90 min and diluted with acetonitrile. The mixture was filtered through a celite bed, and concentrated in vacuo to yield crude product 3.4. Crude compound 3.4 was then treated with 10 mL DCM and 10 mL TFA for 1 hr. The mixture was concentrated in vacuo and subjected to reverse phase preparative HPLC to isolate product 3.5 (0.7 g). MS found for C₁₃H₁₅N₃OS (M+H)+ 262.1.

Step 4:

Compound 3.5 (0.7 g, 1.4 mmol) was dissolved in 10 mL TFA and stirred in ice bath. To it was added powder NaNO₂ (110 mg, 1.5 mmol) in small portions. The mixture was stirred for 40 min in ice bath. and chilled in ice bath. To the reaction mixture was added a chilled solution of sodium azide (190 mg, 2.8 mmol) in 2 mL water this cold. The mixture was stirred in ice bath for 1 hr and at RT for 30 min, and then directly subjected to reverse phase preparative HPLC to isolate compound 3.6 (540 mg). MS found for C₁₃H₁₃N₅OS (M+H)+ 288.1.

Step 5:

Compound 3.6 (540 mg, 1.3 mmol) was dissolved in 20 mL methanol. To it were added compound 1.4 (400 mg, 2.0 mmol), DBU (0.6 mL, 4.0 mmol) and CuI (513 mg, 2.7 mmol). The mixture was stirred at RT overnight, diluted with acetonitrile, filtered through a celite bed, concentrated and subjected to reverse phase preparative HPLC to isolate the title compound 3.7. MS found for C₂₁H₁₉ClN₆O₂S₂ (M+H)+ 487.1, 489.1 (Cl pattern).

### Example 54

### N-((1-(2-(2-Aminoethylsulfoxy)-4-(2-oxopyridin-1(2H)-yl)phenyl)-1H-1,2,3-triazol-4-yl)methyl)-5-chlorothiophene-2-carboxamide (54)

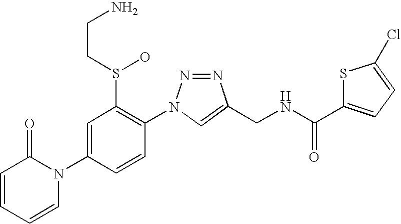

The title compound was prepared from compound 3.7 using conditions similar to those described in Example 51 MS found for C₂₁H₁₉ClN₆O₃S₂ (M+H)+ 503.1, 505.1 (Cl pattern).

### Example 55

### N-((1-(2-(2-Aminoethylsulfonyl)-4-(2-oxopyridin-1(2H)-yl)phenyl)-1H-1,2,3-triazol-4-yl)methyl)-5-chlorothiophene-2-carboxamide (55)

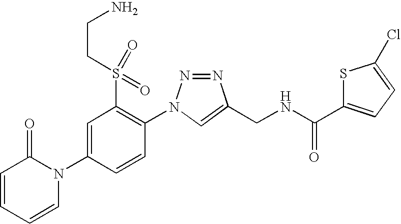

The title compound was prepared from compound 3.7 using the same procedure described for Example 52. MS found for C₂₁H₁₉ClN₆O₄S₂ (M+H)+ 519.1, 521.1 (Cl pattern).

### Example 56

### 5-Chloro-N-((1-(2-methoxy-4-(2-oxopyridin-1(2H)-yl)phenyl)-1H-1,2,3-triazol-4-yl)methyl)thiophene-2-carboxamide (56)

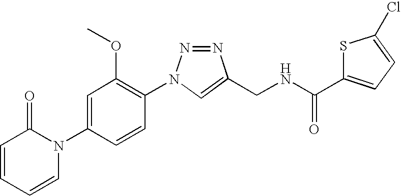

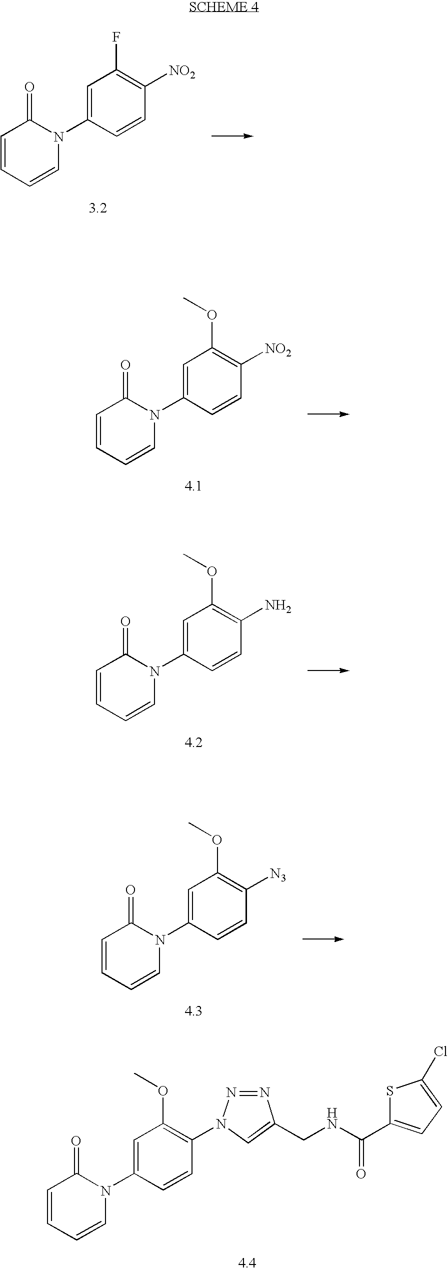

Step 1:

To a solution of compound 3.2 (300 mg, 1.28 mmol) in 10 mL DMSO was added sodium methoxide (280 mg, 5.12 mmol). The mixture was stirred in 100° C. bath for 10 min and diluted with saturated ammonium chloride aq. solution. It was extracted with ethyl acetate. The organic extracts were combined, dried, concentrated in vacuo and purified using reverse phase preparative HPLC to afford compound 4.1 (55%). MS found for C₁₂H₁₀N₂O₄ (M+H)+ 247.1.

Step 2:

Compound 4.1 (48 mg, 0.2 mmol) was dissolved in 6 mL ethanol and 3 mL acetic acid and treated with iron powder (68 mg, 1.2 mmol) at 100° C. for 30 min. The mixture was filtered and subjected to reverse phase preparative HPLC to isolate compound 4.2 (70%). MS found for C₁₂H₁₂N₂O₂ (M+H)+ 217.1.

Step 3:

Compound 4.2 (30 mg, 0.14 mmol) was dissolved in 2 mL TFA and stirred in ice bath. To it was added sodium nitrite (12 mg, 0.16 mmol). The mixture was stirred for 40 min. To it was then added an ice-cold solution of sodium azide (18 mg) in 1 mL water. The mixture was stirred in ice bath for 2 hr and subjected to reverse phase preparative HPLC to give compound 4.3 (40%). MS found for C₁₂H₁₀N₄O₂ (M+H)+ 243.1.

Step 4:

Compound 4.3 (10 mg, 0.04 mmol) was dissolved in 5 mL methanol. To it were added compound 1.4 (12 mg, 0.06 mmol), DBU (18 μL, 0.12 mmol) and CuI (15 mg, 0.08 mmol). The mixture was stirred at RT overnight, filtered and subjected to reverse-phase preparative HPLC to isolate the title compound 4.4. MS found for C₂₀H₁₆ClN₅O₃S (M+H)+ 442.1, 444.1 (Cl pattern).

### Example 57

### 5-Chloro-N-((1-(4-(2-oxopyridin-1(2H)-yl)-2-(piperidin-4-yloxy)phenyl)-1H-1,2,3-triazol-4-yl)methyl)thiophene-2-carboxamide (57)

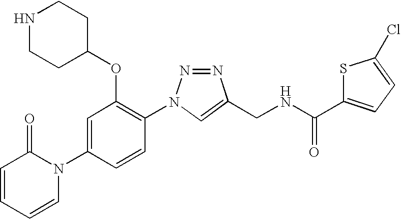

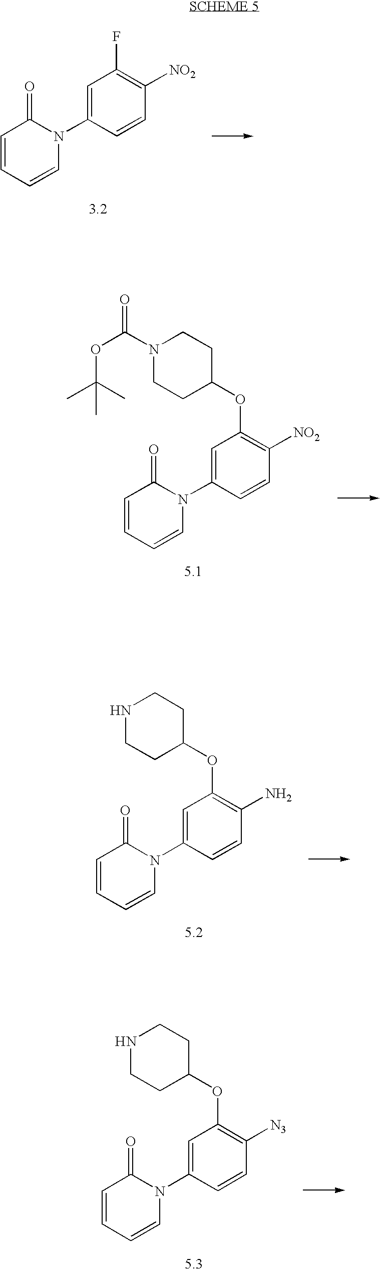

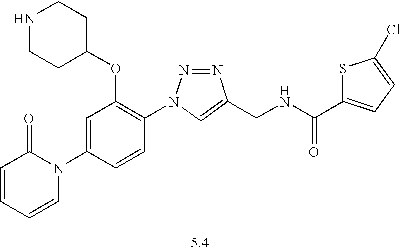

Step 1:

N—BOC-4-hydroxypiperidine (3.00 g, 14.8 mmol) was dissolved in 30 mL anhydrous DMSO. To it was added sodium hydride (60% in mineral oil, 0.60 g, 14.8 mmol) in small portions. The mixture was stirred at RT for 30 min. Compound 3.2 (1.74 g, 7.4 mmol) was dissolved in 20 mL anhydrous DMSO and was carefully added into the reaction mixture. The mixture was then stirred at 80° C. for 90 min. It was diluted with chloroform and washed with brine three times. The organic phase was dried, concentrated in vacuo and purified using flash column to give compound 5.1 (1.04 g, 34%). MS found for C₂₁H₂₅N₃O₆ (M+H)+ 416.1.

Step 2:

Compound 5.1 (1.00 g, 2.4 mmol) was dissolved in 100 mL ethanol and 50 mL acetic acid. It was treated with iron powder (0.81 g, 14.4 mmol) at 100° C. for 90 min and then diluted with acetonitrile and filtered through a celite bed. The filtrate was concentrated in vacuo and the residue was treated with 4N HCl in dioxane for 2 hrs. The mixture was concentrated in vauo and treated with 200 mL 1N NaOH. It was extracted with chloroform twice. The organic extracts were combined, washed with brine, dried and concentrated in vacuo to give compound 5.2 (0.51 g, 74%). MS found for C₁₆H₁₉N₃O₂ (M+H)+ 286.1.

Step 3:

To a solution of compound 5.2 (0.51 g, 1.8 mmol) in 10 mL TFA in ice bath was added sodium nitrite (125 mg, 1.8 mmol) in small portions. The mixture was stirred in ice bath for 30 min. To it was added an ice-cold solution of sodium azide (180 mg, 2.7 mmol) in 2 mL water. The mixture was stirred in ice bath for 3 hrs and directly subjected to reverse phase preparative HPLC to isolate compound 5.3 (67%). MS found for C₁₆H₁₇N₅O₂ (M+H)+ 312.1.

Step 4:

Compound 5.3 (180 mg, 0.42 mmol) was dissolved in 20 mL methanol. To it were added compound 1.4 (100 mg, 0.50 mmol), DBU (190 μL, 1.3 mmol) and CuI (160 mg, 0.84 mmol). The mixture was stirred at RT overnight. It was diluted with acetonitrile, filtered through a celite bed, concentrated in vacuo and subjected to reverse phase preparative HPLC to isolate the title compound 5.4. MS found for C₂₄H₂₃ClN₆O₃S (M+H)+ 511.0, 513.0 (Cl pattern).

### Example 58

### N-((1-(2-(1-Acetylpiperidin-4-yloxy)-4-(2-oxopyridin-1(2H)-yl)phenyl)-1H-1,2,3-triazol-4-yl)methyl)-5-chlorothiophene-2-carboxamide (58)

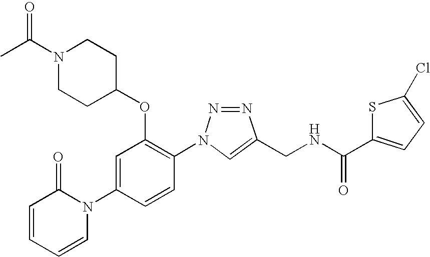

The title compound was prepared from 5-chloro-N-((1-(4-(2-oxopyridin-1(2H)-yl)-2-(piperidin-4-yloxy)phenyl)-1H-1,2,3-triazol-4-yl)methyl)thiophene-2-carboxamide using a similar procedure as described for Example 13. MS found for C₂₆H₂₅ClN₆O₄S (M+H)+ 553.1, 555.1 (Cl pattern).

### Example 59

### 4-(2-(4-((2-Chlorothiophene-5-carboxamido)methyl)-1H-1,2,3-triazol-1-yl)-5-(2-oxopyridin-1(2H)-yl)phenoxy)piperidine-1-carboxamide (59)

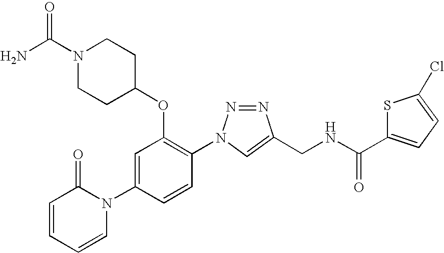

The title compound was prepared from 5-chloro-N-((1-(4-(2-oxopyridin-1(2H)-yl)-2-(piperidin-4-yloxy)phenyl)-1H-1,2,3-triazol-4-yl)methyl)thiophene-2-carboxamide using the same procedure described for Example 15. MS found for C₂₅H₂₄ClN₇O₄S (M+H)+ 554.1, 556.1 (Cl pattern).

### Example 60

### 5-Chloro-N-((1-(2-(1-methylpiperidin-4-yloxy)-4-(2-oxopyridin-1(2H)-yl)phenyl)-1H-1,2,3-triazol-4-yl)methyl)thiophene-2-carboxamide (60)

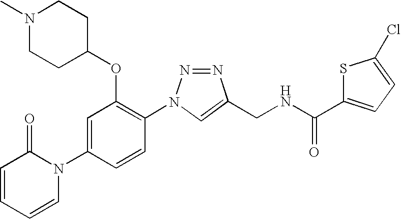

The title compound was prepared using a similar procedure as described for Example 57. MS found for C₂₅H₂₅ClN₆O₃S (M+H)+ 525.1, 527.1 (Cl pattern).

### Example 61

### 5-Chloro-N-((1-(2-(1-isopropylpiperidin-4-yloxy)-4-(2-oxopyridin-1(2H)-yl)phenyl)-1H-1,2,3-triazol-4-yl)methyl)thiophene-2-carboxamide (61)

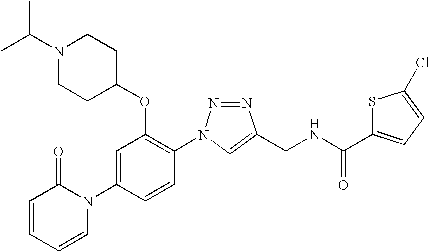

The title compound was prepared using a similar procedure as described for Example 57. MS found for C₂₇H₂₉ClN₆O₃S (M+H)+ 553.1, 555.1 (Cl pattern).

### Example 62

### 5-Chloro-N-((1-(2-(2-oxopiperidin-4-yloxy)-4-(2-oxopyridin-1(2H)-yl)phenyl)-1H-1,2,3-triazol-4-yl)methyl)thiophene-2-carboxamide (62)

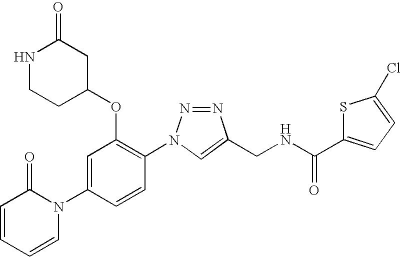

The title compound was prepared using a similar procedure as described for Example 57. MS found for C₂₄H₂₁ClN₆O₄S (M+H)+ 525.1, 527.1 (Cl pattern).

### Example 63

### 5-Chloro-N-((1-(4-(2-oxopyridin-1(2H)-yl)-2-(pyridin-4-yloxy)phenyl)-1H-1,2,3-triazol-4-yl)methyl)thiophene-2-carboxamide (63)

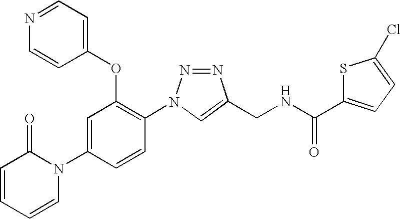

The title compound was prepared using a similar procedure as described for Example 57. MS found for C₂₄H₁₇ClN₆O₃S (M+H)+ 505.1, 507.1 (Cl pattern).

### Example 64

### 5-Chloro-N-((1-(4-(2-oxopyridin-1(2H)-yl)-2-(pyridin-3-yloxy)phenyl)-1H-1,2,3-triazol-4-yl)methyl)thiophene-2-carboxamide (64)

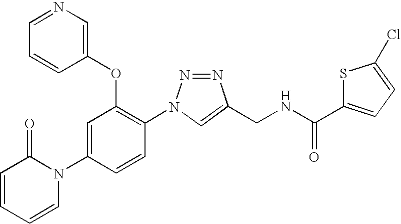

The title compound was prepared using a similar procedure as described for Example 57. MS found for C₂₄H₁₇ClN₆O₃S (M+H)+ 505.1, 507.1 (Cl pattern).

### Example 65

### 5-Chloro-N-((1-(4-(2-oxopyridin-1(2H)-yl)-2-(tetrahydro-2H-thiopyran-4-yloxy)phenyl)-1H-1,2,3-triazol-4-yl)methyl)thiophene-2-carboxamide (65)

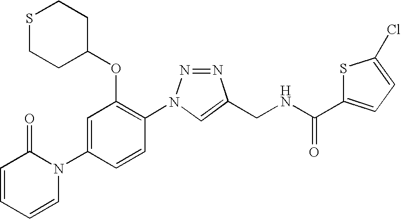

The title compound was prepared using a similar procedure as described for Example 57. MS found for C₂₄H₂₂ClN₅O₃S₂ (M+H)+ 528.1, 530.1 (Cl pattern).

### Example 66

### 5-Chloro-N-((1-(4-(2-oxopyridin-1(2H)-yl)-2-(1,1-dioxo-tetrahydro-2H-thiopyran-4-yloxy)phenyl)-1H-1,2,3-triazol-4-yl)methyl)thiophene-2-carboxamide (66)

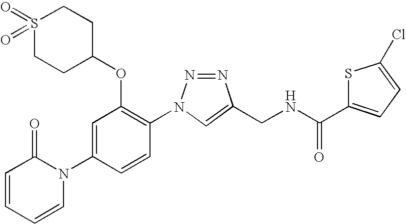

The title compound was prepared from 5-chloro-N-((1-(4-(2-oxopyridin-1(2H)-yl)-2-(tetrahydro-2H-thiopyran-4-yloxy)phenyl)-1H-1,2,3-triazol-4-yl)methyl)thiophene-2-carboxamide using a similar procedure as described for Example 12. MS found for C₂₄H₂₂ClN₅O₅S₂ (M+H)+ 560.1, 562.1 (Cl pattern).

### Example 67

### 5-Chloro-N-((1-(2-(3-(1,3-dioxoisoindolin-2-yl)propoxy)-4-(2-oxopyridin-1(2H)-yl)phenyl)-1H-1,2,3-triazol-4-yl)methyl)thiophene-2-carboxamide (67)

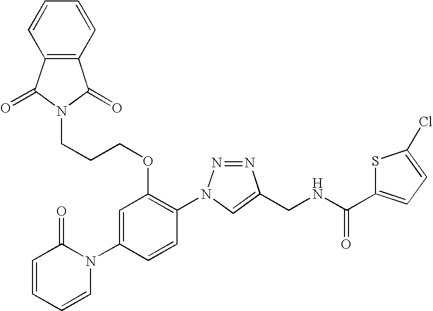

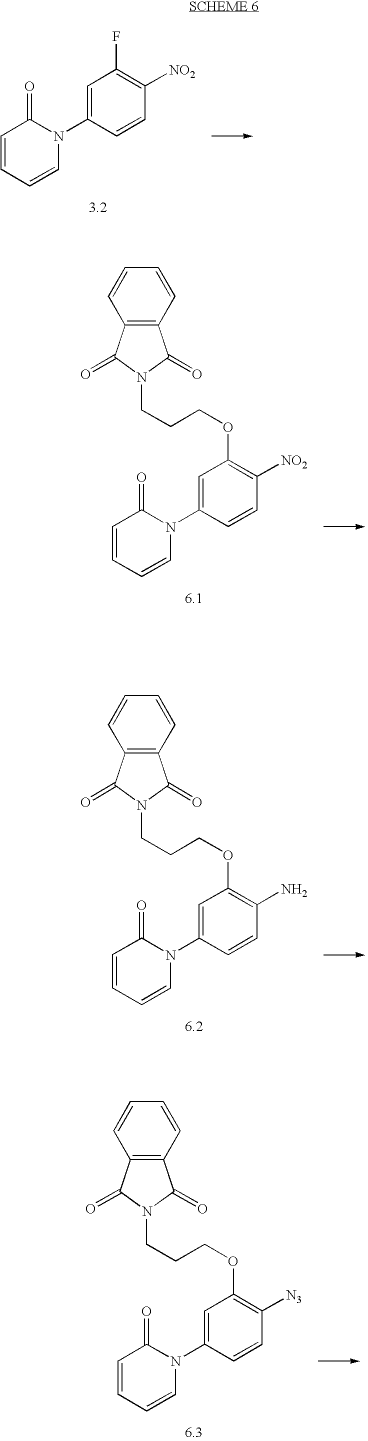

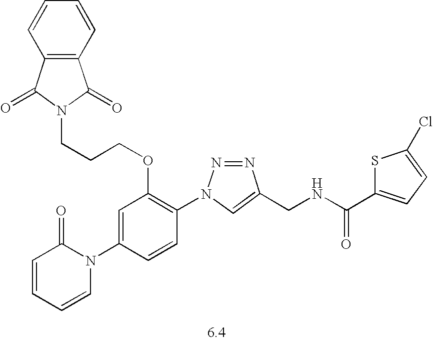

Step 1:

N-(3-Hydroxypropyl)phthalimide (1.68 g, 8.2 mmol) was dissolved in 20 mL anhydrous DMSO. To it was added sodium hydride (60% in mineral oil, 0.33 g, 8.2 mmol). After stirring for 1 hr at RT, a solution of compound 3.2 (0.96 g, 4.1 mmol) in 10 mL anhydrous DMSO was added. The mixture was stirred for 1 hr in 80° C. bath. It was diluted with chloroform and washed with brine three times. The organic phase was dried, concentrated and purified using flash column to afford compound 6.1 (45%). MS found for C₂₂H₁₇N₃O₆ (M+H)+ 420.1.

Step 2:

Compound 6.1 (700 mg, 1.6 mmol) was dissolved in 30 mL ethanol and 15 mL acetic acid. It was treated with iron powder (450 mg, 8.0 mmol) in 100° C. bath for 90 min. It was diluted with acetonitrile, filtered through a celite bed, concentrated and purified using reverse phase HPLC to isolate compound 6.2 (57%). MS found for C₂₂H₁₉N₃O₄ (M+H)+ 390.1.

Step 3:

Compound 6.2 (200 mg, 0.51 mmol) was dissolved in 7 mL TFA and stirred in ice bath. To it was added sodium nitrite (36 mg, 0.51 mmol). The mixture was stirred in ice bath for 30 min. Then an ice-cold solution of sodium azide (50 mg, 0.77 mmol) in 1 mL water was added. The mixture was stirred for 2 hrs in ice bath, diluted with 300 mL ethyl acetate, washed with brine twice and sodium bicarbonate saturated aq. solution once. The organic phase was dried and concentrated in vacuo to give compound 6.3 in quantitative yield. MS found for C₂₂H₁₇N₅O₄ (M+H)+ 416.1.

Step 4:

Compound 6.3 (0.51 mmol) was dissolved in 10 mL methanol. To it were added compound 1.4 (101 mg, 0.51 mmol), DBU (160 μL, 1.02 mmol) and CuI (200 mg, 1.02 mmol). The mixture was stirred at RT overnight, diluted with acetonitrile, filtered through a celite bed, concentrated in vacuo and subjected to direct reverse phase preparative HPLC to isolate title compound 6.4. MS found for C₃₀H₂₃ClN₆O₅S (M+H)+ 615.1, 617.1 (Cl pattern).

### Example 68

### N-((1-(2-(3-Aminopropoxy)-4-(2-oxopyridin-1(2H)-yl)phenyl)-1H-1,2,3-triazol-4-yl)methyl)-5-chlorothiophene-2-carboxamide (68)

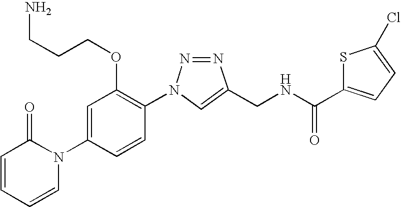

To a solution of compound 6.4 (16 mg, 0.026 mmol) in 10 mL methanol was added hydrazine monohydrate (15 μL, 0.30 mmol). The mixture was stirred at 80° C. for 2 hrs and then directly subjected to reverse phase preparative HPLC to isolate the title compound. MS found for C₂₂H₂₁ClN₆O₃S (M+H)+ 485.1, 487.1 (Cl pattern).

### Example 69

### N-((1-(2-(3-Acetamidopropoxy)-4-(2-oxopyridin-1(2H)-yl)phenyl)-1H-1,2,3-triazol-4-yl)methyl)-5-chlorothiophene-2-carboxamide (69)

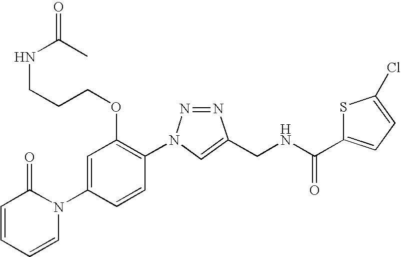

The title compound was prepared from N-((1-(2-(3-aminopropoxy)-4-(2-oxopyridin-1(2H)-yl)phenyl)-1H-1,2,3-triazol-4-yl)methyl)-5-chlorothiophene-2-carboxamide (as shown in Example 68) using a similar procedure as described in Example 13. MS found for C₂₄H₂₃ClN₆O₄S (M+H)+ 527.1, 529.1 (Cl pattern).

### Example 70

### 1-(3-(2-(4-((2-Chlorothiophene-5-carboxamido)methyl)-1H-1,2,3-triazol-1-yl)-5-(2-oxopyridin-1(2H)-yl)phenoxy)propyl)urea (70)

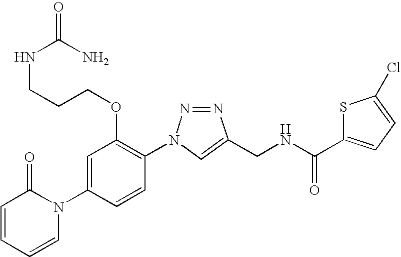

The title compound was prepared from N-((1-(2-(3-aminopropoxy)-4-(2-oxopyridin-1(2H)-yl)phenyl)-1H-1,2,3-triazol-4-yl)methyl)-5-chlorothiophene-2-carboxamide using a similar procedure as described in Example 15. MS found for C₂₃H₂₂ClN₇O₄S (M+H)+ 528.1, 530.1 (Cl pattern).

### Example 71

### N-((1-(2-(2-Aminoethoxy)-4-(2-oxopyridin-1(2H)-yl)phenyl)-1H-1,2,3-triazol-4-yl)methyl)-5-chlorothiophene-2-carboxamide (71)

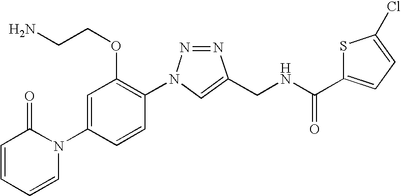

The title compound was prepared using a similar procedure as described in Example 68. MS found for C₂₁H₁₉ClN₆O₃S (M+H)+ 471.1, 473.1 (Cl pattern).

### Example 72

### N-((1-(2-(2-Acetamidoethoxy)-4-(2-oxopyridin-1(2H)-yl)phenyl)-1H-1,2,3-triazol-4-yl)methyl)-5-chlorothiophene-2-carboxamide (72)

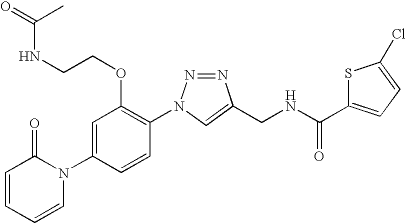

The title compound was prepared using a similar procedure as described in Example 69. MS found for C₂₃H₂₁ClN₆O₄S (M+H)+ 513.1, 515.1 (Cl pattern).

### Example 73

### 1-(2-(2-(4-((2-Chlorothiophene-5-carboxamido)methyl)-1H-1,2,3-triazol-1-yl)-5-(2-oxopyridin-1(2H)-yl)phenoxy)ethyl)urea (73)

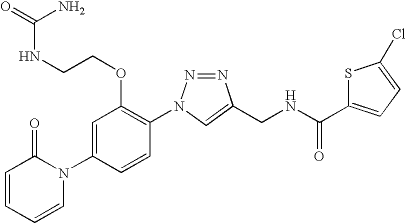

The title compound was prepared using a similar procedure as described in Example 70. MS found for C₂₂H₂₀ClN₇O₄S (M+H)+ 514.1, 516.1 (Cl pattern).

### Example 74

### 5-Chloro-N-((1-(2-(3-hydroxypropoxy)-4-(2-oxopyridin-1(2H)-yl)phenyl)-1H-1,2,3-triazol-4-yl)methyl)thiophene-2-carboxamide (74)

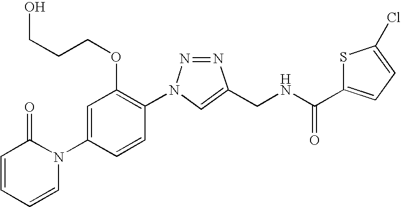

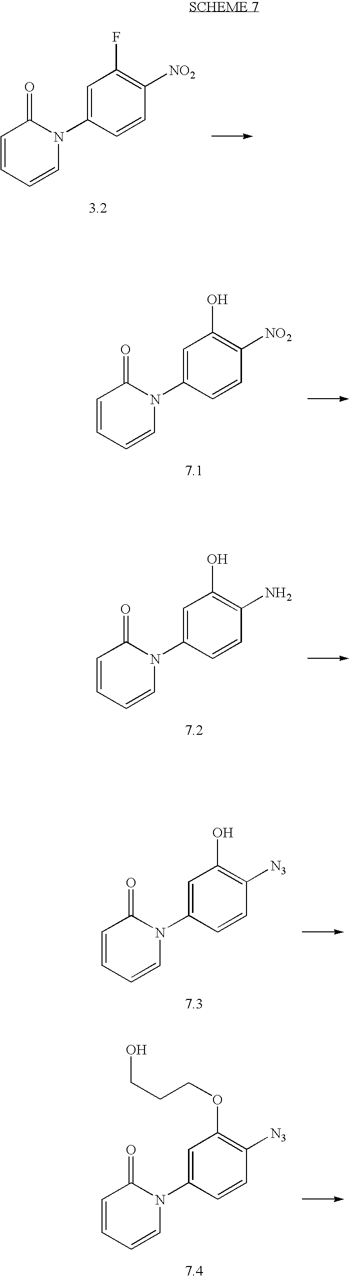

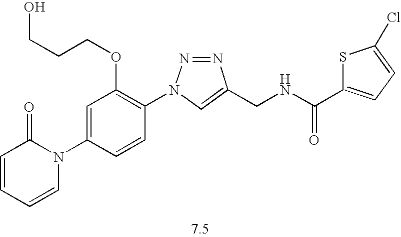

Step 1:

Compound 3.2 (1.00 g, 4.27 mmol) was dissolved in 10 mL dioxane and 10 mL water. It was treated with sodium hydroxide (0.34 g, 8.6 mmol) at 60° C. for 2 hr. It was acidified using 2N HCl till pH=1. It was extracted with ethyl acetate twice. The organic extracts were combined, dried and concentrated in vacuo to afford compound 7.1 in 96% yield (0.95 g). MS found for C₁₁H₈N₂O₄ (M+H)+ 233.0.

Step 2:

Compound 7.1 (0.95 g, 4.1 mmol) was stirred in 60 mL ethanol and 20 mL water. To it were added ammonium chloride (2.2 g, 41 mmol) and indium powder (1.9 g, 16.4 mmol). The mixture was heated in 100° C. bath for 6 hrs. It was concentrated in vacuo to remove ethanol. The residue was titrated with saturated sodium carbonate aq. solution till pH=7.5. The mixture was extracted with CHCl₃/iPrOH (3:1) four times. The organic extracts were combined, dried and concentrated in vacuo to give compound 7.2 in 80% yield (0.66 g). MS found for C₁₁H₁₀N₂O₂ (M+H)+ 203.1.

Step 3:

Compound 7.2 (0.66 g, 3.2 mmol) was dissolved in 50 mL concentrated HCl and stirred in ice bath. To it was added an ice-cold solution of sodium nitrite (0.33 g, 4.8 mmol) in 4 mL water dropwise. The mixture was stirred for 40 min. Then an ice-cold solution of sodium azide (0.62 g, 9.6 mmol) in 5 mL water was added. The mixture was stirred overnight. The mixture was then extracted with ethyl acetate three times. The combined organic extracts were washed with brine, dried and concentrated in vacuo to give compound 7.3 (0.63 g, 86%). MS found for C₁₁H₈N₄O₂ (M+H)+ 229.1.

Step 4:

Compound 7.3 (55 mg, 0.24 mmol) was dissolved in 3 mL DMF. To it were added K₂CO₃ (132 mg, 0.96 mmol) followed by 3-bromo-1-propanol (78 μL, 0.96 mmol). The mixture was stirred overnight at RT and then directly subjected to flash column to isolate compound 7.4 (39 mg, 57%). MS found for C₁₄H₁₄N₄O₃ (M+H)+ 287.1.

Step 5:

Compound 7.4 (39 mg, 0.14 mmol) was dissolved in 5 mL methanol. To it were added compound 1.4 (28 mg, 0.14 mmol), DBU (42 μL, 0.28 mmol) and CuI (53 mg, 0.28 mmol). The mixture was stirred overnight, diluted with acetonitrile, filtered through a celite bed, concentrated and subjected to reverse phase preparative HPLC to isolate the title compound 7.5. MS found for C₂₂H₂₀ClN₅O₄S (M+H)+ 486.1, 488.1 (Cl pattern).

### Example 75

### 5-Chloro-N-((1-(2-(2-hydroxyethoxy)-4-(2-oxopyridin-1(2H)-yl)phenyl)-1H-1,2,3-triazol-4-yl)methyl)thiophene-2-carboxamide (75)

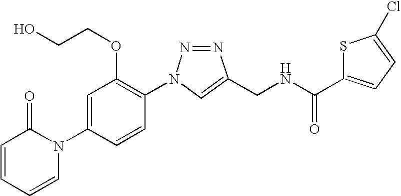

The title compound was prepared using a procedure similar to that described in Example 74. MS found for C₂₁H₁₈ClN₅O₄S (M+H)+ 472.1, 474.1 (Cl pattern).

### Example 76

### 5-Chloro-N-((1-(2-(2-methoxyethoxy)-4-(2-oxopyridin-1(2H)-yl)phenyl)-1H-1,2,3-triazol-4-yl)methyl)thiophene-2-carboxamide (76)

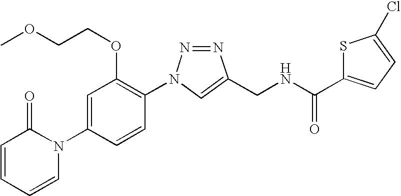

The title compound was prepared using a procedure similar to that described in Example 74. MS found for C₂₂H₂₀ClN₅O₄S (M+H)+ 486.1, 488.1 (Cl pattern).

### Example 77

### 5-Chloro-N-((1-(2-(3-methoxypropoxy)-4-(2-oxopyridin-1(2H)-yl)phenyl)-1H-1,2,3-triazol-4-yl)methyl)thiophene-2-carboxamide (77)

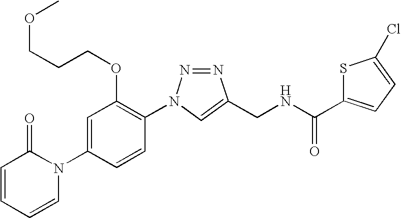

The title compound was prepared using a procedure similar to that described in Example 74. MS found for C₂₃H₂₂ClN₅O₄S (M+H)+ 500.1, 502.1 (Cl pattern).

### Example 78

### 5-Chloro-N-((1-(2-((R)-2,3-dihydroxypropoxy)-4-(2-oxopyridin-1(2H)-yl)phenyl)-1H-1,2,3-triazol-4-yl)methyl)thiophene-2-carboxamide (78)

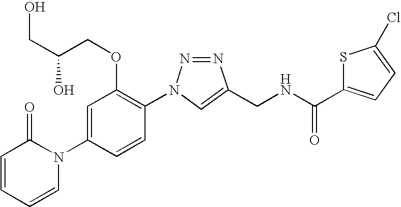

The title compound was prepared using a procedure similar to that described in Example 74. MS found for C₂₂H₂₀ClN₅O₅S (M+H)+ 502.1, 504.1 (Cl pattern).

### Example 79

### 5-Chloro-N-((1-(2-((S)-2,3-dihydroxypropoxy)-4-(2-oxopyridin-1(2H)-yl)phenyl)-1H-1,2,3-triazol-4-yl)methyl)thiophene-2-carboxamide (79)

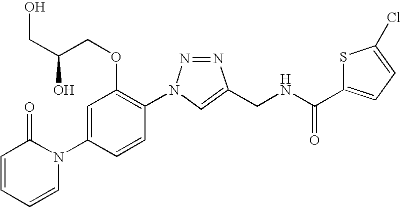

The title compound was prepared using a procedure similar to that described in Example 74. MS found for C₂₂H₂₀ClN₅O₅S (M+H)+ 502.1, 504.1 (Cl pattern).

### Example 80

### 5-Chloro-N-((1-(2-(2-(methylsulfonyl)ethoxy)-4-(2-oxopyridin-1(2H)-yl)phenyl)-1H-1,2,3-triazol-4-yl)methyl)thiophene-2-carboxamide (80)

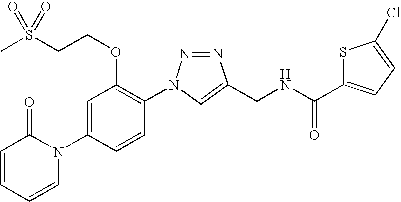

The title compound was prepared using a procedure similar to that described in Example 74. MS found for C₂₂H₂₀ClN₅O₅S₂ (M+H)+ 534.1, 536.1 (Cl pattern).

### Example 81

### 5-Chloro-N-((1-(2-(2-(aminosulfonyl)ethoxy)-4-(2-oxopyridin-1(2H)-yl)phenyl)-1H-1,2,3-triazol-4-yl)methyl)thiophene-2-carboxamide (81)

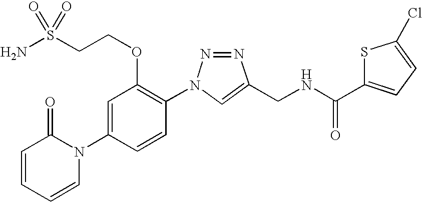

The title compound was prepared using a procedure similar to that described in Example 74. MS found for C₂₁H₁₉ClN₆O₅S₂ (M+H)+ 535.1, 537.1 (Cl pattern).

### Example 82

### 5-Chloro-N-((1-(2-(2-(ethylsulfonyl)ethoxy)-4-(2-oxopyridin-1(2H)-yl)phenyl)-1H-1,2,3-triazol-4-yl)methyl)thiophene-2-carboxamide (82)

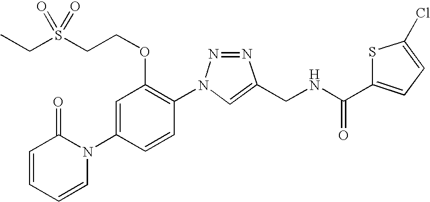

The title compound was prepared using a procedure similar to that described in Example 74. MS found for C₂₃H₂₂ClN₅O₅S₂ (M+H)+ 548.1, 550.1 (Cl pattern).

### Example 83

### 5-Chloro-N-((1-(2-(3-(methylsulfonyl)propoxy)-4-(2-oxopyridin-1(2H)-yl)phenyl)-1H-1,2,3-triazol-4-yl)methyl)thiophene-2-carboxamide (83)

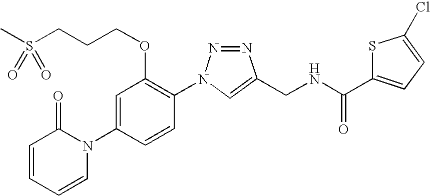

The title compound was prepared using a procedure similar to that described in Example 74. MS found for C₂₃H₂₂ClN₅O₅S₂ (M+H)+ 548.1, 550.1 (Cl pattern).

### Example 84

### 5-Chloro-N-((1-(2-(3-(aminosulfonyl)propoxy)-4-(2-oxopyridin-1(2H)-yl)phenyl)-1H-1,2,3-triazol-4-yl)methyl)thiophene-2-carboxamide (84)

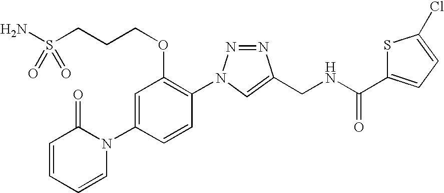

The title compound was prepared using a procedure similar to that described in Example 74. MS found for C₂₂H₂₁ClN₆O₅S₂ (M+H)+ 549.1, 551.1 (Cl pattern).

### Example 85

### 5-Chloro-N-((1-(2-(2-(dimethylamino)ethoxy)-4-(2-oxopyridin-1(2H)-yl)phenyl)-1H-1,2,3-triazol-4-yl)methyl)thiophene-2-carboxamide (85)

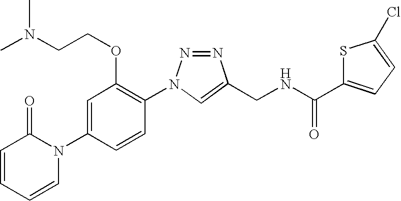

The title compound was prepared using a procedure similar to that described in Example 74. MS found for C₂₃H₂₃ClN₆O₃S (M+H)+ 499.1, 501.1 (Cl pattern).

### Example 86

### 5-Chloro-N-((1-(2-(2-(dimethyl(dimethylamino)amino)ethoxy)-4-(2-oxopyridin-1(2H)-yl)phenyl)-1H-1,2,3-triazol-4-yl)methyl)thiophene-2-carboxamide (86)

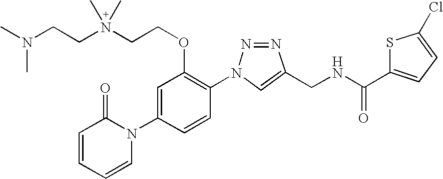

The title compound was prepared using a procedure similar to that described in Example 74. MS found for C₂₇H₃₃ClN₇O₃S M+ 570.1.

### Example 87

### 5-Chloro-N-((1-(2-(2-(methylamino)ethoxy)-4-(2-oxopyridin-1(2H)-yl)phenyl)-1H-1,2,3-triazol-4-yl)methyl)thiophene-2-carboxamide (87)

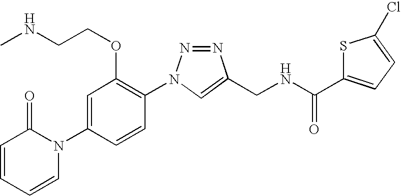

The title compound was prepared using a procedure similar to that described in Example 74. MS found for C₂₂H₂₁ClN₆O₃S (M+H)+ 485.1, 587.1 (Cl pattern).

### Example 88

### 5-Chloro-N-((1-(4-(2-oxopyridin-1(2H)-yl)-2-(2-(pyrrolidin-1-yl)ethoxy)phenyl)-1H-1,2,3-triazol-4-yl)methyl)thiophene-2-carboxamide (88)

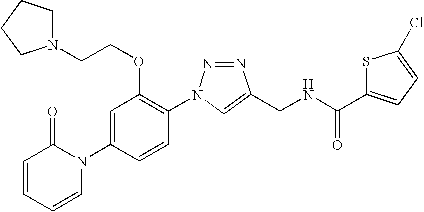

The title compound was prepared using a procedure similar to that described in Example 74. MS found for C₂₅H₂₅ClN₆O₃S (M+H)+ 525.1, 527.1 (Cl pattern).

### Example 89

### 5-Chloro-N-((1-(4-(2-oxopyridin-1(2H)-yl)-2-(2-(piperidin-1-yl)ethoxy)phenyl)-1H-1,2,3-triazol-4-yl)methyl)thiophene-2-carboxamide (89)

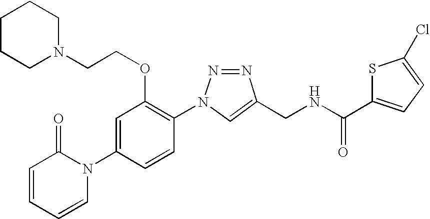

The title compound was prepared using a procedure similar to that described in Example 74. MS found for C₂₆H₂₇ClN₆O₃S (M+H)+ 539.1, 541.1 (Cl pattern).

### Example 90

### 5-Chloro-N-((1-(2-(2-morpholinoethoxy)-4-(2-oxopyridin-1(2H)-yl)phenyl)-1H-1,2,3-triazol-4-yl)methyl)thiophene-2-carboxamide (90)

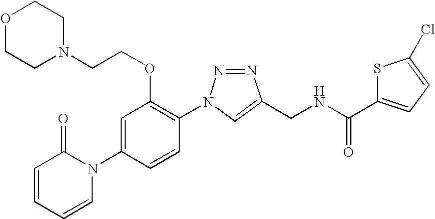

The title compound was prepared using a procedure similar to that described in Example 74. MS found for C₂₅H₂₅ClN₆O₄S (M+H)+ 541.1, 543.1 (Cl pattern).

### Example 91

### 5-Chloro-N-((1-(4-(2-oxopyridin-1(2H)-yl)-2-(2-(pyridin-4-yl)ethoxy)phenyl)-1H-1,2,3-triazol-4-yl)methyl)thiophene-2-carboxamide (91)

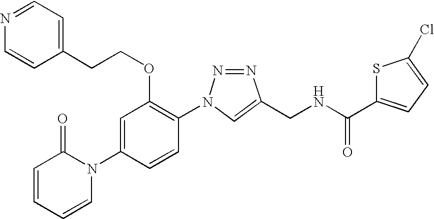

The title compound was prepared using a procedure similar to that described in Example 74. MS found for C₂₆H₂₁ClN₆O₃S (M+H)+ 533.1, 535.1 (Cl pattern).

### Example 92

### 5-Chloro-N-((1-(2-(3-(dimethylamino)propoxy)-4-(2-oxopyridin-1(2H)-yl)phenyl)-1H-1,2,3-triazol-4-yl)methyl)thiophene-2-carboxamide (92)

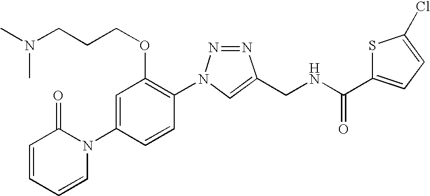

The title compound was prepared using a procedure similar to that described in Example 74. MS found for C₂₄H₂₅ClN₆O₃S (M+H)+ 513.1, 515.1 (Cl pattern).

### Example 93

### 5-Chloro-N-((1-(4-(2-oxopyridin-1(2H)-yl)-2-(3-(pyrrolidin-1-yl)propoxy)phenyl)-1H-1,2,3-triazol-4-yl)methyl)thiophene-2-carboxamide (93)

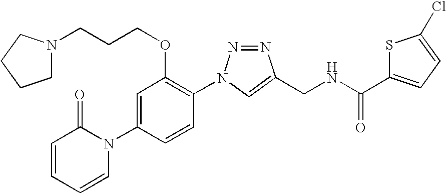

The title compound was prepared using a procedure similar to that described in Example 74. MS found for C₂₆H₂₇ClN₆O₃S (M+H)+ 539.1, 541.1 (Cl pattern).

### Example 94

### 5-Chloro-N-((1-(4-(2-oxopyridin-1(2H)-yl)-2-(3-(piperidin-1-yl)propoxy)phenyl)-1H-1,2,3-triazol-4-yl)methyl)thiophene-2-carboxamide (94)

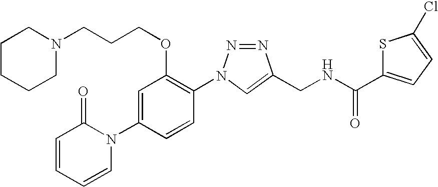

The title compound was prepared using a procedure similar to that described in Example 74. MS found for C₂₇H₂₉ClN₆O₃S (M+H)+ 553.1, 555.1 (Cl pattern).

### Example 95

### 5-Chloro-N-((1-(2-(3-morpholinopropoxy)-4-(2-oxopyridin-1(2H)-yl)phenyl)-1H-1,2,3-triazol-4-yl)methyl)thiophene-2-carboxamide (95)

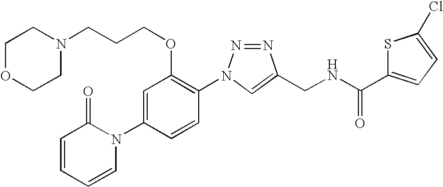

The title compound was prepared using a procedure similar to that described in Example 74. MS found for C₂₆H₂₇ClN₆O₄S (M+H)+ 555.1, 557.1 (Cl pattern).

### Example 96

### 2-(2-(4-((2-Chlorothiophene-5-carboxamido)methyl)-1H-1,2,3-triazol-1-yl)-5-(2-oxopyridin-1(2H)-yl)phenoxy)acetic acid (96)

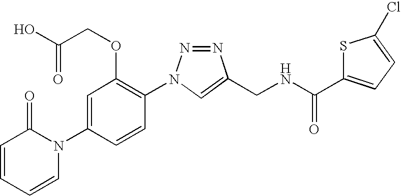

The title compound was prepared using a procedure similar to that described in Example 74. MS found for C₂₁H₁₆ClN₅O₅S (M+H)+ 486.1, 488.1 (Cl pattern).

### Example 97

### N-((1-(2-(2-Amino-2-oxoethoxy)-4-(2-oxopyridin-1(2H)-yl)phenyl)-1H-1,2,3-triazol-4-yl)methyl)-5-chlorothiophene-2-carboxamide (97)

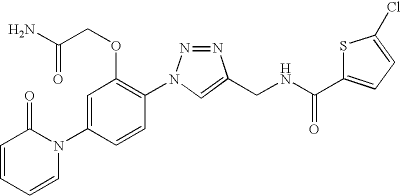

The title compound was prepared using a procedure similar to that described in Example 74. MS found for C₂₁H₁₇ClN₆O₄S (M+H)+ 485.1, 487.1 (Cl pattern).

### Example 98

### N-((1-(2-((1H-Tetrazol-5-yl)methoxy)-4-(2-oxopyridin-1(2H)-yl)phenyl)-1H-1,2,3-triazol-4-yl)methyl)-5-chlorothiophene-2-carboxamide (98)

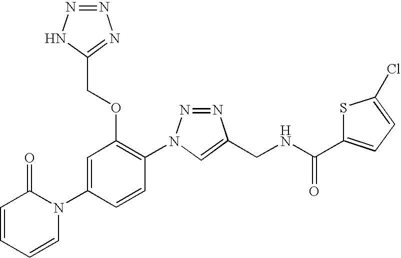

The title compound was prepared using a procedure similar to that described in Example 74. MS found for C₂₁H₁₆ClN₉O₃S (M+H)+ 510.1, 512.1 (Cl pattern).

### Example 99

### 5-Chloro-N-((1-(4-(2-oxopyridin-1(2H)-yl)-2-(trifluoromethoxy)phenyl)-1H-1,2,3-triazol-4-yl)methyl)thiophene-2-carboxamide (99)

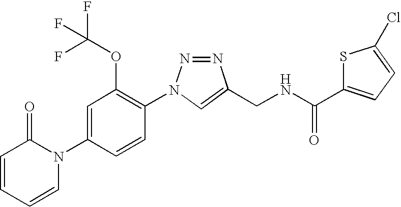

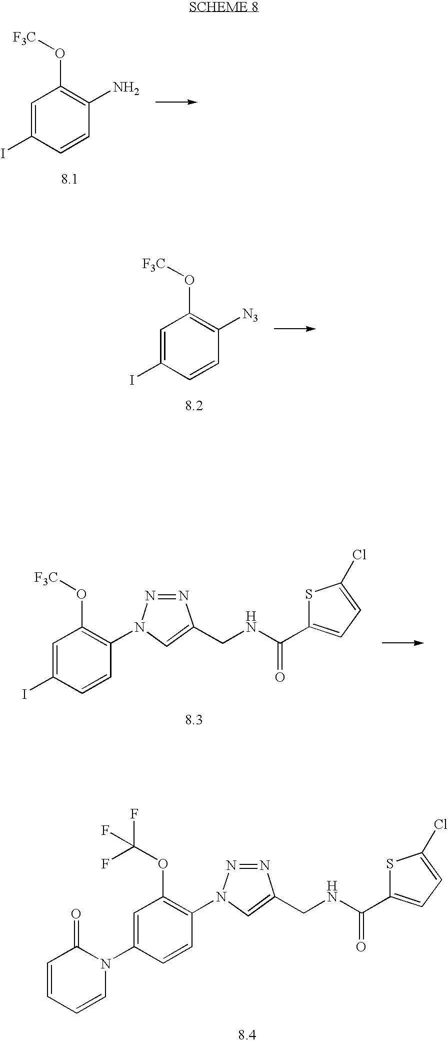

Step 1:

4-Iodo-2-trifluoromethoxyaniline (1.68 g, 5.5 mmol) was stirred in 40 mL conc. HCl in ice bath. To it was added an ice-cold solution of sodium nitrite (0.57 g, 8.3 mmol) in 2 mL water dropwise. The mixture was stirred at RT for 30 min. Then an ice-cold solution of sodium azide (1.08 g, 16.6 mmol) in 5 mL water was added. The mixture was stirred overnight before being extracted with ethyl acetate twice. The organic extracts were combined, washed with brine, dried, and concentrated in vacuo to afford compound 8.2 (1.69 g, 93%).

Step 2:

Compound 8.2 (1.69 g, 5.1 mmol) was dissolved in 50 mL methanol. To it were added compound 1.4 (1.54 g, 7.7 mmol), DBU (1.52 mL, 10.2 mmol) and CuI (1.94 g, 10.2 mmol). The mixture was stirred overnight, diluted with acetonitrile, filtered through celite bed, concentrated, purified using flash column to give compound 8.3 (1.85 g, 69%). MS found for C₁₅H₉ClF₃₁N₄O₂S (M+H)+ 529.0.

Step 3:

Compound 8.3 (180 mg, 0.34 mmol) was dissolved in 5 mL dioxane and 1 mL DMSO in a sealed tube. To it were added 2-hydroxypyridine (130 mg, 1.36 mmol), N,N′-dimethylethylenediamine (22 μL, 0.2 mmol), K₃PO₄ (144 mg, 0.68 mmol) and CuI (32 mg, 0.17 mmol). The mixture was stirred at 120° C. for 16 hrs, filtered, concentrated and subjected to reverse phase preparative HPLC to isolate the title compound 8.4. MS found for C₂₀H₁₃ClF₃N₅O₃S (M+H)+ 496.0, 498.0 (Cl pattern).

### Example 100

### 2-(4-((2-Chlorothiophene-5-carboxamido)methyl)-1H-1,2,3-triazol-1-yl)-5-(2-oxopyridin-1(2H)-yl)benzoic acid (100)

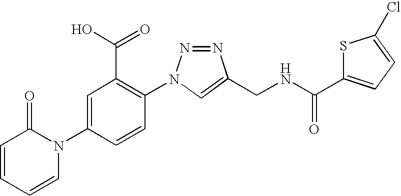

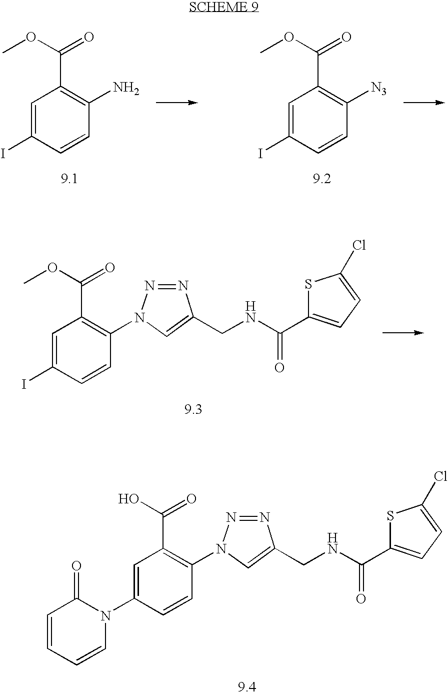

Step 1:

Methyl 2-amino-5-iodonezoate (3.00 g, 10.8 mmol) was stirred in 35 mL TFA in ice bath. To it was added sodium nitrite (820 mg, 12 mmol) in small portions. The mixture was stirred in ice bath for 40 min. To it was added an ice-cold solution of sodium azide (1.41 g, 21.6 mmol) in 8 mL water. The mixture was stirred for 3 hrs, diluted with ethyl acetate (500 mL), washed with brine three times, dried, and concentrated in vacuo to give compound 9.2 in quantitative yield.

Step 2:

Compound 9.2 (10.8 mmol) was dissolved in 200 mL methanol. To it were added compound 1.4 (2.58 g, 13.0 mmol), DBU (4.8 mL, 32 mmol) and CuI (4.1 g, 21 mmol). The mixture was stirred overnight, diluted with 600 mL ethyl acetate, filtered through a celite bed. The filtrate was washed with saturated ammonium chloride aq. solution and brine, dried, and concentrated in vacuo to give compound 9.3 in quantitative yield. MS found for C₁₆H₁₂ClIN₄O₃S (M+H)+ 503.0, 505.0 (Cl pattern).

Step 3:

Compound 9.3 (2.00 g, 4.0 mmol) was dissolved in 50 mL DMSO. To it were added 2-hydroxypyridine (0.76 g, 8.0 mmol), 8-hydroxyquinoline (174 mg, 1.2 mmol), cesium carbonate (2.61 g, 8.0 mmol) and CuI (230 mg, 1.2 mmol). The mixture was stirred at 120° C. for 18 hrs. It was then filtered and directly subjected to reverse phase preparative HPLC to isolate the title compound 9.4. MS found for C₂₀H₁₄ClN₅O₄S (M+H)+ 456.0, 458.0 (Cl pattern).

### Example 101

### N-((1-(2-Carbamoyl-4-(2-oxopyridin-1(2H)-yl)phenyl)-1H-1,2,3-triazol-4-yl)methyl)-5-chlorothiophene-2-carboxamide (101)

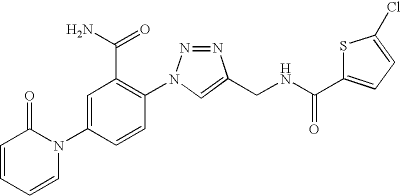

2-(4-((2-Chlorothiophene-5-carboxamido)methyl)-1H-1,2,3-triazol-1-yl)-5-(2-oxopyridin-1(2H)-yl)benzoic acid (13 mg, 0.03 mmol) was dissolved in 2 mL DMF. To it were added ammonia (0.5M in dioxane, 180 μL, 0.09 mmol), DIEA (26 μL, 0.15 mmol) and PyBOP (47 mg, 0.09 mmol) in order. The mixture was stirred at RT for 17 hrs and directly subjected to reverse phase preparative HPLC to isolate the title compound. MS found for C₂₀H₁₅ClN₆O₃S (M+H)+ 455.1, 457.1 (Cl pattern).

### Example 102

### 5-Chloro-N-((1-(2-(methylcarbamoyl)-4-(2-oxopyridin-1(2H)-yl)phenyl)-1H-1,2,3-triazol-4-yl)methyl)thiophene-2-carboxamide (102)

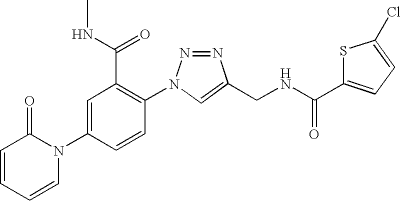

The title compound was prepared using a similar procedure as described in Example 101. MS found for C₂₁H₁₇ClN₆O₃S (M+H)+ 469.1, 471.1 (Cl pattern).

### Example 103

### 5-Chloro-N-((1-(2-(dimethylcarbamoyl)-4-(2-oxopyridin-1(2H)-yl)phenyl)-1H-1,2,3-triazol-4-yl)methyl)thiophene-2-carboxamide (103)

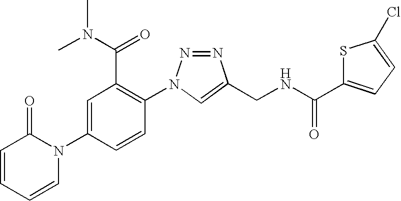

The title compound was prepared using a similar procedure as described in Example 101. MS found for C₂₂H₁₉ClN₆O₃S (M+H)+ 483.1, 485.1 (Cl pattern).

### Example 104

### N-((1-(2-((2-Hydroxyethyl)carbamoyl)-4-(2-oxopyridin-1(2H)-yl)phenyl)-1H-1,2,3-triazol-4-yl)methyl)-5-chlorothiophene-2-carboxamide (104)

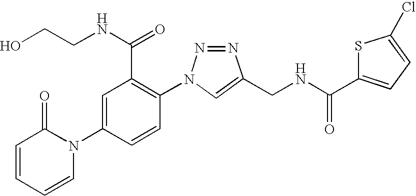

The title compound was prepared using a similar procedure as described in Example 101. MS found for C₂₂H₁₉ClN₆O₄S (M+H)+ 499.1, 501.1 (Cl pattern).

### Example 105

### N-((1-(2-((3-Hydroxypropyl)carbamoyl)-4-(2-oxopyridin-1(2H)-yl)phenyl)-1H-1,2,3-triazol-4-yl)methyl)-5-chlorothiophene-2-carboxamide (105)

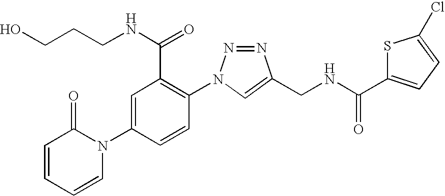

The title compound was prepared using a similar procedure as described in Example 101. MS found for C₂₃H₂₁ClN₆O₄S (M+H)+ 513.1, 515.1 (Cl pattern).

### Example 106

### N-((1-(2-((2-Methoxyethyl)carbamoyl)-4-(2-oxopyridin-1(2H)-yl)phenyl)-1H-1,2,3-triazol-4-yl)methyl)-5-chlorothiophene-2-carboxamide (106)

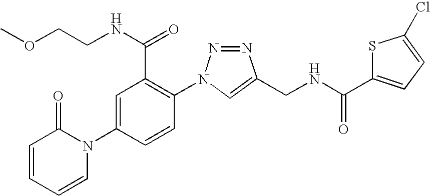

The title compound was prepared using a similar procedure as described in Example 101. MS found for C₂₃H₂₁ClN₆O₄S (M+H)+ 513.1, 515.1 (Cl pattern).

### Example 107

### N-((1-(2-((2-Aminoethyl)carbamoyl)-4-(2-oxopyridin-1(2H)-yl)phenyl)-1H-1,2,3-triazol-4-yl)methyl)-5-chlorothiophene-2-carboxamide (107)

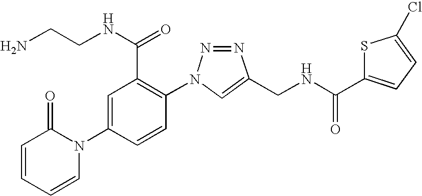

The title compound was prepared using a similar procedure as described in Example 101. MS found for C₂₂H₂₀ClN₇O₃S (M+H)+ 498.1, 500.1 (Cl pattern).

### Example 108

### N-((1-(2-((2-Amino-2-oxoethyl)carbamoyl)-4-(2-oxopyridin-1(2H)-yl)phenyl)-1H-1,2,3-triazol-4-yl)methyl)-5-chlorothiophene-2-carboxamide (108)

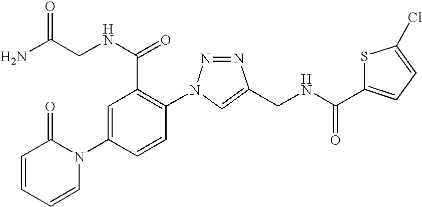

The title compound was prepared using a similar procedure as described in Example 101. MS found for C₂₂H₁₈ClN₇O₄S (M+H)+ 512.1, 514.1 (Cl pattern).

### Example 109

### N-((1-(2-((2-(Dimethylamino)ethyl)carbamoyl)-4-(2-oxopyridin-1(2H)-yl)phenyl)-1H-1,2,3-triazol-4-yl)methyl)-5-chlorothiophene-2-carboxamide (109)

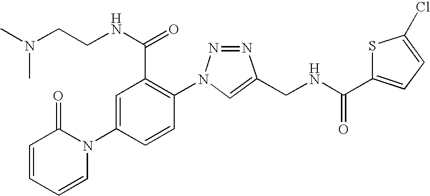

The title compound was prepared using a similar procedure as described in Example 101. MS found for C₂₄H₂₄ClN₇O₃S (M+H)+ 526.1, 528.1 (Cl pattern).

### Example 110

### 5-Chloro-N-((1-(2-(methyl(2-(methylamino)ethyl)carbamoyl)-4-(2-oxopyridin-1(2H)-yl)phenyl)-1H-1,2,3-triazol-4-yl)methyl)thiophene-2-carboxamide (110)

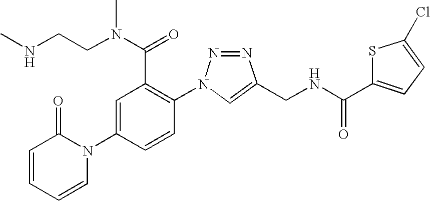

The title compound was prepared using a similar procedure as described in Example 101. MS found for C₂₄H₂₄ClN₇O₃S (M+H)+ 526.1.

### Example 111

### 5-Chloro-N-((1-(2-((2-(dimethylamino)ethyl)(methyl)carbamoyl)-4-(2-oxopyridin-1(2H)-yl)phenyl)-1H-1,2,3-triazol-4-yl)methyl)thiophene-2-carboxamide (111)

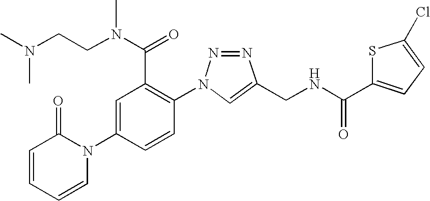

The title compound was prepared using a similar procedure as described in Example 101. MS found for C₂₅H₂₆ClN₇O₃S (M+H)+ 540.1.

### Example 112

### N-((1-(2-(3-aminopropylcarbamoyl)-4-(2-oxopyridin-1(2H)-yl)phenyl)-1H-1,2,3-triazol-4-yl)methyl)-5-chlorothiophene-2-carboxamide (112)

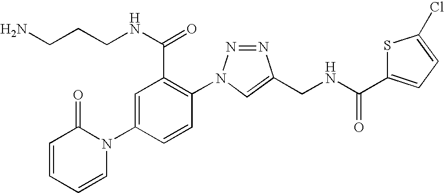

The title compound was prepared using a similar procedure as described in Example 101. MS found for C₂₃H₂₂ClN₇O₃S (M+H)+ 512.1.

### Example 113

### 5-Chloro-N-((1-(4-(2-oxopyridin-1(2H)-yl)-2-(piperazine-1-carbonyl)phenyl)-1H-1,2,3-triazol-4-yl)methyl)thiophene-2-carboxamide (113)

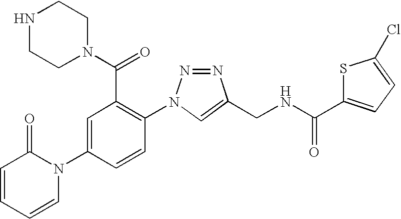

The title compound was prepared using a similar procedure as described in Example 101. MS found for C₂₄H₂₂ClN₇O₃S (M+H)+ 524.1, 526.1 (Cl pattern).

### Example 114

### 5-Chloro-N-((1-(2-(2-oxopiperazine-4-carbonyl)-4-(2-oxopyridin-1(2H)-yl)phenyl)-1H-1,2,3-triazol-4-yl)methyl)thiophene-2-carboxamide (114)

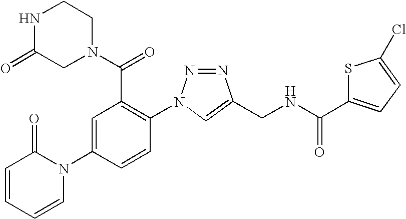

The title compound was prepared using a similar procedure as described in Example 101. MS found for C₂₄H₂₀ClN₇O₄S (M+H)+ 538.1, 540.1 (Cl pattern).

### Example 115

### 5-Chloro-N-((1-(2-(4-hydroxypiperidine-1-carbonyl)-4-(2-oxopyridin-1(2H)-yl)phenyl)-1H-1,2,3-triazol-4-yl)methyl)thiophene-2-carboxamide (115)

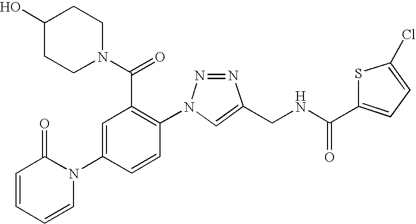

The title compound was prepared using a similar procedure as described in Example 101. MS found for C₂₅H₂₃ClN₆O₄S (M+H)+ 539.1.

### Example 116

### 1-(2-(4-((5-Chlorothiophene-2-carboxamido)methyl)-1H-1,2,3-triazol-1-yl)-5-(2-oxopyridin-1(2H)-yl)benzoyl)piperidine-4-carboxamide (116)

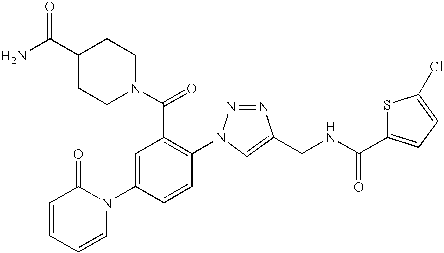

The title compound was prepared using a similar procedure as described in Example 101. MS found for C₂₆H₂₄ClN₇O₄S (M+H)+ 566.1.

### Example 117

### N-((1-(2-(((1H-Tetrazol-5-yl)methyl)carbamoyl)-4-(2-oxopyridin-1(2H)-yl)phenyl)-1H-1,2,3-triazol-4-yl)methyl)-5-chlorothiophene-2-carboxamide (117)

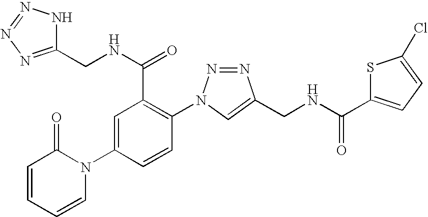

The title compound was prepared using a similar procedure as described in Example 101. MS found for C₂₂H₁₇ClN₁₀O₃S (M+H)+ 537.1, 539.1 (Cl pattern).

### Example 118

### 5-Chloro-N-((1-(4-(2-oxopyridin-1(2H)-yl)-2-(pyridin-4-ylcarbamoyl)phenyl)-1H-1,2,3-triazol-4-yl)methyl)thiophene-2-carboxamide (118)

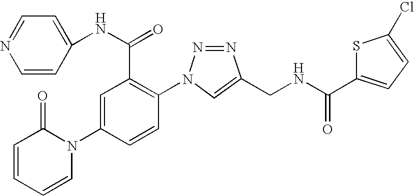

The title compound was prepared using a similar procedure as described in Example 101. MS found for C₂₅H₁₈ClN₇O₃S (M+H)+ 532.1, 534.1 (Cl pattern).

### Example 119

### 5-Chloro-N-((1-(2-(hydroxymethyl)-4-(2-oxopyridin-1(2H)-yl)phenyl)-1H-1,2,3-triazol-4-yl)methyl)thiophene-2-carboxamide (119)

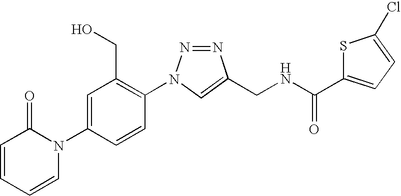

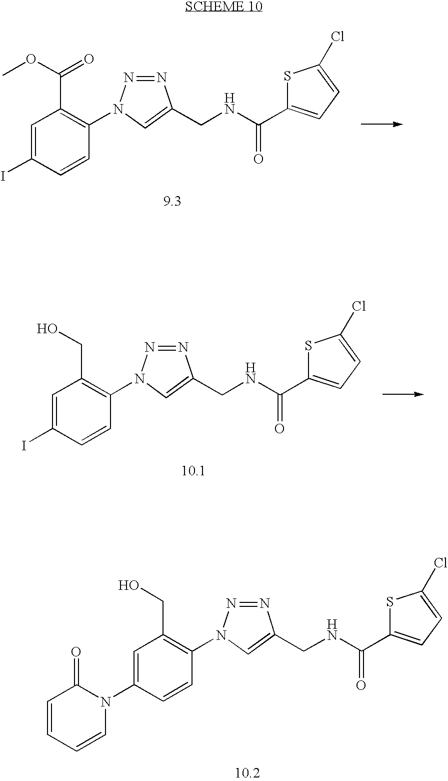

Step 1:

To a solution of compound 9.3 (1.61 g, 3.2 mmol) in 60 mL anhydrous THF was added lithium borohydride (2.0 M in THF, 4.8 mL, 9.6 mmol) dropwise. The mixture was stirred at RT overnight. To it was then added 2N HCl and 500 mL ethyl acetate. The organic phase was separated and washed with brine twice. It was dried, concentrated, and purified using flash column to afford compound 10.1 in quantitative yield. MS found for C₁₅H₁₂ClIN₄O₂S (M+H)+ 475.0, 477.0 (Cl pattern).

Step 2:

Compound 10.1 (128 mg, 0.27 mmol) was dissolved in 5 mL DMSO in a sealed tube. To it were added 2-hydroxypyridine (51 mg, 0.54 mmol), 8-hydroxypyridine (16 mg, 0.11 mmol), cesium carbonate (176 mg, 0.54 mmol) and CuI (21 mg, 0.11 mmol). The mixture was stirred at 120° C. for 17 hrs. It was then directly subjected to reverse phase preparative HPLC to isolate the title compound 10.2. MS found for C₂₀H₁₆ClN₅O₃S (M+H)+ 442.1, 444.1 (Cl pattern).

### Example 120

### N-((1-(2-(Aminomethyl)-4-(2-oxopyridin-1(2H)-yl)phenyl)-1H-1,2,3-triazol-4-yl)methyl)-5-chlorothiophene-2-carboxamide (120)

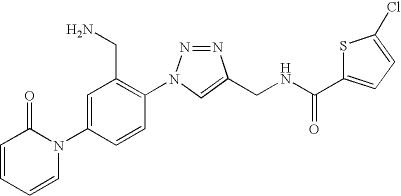

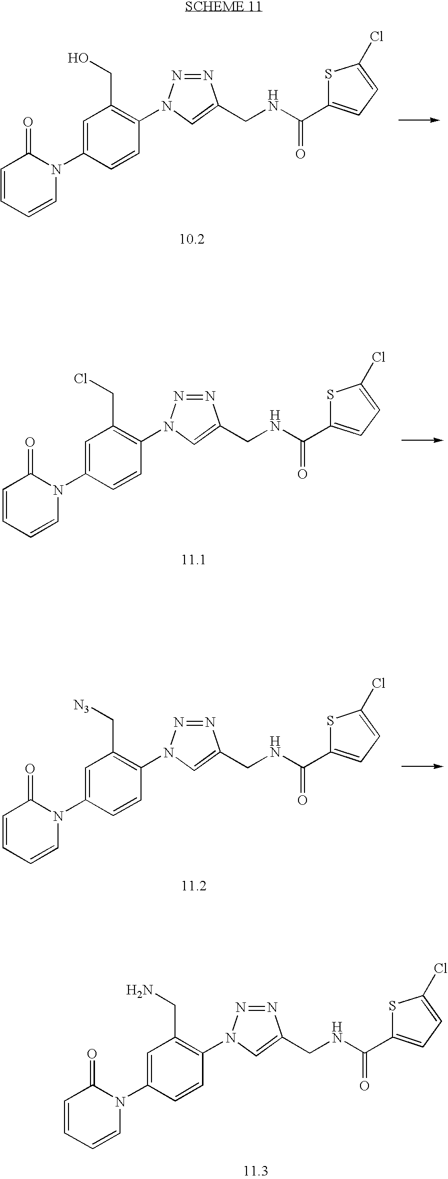

Step 1:

To a solution of compound 10.2 (10 mg, 0.022 mmol) in 2 mL anhydrous acetonitrile was added 0.5 mL thionyl chloride. The mixture was stirred at RT for 20 min. To it was added 20 mL methanol. The mixture was concentrated in vacuo to afford crude compound 11.1. MS found for C₂₀H₁₅Cl₂N₅O₂S (M+H)+ 460.0, 462.0 (Cl pattern).

Step 2:

To a solution of crude compound 11.1 in 3 mL DMSO was added sodium azide (10 mg). The mixture was stirred at RT for 2 hrs and directly subjected to reverse phase preparative HPLC to isolate compound 11.2 in quantitative yield. MS found for C₂₀H₁₅ClN₈O₂S (M+H)+ 467.1, 469.1 (Cl pattern).

Step 3:

Compound 11.2 (10 mg, 0.22 mmol) was dissolved in 4 mL ethanol and 2 mL acetic acid. It was treated with iron powder (10 eq) at 100° C. for 15 min. The mixture was dilute with acetonitrile, filtered through a celite bed, concentrated and subjected to reverse phase preparative HPLC to isolate the title compound 11.3. MS found for C₂₀H₁₇ClN₆O₂S (M+H)+ 441.1, 443.1 (Cl pattern).

### Example 121

### 5-Chloro-N-((1-(2-((dimethylamino)methyl)-4-(2-oxopyridin-1(2H)-yl)phenyl)-1H-1,2,3-triazol-4-yl)methyl)thiophene-2-carboxamide (121)

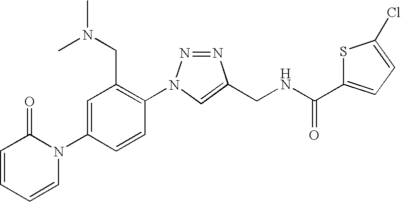

The title compound was prepared using a procedure similar to that used for preparing compound 11.2 as described in Example 120. MS found for C₂₂H₂₁ClN₆O₂S (M+H)+ 469.1, 471.1 (Cl pattern).

### Example 122

### 5-Chloro-N-((1-(4-(2-oxopyridin-1(2H)-yl)-2-(piperidin-1-ylmethyl)phenyl)-1H-1,2,3-triazol-4-yl)methyl)thiophene-2-carboxamide (122)

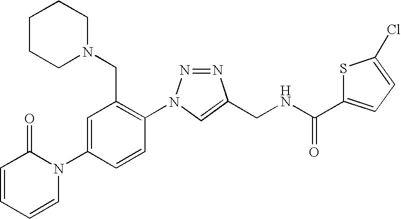

The title compound was prepared using a procedure similar to that used for preparing compound 11.2 as described in Example 120. MS found for C₂₅H₂₅ClN₆O₂S (M+H)+ 509.1, 511.1 (Cl pattern).

### Example 123

### 5-Chloro-N-((1-(2-(methylthiomethyl)-4-(2-oxopyridin-1(2H)-yl)phenyl)-1H-1,2,3-triazol-4-yl)methyl)thiophene-2-carboxamide (123)

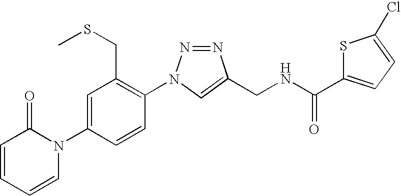

The title compound was prepared using a procedure similar to that used to prepare compound 11.2 as described in Example 120. MS found for C₂₁H₁₈ClN₅O₂S₂ (M+H)+ 472.1, 474.1 (Cl pattern).

### Example 124

### 5-Chloro-N-((1-(2-(methylsulfonylmethyl)-4-(2-oxopyridin-1(2H)-yl)phenyl)-1H-1,2,3-triazol-4-yl)methyl)thiophene-2-carboxamide (124)

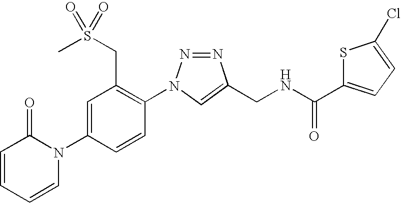

The title compound was prepared from 5-chloro-N-((1-(2-(methylthiomethyl)-4-(2-oxopyridin-1(2H)-yl)phenyl)-1H-1,2,3-triazol-4-yl)methyl)thiophene-2-carboxamide using a similar procedure as described in Example 12. MS found for C₂₁H₁₈ClN₅O₄S₂ (M+H)+ 504.0, 506.0 (Cl pattern).

### Example 125

### 5-Chloro-N-((1-(4-(2-oxopyridin-1(2H)-yl)-2-(pyridin-4-yl)phenyl)-1H-1,2,3-triazol-4-yl)methyl)thiophene-2-carboxamide (125)

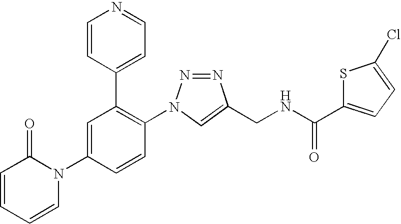

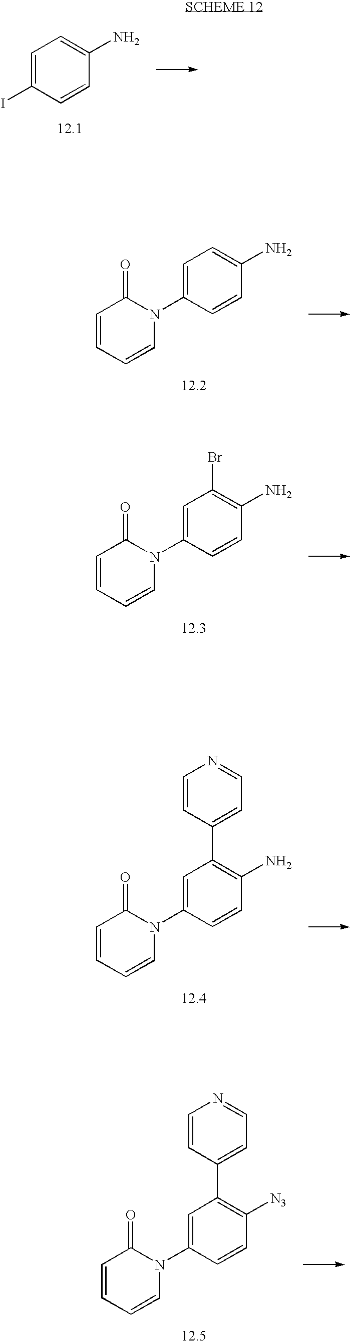

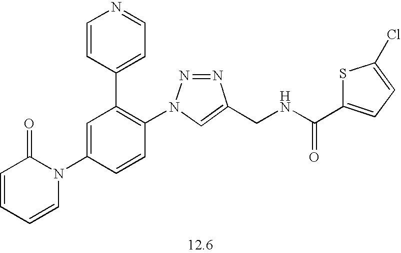

Step 1:

4-Iodoaniline (12.1, 10.0 g, 45.6 mmol) was dissolved in 80 mL dioxane and 40 mL DMSO. To it were added 2-hydroxypyridine (8.68 g, 91.2 mmol), 8-hydroxyquinoline (1.32 g, 9.12 mmol), cesium carbonate (30.0 g, 91.2 mmol) and CuI (1.73 g, 9.12 mmol). The mixture was stirred at 120° C. for 6 hrs. It was filtered, concentrated, and taken into 800 mL chloroform. The organic solution washed with brine twice and concentrated to its ¼ volume. The solid crashed out was isolated by filtration, washed with cold DCM, and dried in vacuo to give compound 12.2 (4.62 g, 54%). MS found for C₁₁H₁₀N₂O (M+H)+ 187.1.

Step 2:

To a solution of compound 12.2 (7.44 g, 40 mmol) in 100 mL DMF was added NBS (14.2 g, 80 mmol) in small portions. The mixture was stirred at RT overnight. It was diluted with 1000 mL ethyl acetate, washed with brine three times, dried, concentrated and subjected to flash column chromatography using 5% methanol in DCM to isolate compound 12.3 (7.20 g, 68%). MS found for C₁₁H₉BrN₂O (M+H)+ 265.0, 267.0.

Step 3:

Compound 12.3 (400 mg, 1.5 mmol), 4-pyridineboronic acid (185 mg, 1.5 mmol) and cesium carbonate (1.47 g, 4.5 mmol) were stirred in a mixture 2 mL n-butanol, 8 mL toluene and 4 mL water. The mixture was degassed by argon stream for 5 min. Pd(Ph₃P)₄ (350 mg, 0.3 mmol) was then added and the mixture was stirred under argon at 80° C. overnight. It was diluted with 150 mL chloroform, washed with sat sodium carbonate aq solution twice, dried, concentrated and purified using flash column with 10% methanol in DCM to give compound 12.4 (86 mg, 22%). MS found for C₁₆H₁₃N₃O (M+H)+ 264.1.

Step 4:

To a solution of compound 12.4 (86 mg, 0.33 mmol) in 5 mL TFA in ice bath was added sodium nitrite (34 mg, 0.49 mmol). The mixture was stirred in ice bath for 30 min. To it was added an ice-cold solution of sodium azide (65 mg, 1.0 mmol) in 1 mL water. The mixture was stirred for 2 hrs and directly subjected to reverse phase preparative HPLC to give compound 12.5 in a quantitative yield. MS found for C₁₆H₁₁N₅O (M+H)+ 290.1.

Step 5:

Compound 12.5 from the previous step was dissolved in 10 mL methanol. To it were added compound 1.4 (140 mg, 0.70 mmol), DBU (105 μL, 0.70 mmol) and CuI (133 mg, 0.70 mmol). The mixture was stirred overnight, diluted with acetonitrile, filtered through a celite bed, concentrated and subjected to reverse phase preparative HPLC to isolated the title compound 12.6. MS found for C₂₄H₁₇ClN₆O₂S (M+H)+ 489.1, 491.1 (Cl pattern).

### Example 126

### 5-Chloro-N-((1-(4-(2-oxopyridin-1(2H)-yl)-2-(pyridin-3-yl)phenyl)-1H-1,2,3-triazol-4-yl)methyl)thiophene-2-carboxamide (126)

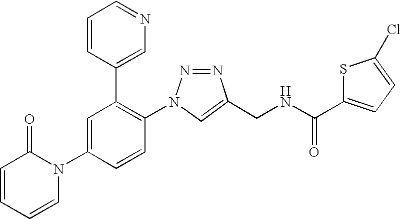

The title compound was prepared using a similar procedure as described in Example 125. MS found for C₂₄H₁₇ClN₆O₂S (M+H)+ 489.1, 491.1 (Cl pattern).

### Example 127

### 5-Chloro-N-((1-(4-(2-oxopyridin-1(2H)-yl)-2-(pyrimidin-5-yl)phenyl)-1H-1,2,3-triazol-4-yl)methyl)thiophene-2-carboxamide (127)

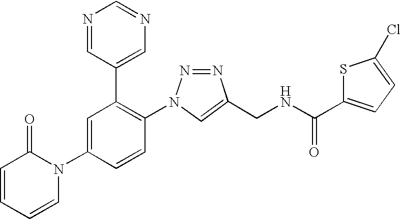

The title compound was prepared using a similar procedure as described in Example 125. MS found for C₂₃H₁₆ClN₇O₂S (M+H)+ 490.1, 492.1 (Cl pattern).

### Example 128

### 5-Chloro-N-((1-(4-(2-oxopyridin-1(2H)-yl)-2-(1H-pyrazol-3-yl)phenyl)-1H-1,2,3-triazol-4-yl)methyl)thiophene-2-carboxamide (128)

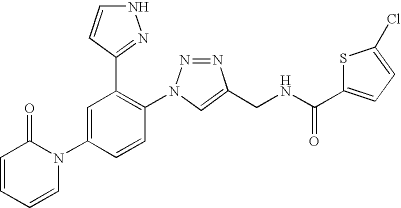

The title compound was prepared using a similar procedure as described in Example 125. MS found for C₂₂H₁₆ClN₇O₂S (M+H)+ 478.1, 480.1 (Cl pattern).

### Example 129

### 5-Chloro-N-((1-(4-(2-oxopyridin-1(2H)-yl)-2-((4-aminophenyl)phenyl)-1H-1,2,3-triazol-4-yl)methyl)thiophene-2-carboxamide (129)

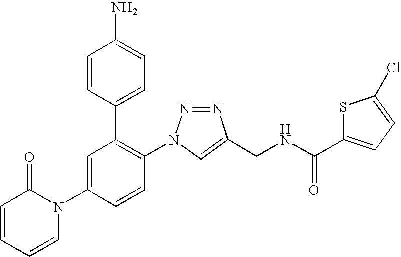

The title compound was prepared using a similar procedure as described in Example 125. MS found for C₂₅H₁₉ClN₆O₂S (M+H)+ 503.1, 505.1 (Cl pattern).

### Example 130

### 5-Chloro-N-((1-(4-(2-oxopyridin-1(2H)-yl)-2-((4-hydroxyphenyl)phenyl)-1H-1,2,3-triazol-4-yl)methyl)thiophene-2-carboxamide (130)

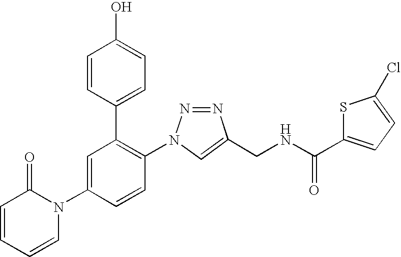

The title compound was prepared using a similar procedure as described in Example 125. MS found for C₂₅H₁₈ClN₅O₃S (M+H)+ 504.1, 506.1 (Cl pattern).

### Example 131

### 5-Chloro-N-((1-(4-(2-oxopyridin-1(2H)-yl)-2-((3-aminophenyl)phenyl)-1H-1,2,3-triazol-4-yl)methyl)thiophene-2-carboxamide (131)

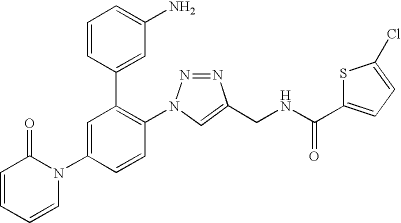

The title compound was prepared using a similar procedure as described in Example 125. MS found for C₂₅H₁₉ClN₆O₂S (M+H)+ 503.1, 505.1 (Cl pattern).

### Example 132

### 5-Chloro-N-((1-(4-(2-oxopyridin-1(2H)-yl)-2-((3-hydroxyphenyl)phenyl)-1H-1,2,3-triazol-4-yl)methyl)thiophene-2-carboxamide (132)

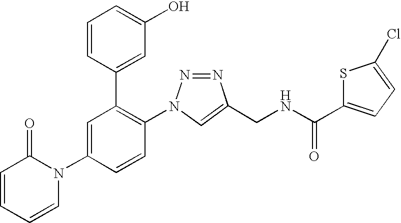

The title compound was prepared using a similar procedure as described in Example 125. MS found for C₂₅H₁₈ClN₅O₃S (M+H)+ 504.1, 506.1 (Cl pattern).

### Example 133

### 5-Chloro-N-((1-(2-(2-chloropyridin-4-yl)-4-(2-oxopyridin-1(2H)-yl)phenyl)-1H-1,2,3-triazol-4-yl)methyl)thiophene-2-carboxamide (133)

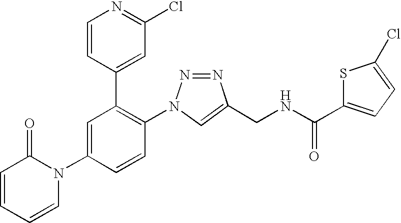

The title compound was prepared using a similar procedure as described in Example 125. MS found for C₂₄H₁₆Cl₂N₆O₂S (M+H)+ 523.0, 525.0 (Cl pattern).

### Example 134

### 5-Chloro-N-((1-(2-(2-fluoropyridin-4-yl)-4-(2-oxopyridin-1(2H)-yl)phenyl)-1H-1,2,3-triazol-4-yl)methyl)thiophene-2-carboxamide (134)

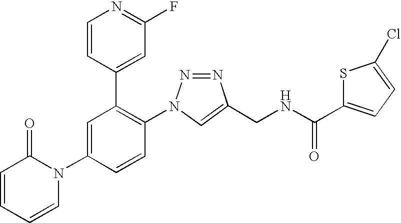

The title compound was prepared using a similar procedure as described in Example 125. MS found for C₂₄H₁₆ClFN₆O₂S (M+H)+ 507.1, 509.1 (Cl pattern).

### Example 135

### 5-Chloro-N-((1-(2-(6-chloropyridin-3-yl)-4-(2-oxopyridin-1(2H)-yl)phenyl)-1H-1,2,3-triazol-4-yl)methyl)thiophene-2-carboxamide (135)

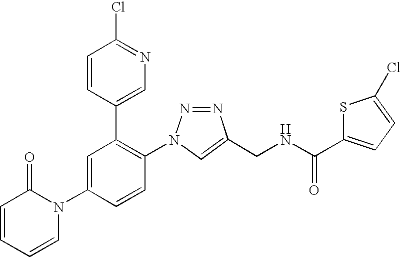

The title compound was prepared using a similar procedure as described in Example 125. MS found for C₂₄H₁₆Cl₂N₆O₂S (M+H)+ 523.0, 525.0 (Cl pattern).

### Example 136

### 5-Chloro-N-((1-(2-(6-fluoropyridin-3-yl)-4-(2-oxopyridin-1(2H)-yl)phenyl)-1H-1,2,3-triazol-4-yl)methyl)thiophene-2-carboxamide (136)

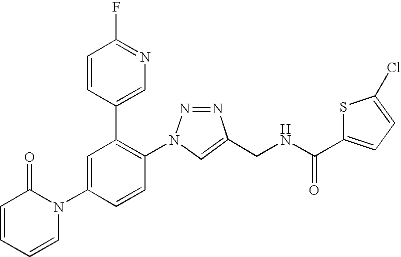

The title compound was prepared using a similar procedure as described in Example 125. MS found for C₂₄H₁₆ClFN₆O₂S (M+H)+ 507.1, 509.1 (Cl pattern).

### Example 137

### 5-Chloro-N-((1-(2H)=yl)phenyl)=1H-1,2,3-triazol-4-yl)methyl)thiophene-2-carboxamide (137)

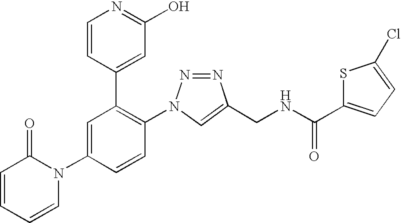

5-Chloro-N-((1-(2-(2-fluoropyridin-4-yl)-4-(2-oxopyridin-1(2H)-yl)phenyl)-1H-1,2,3-triazol-4-yl)methyl)thiophene-2-carboxamide (10 mg, 0.02 mmol) was dissolved in 2 mL DMSO and 2 mL water in a sealed tube. To it was added cesium carbonate (100 mg, 0.3 mmol) and the mixture was stirred overnight at 120° C. It was directly subjected to reverse phase preparative HPLC to isolate the title compound. MS found for C₂₄H₁₇ClN₆O₃S (M+H)+ 505.1, 507.1 (Cl pattern).

### Example 138

### 5-Chloro-N-((1-(2-(2-methoxypyridin-4-yl)-4-(2-oxopyridin-1(2H)-yl)phenyl)-1H-1,2,3-triazol-4-yl)methyl)thiophene-2-carboxamide (138)

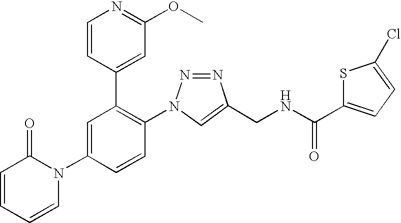

The title compound was prepared using a similar procedure as described in Example 125. MS found for C₂₅H₁₉ClN₆O₃S (M+H)+ 519.1.

### Example 139

### N-((1-(2-(2-Aminopyridin-4-yl)-4-(2-oxopyridin-1(2H)-yl)phenyl)-1H-1,2,3-triazol-4-yl)methyl)-5-chlorothiophene-2-carboxamide (139)

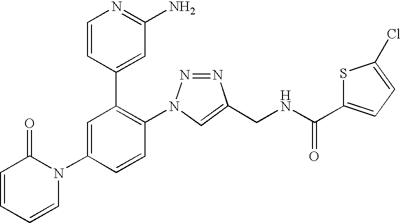

5-Chloro-N-((1-(2-(2-fluoropyridin-4-yl)-4-(2-oxopyridin-1(2H)-yl)phenyl)-1H-1,2,3-triazol-4-yl)methyl)thiophene-2-carboxamide (22 mg, 0.04 mmol) was dissolved in 2.5 mL DMSO in a sealed tube. To it was added sodium azide (26 mg, 0.4 mmol) and the mixture was stirred overnight at 120° C. It was diluted with 100 mL chloroform and washed with brine twice. The organic phase was dried, concentrated in vacuo, dissolved in 2 mL ethanol and 2 mL acetic acid. The mixture was treated with iron powder (20 mg) at 100° C. for 3 hrs, filtered and subjected to reverse phase preparative HPLC to isolate the title compound. MS found for C₂₄H₁₈ClN₇O₂S (M+H)+ 504.1, 506.1 (Cl pattern).

### Example 140

### 5-Chloro-N-((1-(2-(2-(dimethylamino)pyridin-4-yl)-4-(2-oxopyridin-1(2H)-yl)phenyl)-1H-1,2,3-triazol-4-yl)methyl)thiophene-2-carboxamide (140)

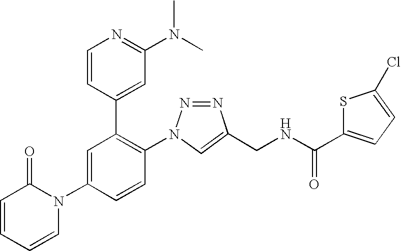

5-Chloro-N-((1-(2-(2-fluoropyridin-4-yl)-4-(2-oxopyridin-1(2H)-yl)phenyl)-1H-1,2,3-triazol-4-yl)methyl)thiophene-2-carboxamide (22 mg, 0.04 mmol) was dissolved in 3 mL DMSO in a sealed tube. To it was added dimethylamine (2 M in THF, 0.2 mL, 0.4 mmol) and the mixture was stirred overnight at 120° C. It was concentrated and directly subjected to reverse phase preparative HPLC to isolate the title compound. MS found for C₂₆H₂₂ClN₇O₂S (M+H)+ 532.1, 534.1 (Cl pattern).

### Example 141

### 5-Chloro-N-((1-(2-(2-(methylamino)pyridin-4-yl)-4-(2-oxopyridin-1(2H)-yl)phenyl)-1H-1,2,3-triazol-4-yl)methyl)thiophene-2-carboxamide (141)

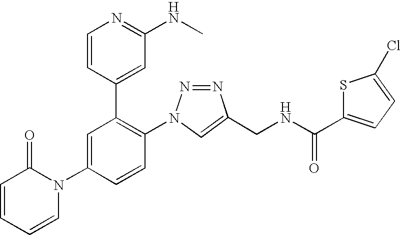

The title compound was prepared using a similar procedure as described in Example 140. MS found for C₂₅H₂₀ClN₇O₂S (M+H)+ 518.1, 520.1 (Cl pattern).

### Example 142

### 5-Chloro-N-((1-(2-(6-hydroxypyridin-3-yl)-4-(2-oxopyridin-1(2H)-yl)phenyl)-1H-1,2,3-triazol-4-yl)methyl)thiophene-2-carboxamide (142)

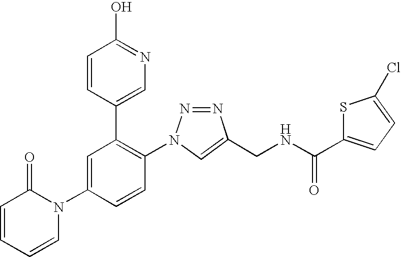

The title compound was prepared using a similar procedure as described in Example 137 from 5-chloro-N-((1-(2-(6-fluoropyridin-3-yl)-4-(2-oxopyridin-1(2H)-yl)phenyl)-1H-1,2,3-triazol-4-yl)methyl)thiophene-2-carboxamide (prepared as shown in Example 136). MS found for C₂₄H₁₇ClN₆O₃S (M+H)+ 505.1, 507.1 (Cl pattern).

### Example 143

### 5-Chloro-N-((1-(2-(6-methoxypyridin-3-yl)-4-(2-oxopyridin-1(2H)-yl)phenyl)-1H-1,2,3-triazol-4-yl)methyl)thiophene-2-carboxamide (143)

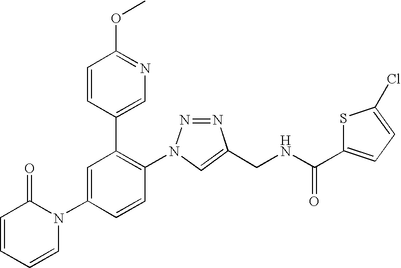

The title compound was prepared using a similar procedure as described in Example 125. MS found for C₂₅H₁₉ClN₆O₃S (M+H)+ 519.1.

### Example 144

### N-((1-(2-(6-Aminopyridin-3-yl)-4-(2-oxopyridin-1(2H)-yl)phenyl)-1H-1,2,3-triazol-4-yl)methyl)-5-chlorothiophene-2-carboxamide (144)

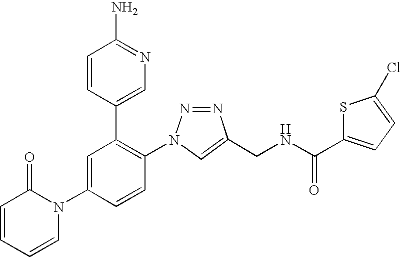

The title compound was prepared using a similar procedure as described in Example 139. MS found for C₂₄H₁₈ClN₇O₂S (M+H)+ 504.1, 506.1 (Cl pattern).

### Example 145

### 5-Chloro-N-((1-(2-(6-(dimethylamino)pyridin-3-yl)-4-(2-oxopyridin-1(2H)-yl)phenyl)-1H-1,2,3-triazol-4-yl)methyl)thiophene-2-carboxamide (145)

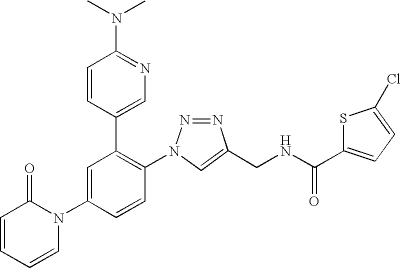

The title compound was prepared using a similar procedure as described in Example 140. MS found for C₂₆H₂₂ClN₇O₂S (M+H)+ 532.1, 534.1 (Cl pattern).

### Example 146

### 5-Chloro-N-((1-(2-(6-(methylamino)pyridin-3-yl)-4-(2-oxopyridin-1(2H)-yl)phenyl)-1H-1,2,3-triazol-4-yl)methyl)thiophene-2-carboxamide (146)

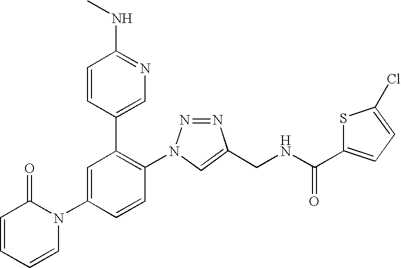

The title compound was prepared using a similar procedure as described in Example 141. MS found for C₂₅H₂₀ClN₇O₂S (M+H)+ 518.1, 520.1 (Cl pattern).

### Example 147

### N-((1-(2-(3-Amino-3-oxopropylcarbamoyl)-4-(2-oxopyridin-1(2H)-yl)phenyl)-1H-1,2,3-triazol-4-yl)methyl)-5-chlorothiophene-2-carboxamide (147)

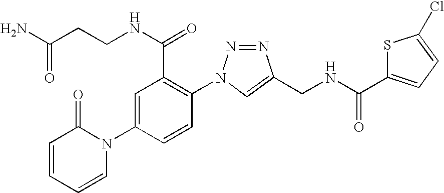

The title compound was prepared using a similar procedure as described in Example 101. MS found for C₂₃H₂₀ClN₇O₄S (M+H)+ 526.1, 528.1 (Cl pattern).

### Example 148

### N-((1-(2-(2-(1H-Imidazol-1-yl)ethoxy)-4-(2-oxopyridin-1(2H)-yl)phenyl)-1H-1,2,3-triazol-4-yl)methyl)-5-chlorothiophene-2-carboxamide (148)

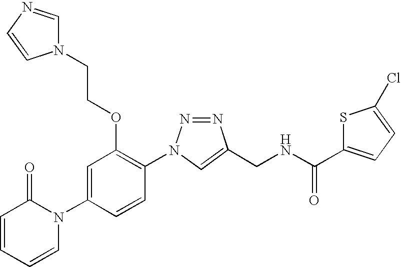

The title compound was prepared using a similar procedure as described in Example 165 below. MS found for C₂₄H₂₀ClN₇O₃S (M+H)+ 522.1, 524.1 (Cl pattern).

### Example 149

### 5-Chloro-N-((1-(4-(2-oxopyridin-1(2H)-yl)-2-(2-(pyridin-4-yl)ethoxy)phenyl)-1H-1,2,3-triazol-4-yl)methyl)thiophene-2-carboxamide (149)

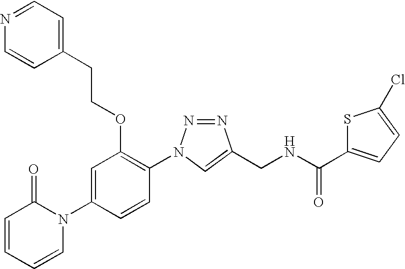

The title compound was prepared using a similar procedure as described in Example 165 below. MS found for C₂₆H₂₁ClN₆O₃S (M+H)+ 533.1, 535.1 (Cl pattern).

### Example 150

### 3-(2-(4-((5-Chlorothiophene-2-carboxamido)methyl)-1H-1,2,3-triazol-1-yl)-5-(2-oxopyridin-1(2H)-yl)phenoxy)propyl morpholine-4-carboxylate (150)

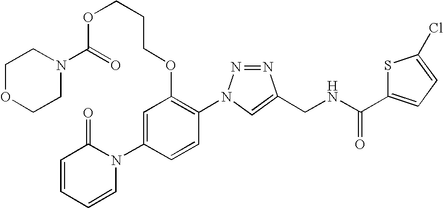

The title compound was prepared using a similar procedure as described in Example 165 below. MS found for C₂₇H₂₇ClN₆O₆S (M+H)+ 599.1, 601.1 (Cl pattern).

### Example 151

### 5-Chloro-N-((1-(4-(2-oxopyridin-1(2H)-yl)-2-(pyridin-2-yloxy)phenyl)-1H-1,2,3-triazol-4-yl)methyl)thiophene-2-carboxamide (151)

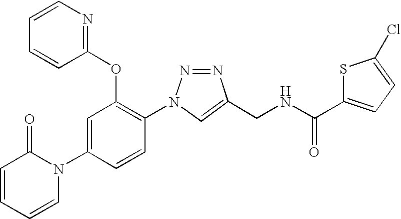

The title compound was prepared using a similar procedure as described in Example 57. MS found for C₂₄H₁₇ClN₆O₃S (M+H)+ 505.1, 507.1 (Cl pattern).

### Example 152

### 2-(2-(4-((5-Chlorothiophene-2-carboxamido)methyl)-5-iodo-1H-1,2,3-triazol-1-yl)-5-(2-oxopyridin-1(2H)-yl)phenoxy)acetic acid (152)

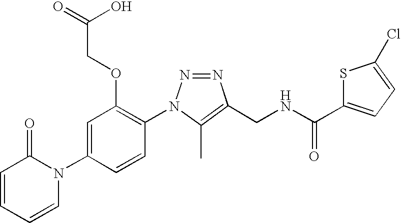

The title compound was prepared by a similar procedure as described in the above Examples. MS found for C₂₁H₁₅ClIN₅O₅S (M+H)+ 612.0.

### Example 153

### 5-Chloro-N-((1-(2-(3-hydroxypropoxy)-4-(2-oxopyridin-1(2H)-yl)phenyl)-5-iodo-1H-1,2,3-triazol-4-yl)methyl)thiophene-2-carboxamide (153)

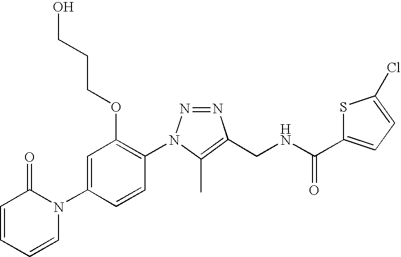

The title compound was prepared by a procedure similar to that described in the above Examples. MS found for C₂₃H₂₁ClIN₅O₄S (M+H)+ 612.0.

### Example 154

### 5-Chloro-N-((1-(2-((R)-2,3-dihydroxypropoxy)-4-(2-oxopyridin-1(2H)-yl)phenyl)-5-iodo-1H-1,2,3-triazol-4-yl)methyl)thiophene-2-carboxamide (154)

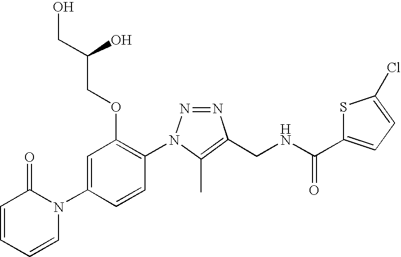

The title compound was prepared by a procedure similar to that described in the above Examples. MS found for C₂₂H₁₉ClIN₅O₅S (M+H)+ 628.1.

### Example 155

### 5-Chloro-N-((1-(2-((S)-2,3-dihydroxypropoxy)-4-(2-oxopyridin-1(2H)-yl)phenyl)-5-iodo-1H-1,2,3-triazol-4-yl)methyl)thiophene-2-carboxamide (155)

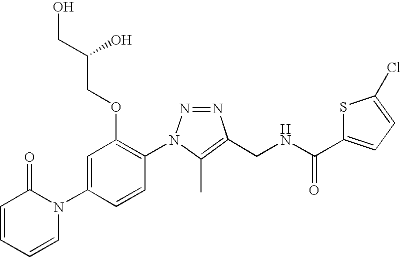

The title compound was prepared by a procedure similar to that described in the above Examples. MS found for C₂₂H₁₉ClIN₅O₅S (M+H)+ 628.1.

### Example 156

### N-((1-(2-(2-(1H-Pyrazol-1-yl)ethoxy)-4-(2-oxopyridin-1(2H)-yl)phenyl)-1H-1,2,3-triazol-4-yl)methyl)-5-chlorothiophene-2-carboxamide (156)

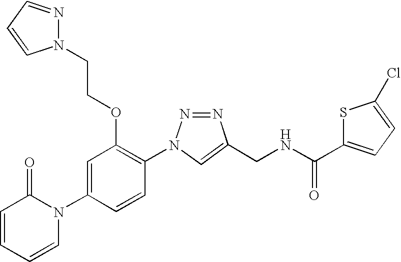

The title compound was prepared using a similar procedure as described in Example 165 below. MS found for C₂₄H₂₀ClN₇O₃S (M+H)+ 522.1, 524.1 (Cl pattern).

### Example 157

### 5-Chloro-N-((1-(4-(2-oxopiperidin-1-yl)-2-thiomorpholinophenyl)-1H-1,2,3-triazol-4-yl)methyl)thiophene-2-carboxamide (157)

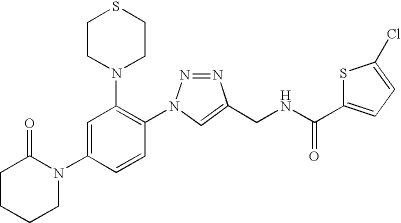

The title compound was prepared using a similar procedure as described in Example 1. MS found for C₂₃H₂₅ClN₆O₂S₂ (M+H)+ 517.1, 519.1 (Cl pattern).

### Example 158

### 5-Chloro-N-((1-(4-(2-oxopiperidin-1-yl)-2-((1,1-dioxo)thiomorpholino)phenyl)-1H-1,2,3-triazol-4-yl)methyl)thiophene-2-carboxamide (158)

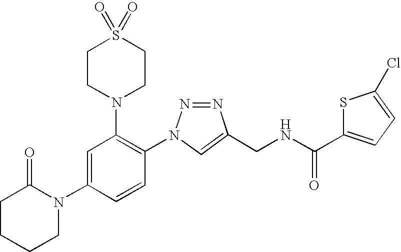

The title compound was prepared from 5-chloro-N-((1-(4-(2-oxopiperidin-1-yl)-2-thiomorpholinophenyl)-1H-1,2,3-triazol-4-yl)methyl)thiophene-2-carboxamide using a similar procedure as described in Example 12. MS found for C₂₃H₂₅ClN₆O₄S₂ (M+H)+ 549.1, 551.1 (Cl pattern).

### Example 159

### 5-Chloro-N-((1-(2-(3-oxopiperazin-1-yl)-4-(2-oxopiperidin-1-yl)phenyl)-1H-1,2,3-triazol-4-yl)methyl)thiophene-2-carboxamide (159)

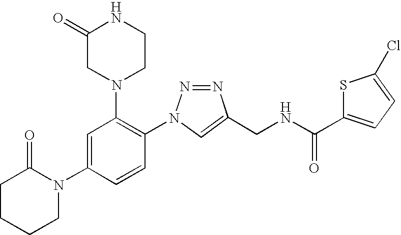

The title compound was prepared using a similar procedure as described in Example 1. MS found for C₂₃H₂₄ClN₇O₃S (M+H)+ 514.1, 516.1 (Cl pattern).

### Example 160

### 5-Chloro-N-((1-(2-(morpholinomethyl)-4-(2-oxopyridin-1(2H)-yl)phenyl)-1H-1,2,3-triazol-4-yl)methyl)thiophene-2-carboxamide (160)

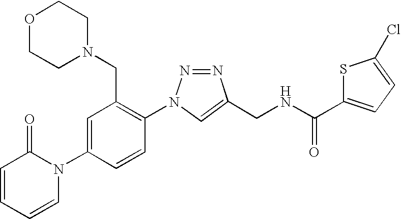

The title compound was prepared using a similar procedure as described in Example 121. MS found for C₂₄H₂₃ClN₆O₃S (M+H)+ 511.1, 513.1 (Cl pattern).

### Example 161

### 5-Chloro-N-((1-(2-((3-oxopiperazin-1-yl)methyl)-4-(2-oxopyridin-1(2H)-yl)phenyl)-1H-1,2,3-triazol-4-yl)methyl)thiophene-2-carboxamide (161)

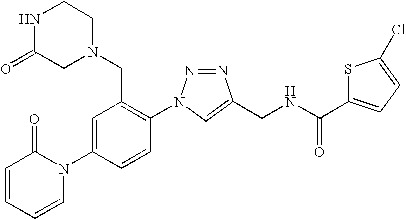

The title compound was prepared using a similar procedure as described in Example 121. MS found for C₂₄H₂₂ClN₇O₃S (M+H)+ 524.1, 526.1 (Cl pattern).

### Example 162

### 5-Chloro-N-((1-(4-(2-oxopyridin-1(2H)-yl)-2-(thiomorpholinomethyl)phenyl)-1H-1,2,3-triazol-4-yl)methyl)thiophene-2-carboxamide (162)

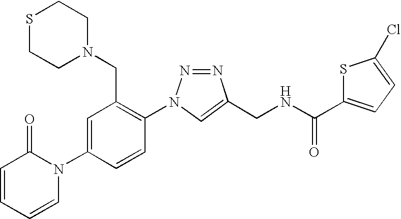

The title compound was prepared using a similar procedure as described in Example 121. MS found for C₂₄H₂₃ClN₆O₂S₂ (M+H)+ 527.1, 529.1 (Cl pattern).

### Example 163

### 5-Chloro-N-((1-(4-(2-oxopyridin-1(2H)-yl)-2-(1,1,-dioxothiomorpholinomethyl)phenyl)-1H-1,2,3-triazol-4-yl)methyl)thiophene-2-carboxamide (163)

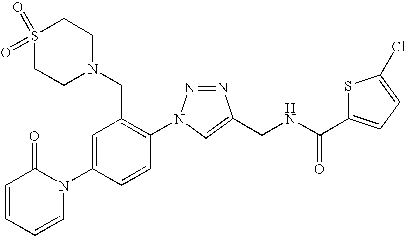

The title compound was prepared from Example 162 using a similar procedure as described in Example 12. MS found for C₂₄H₂₃ClN₆O₄S₂ (M+H)+ 559.1, 561.1 (Cl pattern).

### Example 164

### 5-Chloro-N-((1-(2-ethoxy-4-(2-oxopyridin-1(2H)-yl)phenyl)-1H-1,2,3-triazol-4-yl)methyl)thiophene-2-carboxamide (164)

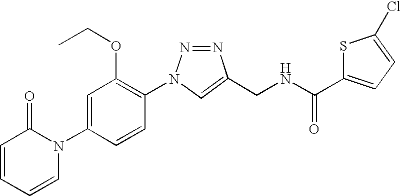

The title compound was prepared using a similar procedure as described in Example 74. MS found for C₂₁H₁₈ClN₅O₃S (M+H)+ 456.1, 458.1 (Cl pattern).

### Example 165

### 5-Chloro-N-((1-(4-(2-oxopyridin-1(2H)-yl)-2-(pyridin-3-ylmethoxy)phenyl)-1H-1,2,3-triazol-4-yl)methyl)thiophene-2-carboxamide (165)

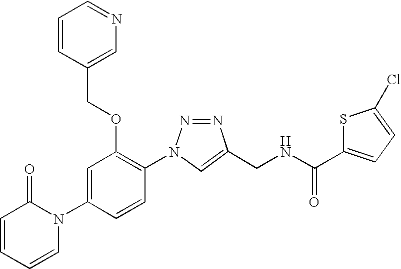

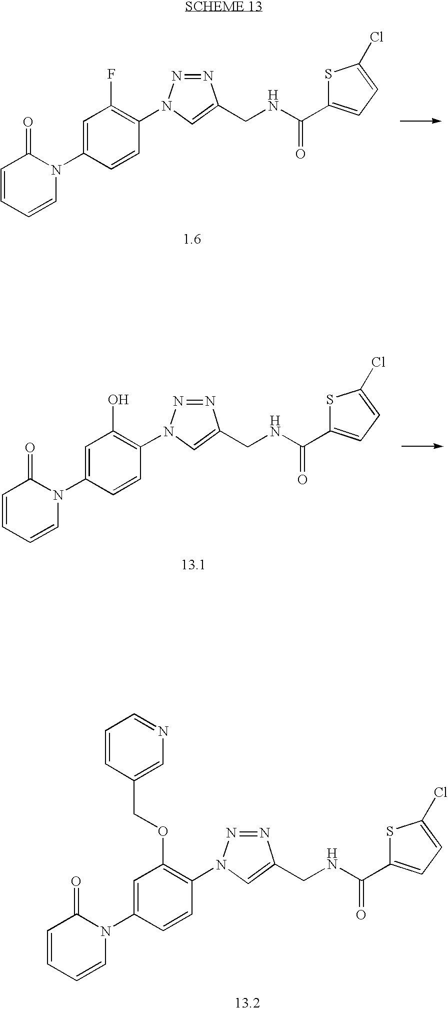

Step 1:

To a solution of compound 1.6 (6.43 g, 15 mmol) in 45 mL DMSO and 15 mL water was added sodium hydroxide (1.80 g, 45 mmol). The mixture was stirred in a sealed flask at 140° C. for 16 hrs. After it was cooled to RT, 100 mL IN sulfuric acid and 200 mL water were added carefully. The mixture was vigorously stirred and chilled in ice bath. The solid precipitate was collected using a Buchner funnel and rinsed with cold DI water, and dried in vacuo to give compound 13.1 (4.10 g, 64%). MS found for C₁₉H₁₄ClN₅O₃S (M+H)+ 428.1, 430.1 (Cl pattern).

Step 2:

Compound 13.1 (43 mg, 0.1 mmol) was dissolved in 2 mL DMSO. To it were added sodium carbonate (64 mg, 0.6 mmol) and 2-bromomethylpyridine.hydrogenbromide (51 mg, 0.2 mmol). The mixture was stirred in 60° C. bath for 30 min to afford title compound 13.2, which was isolated directly using prep HPLC as a white powder. MS found for C₂₅H₁₉ClN₆O₃S (M+H)+ 519.1, 521.1 (Cl pattern).

### Example 166

### 5-Chloro-N-((1-(4-(2-oxopyridin-1(2H)-yl)-2-(N-(pyridine-3-yl)pyridin-3-ylmethoxy)phenyl)-1H-1,2,3-triazol-4-yl)methyl)thiophene-2-carboxamide (166)

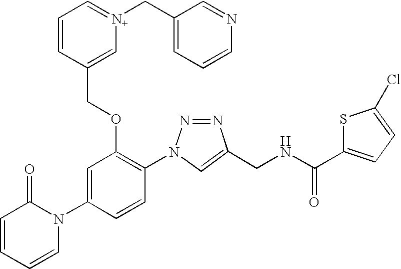

The title compound was prepared using a similar procedure as described in Example 165. MS found for C₂₅H₁₉ClN₆O₃S (M)+ 610.1.

### Example 167

### 5-Chloro-N-((1-(4-(2-oxopyridin-1(2H)-yl)-2-(pyridin-4-ylmethoxy)phenyl)-1H-1,2,3-triazol-4-yl)methyl)thiophene-2-carboxamide (167)

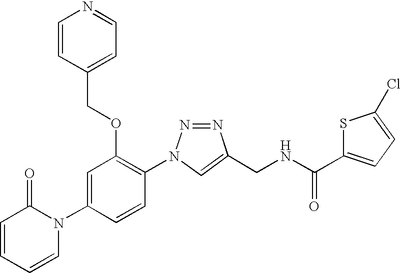

The title compound was prepared using a similar procedure as described in Example 165. MS found for C₂₅H₁₉ClN₆O₃S (M+H)+ 519.1, 521.1 (Cl pattern).

### Example 168

### 5-Chloro-N-((1-(4-(2-oxopyridin-1(2H)-yl)-2-(2-(pyridin-2-yl)ethoxy)phenyl)-1H-1,2,3-triazol-4-yl)methyl)thiophene-2-carboxamide (168)

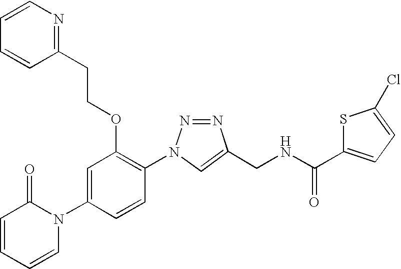

The title compound was prepared using a similar procedure as described in Example 165. MS found for C₂₆H₂₁ClN₆O₃S (M+H)+ 533.1, 535.1 (Cl pattern).

### Example 169

### 5-Chloro-N-((1-(4-(2-oxopyridin-1(2H)-yl)-2-pyridin-2-ylmethoxy)phenyl)-1H-1,2,3-triazol-4-yl)methyl)thiophene-2-carboxamide (169)

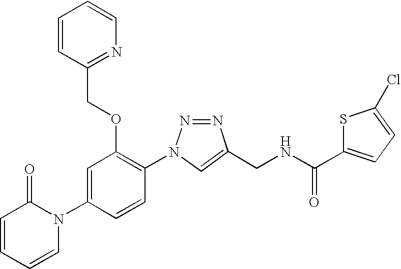

The title compound was prepared using a similar procedure as described in Example 165. MS found for C₂₅H₁₉ClN₆O₃S (M+H)+ 519.1, 521.1 (Cl pattern).

### Example 170

### 5-Chloro-N-((1-(4-(2-oxopyridin-1(2H)-yl)-2-(quinolin-2-ylmethoxy)phenyl)-1H-1,2,3-triazol-4-yl)methyl)thiophene-2-carboxamide (170)

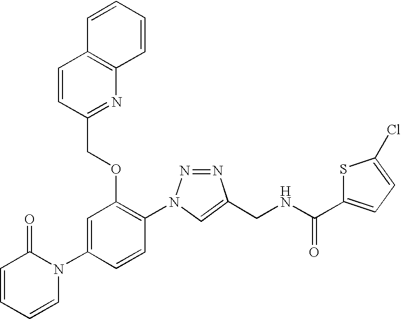

The title compound was prepared using a similar procedure as described in Example 165. MS found for C₂₉H₂₁ClN₆O₃S (M+H)+ 569.1, 571.1 (Cl pattern).

### Example 171

### 5-Chloro-N-((1-(4-(2-oxopyridin-1(2H)-yl)-2-(thiazol-4-ylmethoxy)phenyl)-1H-1,2,3-triazol-4-yl)methyl)thiophene-2-carboxamide (171)

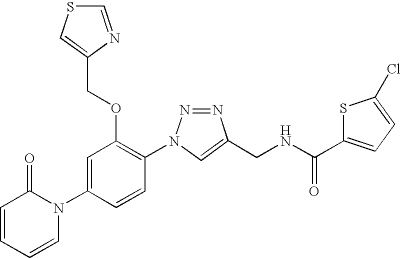

The title compound was prepared using a similar procedure as described in Example 165. MS found for C₂₃H₁₇ClN₆O₃S₂ (M+H)+ 525.1, 527.1 (Cl pattern).

### Example 172

### 5-Chloro-N-((1-(2-((2-methylthiazol-4-yl)methoxy)-4-(2-oxopyridin-1(2H)-yl)phenyl)-1H-1,2,3-triazol-4-yl)methyl)thiophene-2-carboxamide (172)

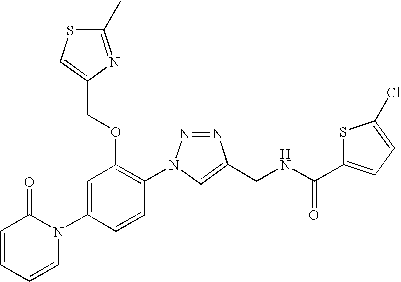

The title compound was prepared using a similar procedure as described in Example 165. MS found for C₂₄H₁₉ClN₆O₃S₂ (M+H)+ 539.1, 541.1 (Cl pattern).

### Example 173

### N-((1-(2-((1H-Benzo[d]imidazol-2-yl)methoxy)-4-(2-oxopyridin-1(2H)-yl)phenyl)-1H-1,2,3-triazol-4-yl)methyl)-5-chlorothiophene-2-carboxamide (173)

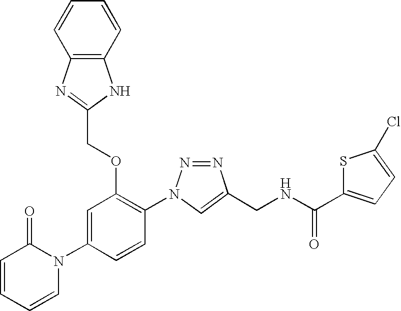

The title compound was prepared using a similar procedure as described in Example 165. MS found for C₂₇H₂₀ClN₇O₃S (M+H)+ 558.1, 560.1 (Cl pattern).

### Example 174

### 5-Chloro-N-((1-(4-(2-oxopyridin-1(2H)-yl)-2-(2-(pyridin-3-yl)ethoxy)phenyl)-1H-1,2,3-triazol-4-yl)methyl)thiophene-2-carboxamide (174)

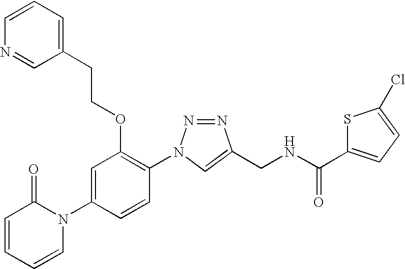

The title compound was prepared using a similar procedure as described in Example 165. MS found for C₂₆H₂₁ClN₆O₃S (M+H)+ 533.1, 535.1 (Cl pattern).

### Example 175

### N-((1-(2-((1,2,4-Oxadiazol-3-yl)methoxy)-4-(2-oxopyridin-1(2H)-yl)phenyl)-1H-1,2,3-triazol-4-yl)methyl)-5-chlorothiophene-2-carboxamide (175)

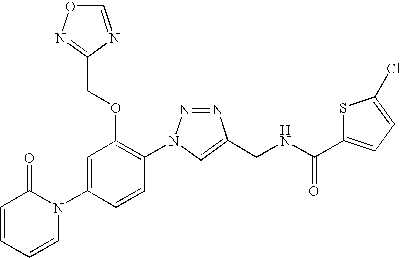

The title compound was prepared using a similar procedure as described in Example 165. MS found for C₂₂H₁₆ClN₇O₄S (M+H)+ 510.1, 512.1 (Cl pattern).

### Example 176

### 5-Chloro-N-((1-(2-((1-methyl-1H-imidazol-2-yl)methoxy)-4-(2-oxopyridin-1(2H)-yl)phenyl)-1H-1,2,3-triazol-4-yl)methyl)thiophene-2-carboxamide (176)

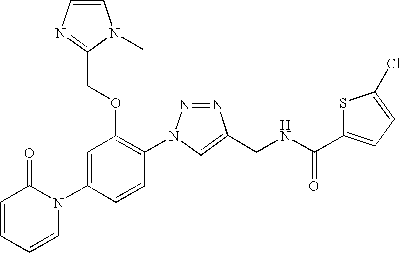

The title compound was prepared using a similar procedure as described in Example 165. MS found for C₂₄H₂₀ClN₇O₃S (M+H)+ 522.1, 524.1 (Cl pattern).

### Example 177

### N-((1-(2-((1H-Imidazol-2-yl)methoxy)-4-(2-oxopyridin-1(2H)-yl)phenyl)-1H-1,2,3-triazol-4-yl)methyl)-5-chlorothiophene-2-carboxamide (177)

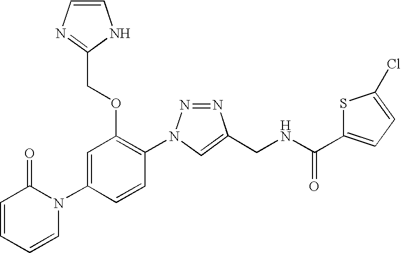

The title compound was prepared using a similar procedure as described in Example 165. MS found for C₂₃H₁₈ClN₇O₃S (M+H)+ 508.1, 510.1 (Cl pattern).

### Example 178

### N-((1-(2-((1-((1H-Imidazol-2-yl)methyl)-1H-imidazol-2-yl)methoxy)-4-(2-oxopyridin-1(2H)-yl)phenyl)-1H-1,2,3-triazol-4-yl)methyl)-5-chlorothiophene-2-carboxamide (178)

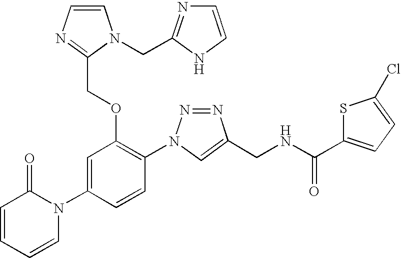

The title compound was prepared using a similar procedure as described in Example 165. MS found for C₂₇H₂₂ClN₉O₃S (M+H)+ 588.1.

### Example 179

### 5-Chloro-N-((1-(2-((5-methylisoxazol-3-yl)methoxy)-4-(2-oxopyridin-1(2H)-yl)phenyl)-1H-1,2,3-triazol-4-yl)methyl)thiophene-2-carboxamide (179)

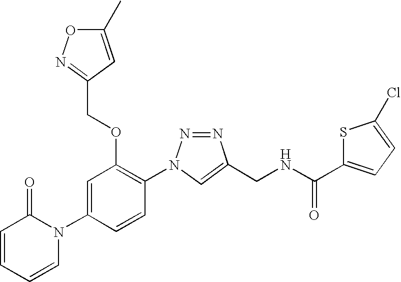

The title compound was prepared using a similar procedure as described in Example 165. MS found for C₂₄H₁₉ClN₆O₄S (M+H)+ 523.1, 525.1 (Cl pattern).

### Example 180

### N-((1-(2-(2-(1H-Pyrrol-1-yl)ethoxy)-4-(2-oxopyridin-1(2H)-yl)phenyl)-1H-1,2,3-triazol-4-yl)methyl)-5-chlorothiophene-2-carboxamide (180)

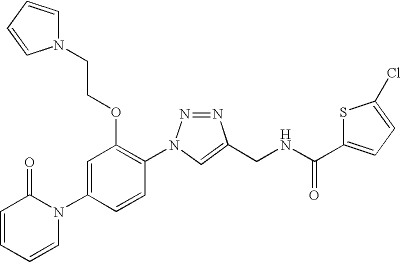

The title compound was prepared using a similar procedure as described in Example 165. MS found for C₂₅H₂₁ClN₆O₃S (M+H)+ 521.1, 523.1 (Cl pattern).

### Example 181

This example illustrates methods for evaluating the compounds of the invention, along with results obtained for such assays. The in vitro and in vivo human Factor Xa activities of the inventive compounds can be determined by various procedures known in the art, such as a test for their ability to inhibit the activity of human plasma Factor Xa. The potent affinities for human Factor Xa inhibition exhibited by the inventive compounds can be measured by an IC₅₀ value (in nM). The IC₅₀ value is the concentration (in nM) of the compound required to provide 50% inhibition of human Factor Xa proteolytic activity. The smaller the IC₅₀ value, the more active (potent) is a compound for inhibiting Factor Xa activity.

An in vitro assay for detecting and measuring inhibition activity against Factor Xa is as follows:

### IC₅₀ and Ki Determinations:

### Substrate:

The substrate S-2765 (Z-D-Arg-Gly-Arg-pNA.HCl) was obtained from Diapharma (West Chester, Ohio).

### Enzyme:

The human plasma protein factor Xa was purchased from Haematologic Technologies (Essex Junction, Vt.).

### Methods:

IC₅₀ Determinations

All assays, which are performed in 96-well microtiter plates, measure proteolytic activity of the enzyme (factor Xa) by following cleavage of a paranitroanilide peptide substrate. The assay buffer used for proteolytic assays was Tris buffered saline (20 mM Tris, 150 mM NaCl, 5 mM CaCl₂, 0.1% Bovine serum albumin (BSA), 5% Dimethly Sulfoxide (DMSO) pH 7.4). In a 96-well microtiter plate, inhibitor was serially diluted to give a range of final concentrations from 0.01 nM to 10 μM. Duplicate sets of wells were assayed and control wells without inhibitor were included. Enzyme was added to each well, (factor Xa concentration=1 nM), the plate was shaken for 5 seconds and then incubated for 5 minutes at room temperature. S-2765 was added (100 μM final) and the plate was shaken for 5 seconds (final volume in each well was 200 μl). The degree of substrate hydrolysis was measured at 405 nm on a Thermomax plate reader (Molecular Devices, Sunnyvale, Calif.) for 2 minutes. The initial velocities of substrate cleavage (mOD/min), for each range of inhibitor concentrations, were fitted to a four parameter equation using Softmax data analysis software. The parameter C, derived from the resulting curve-fit, corresponded to the concentration for half maximal inhibition (IC₅₀).

Kᵢ Determination

The assay buffer for this series of assays was Hepes buffered saline (20 mM Hepes, 150 mM NaCl, 5 mM CaCl₂, 0.1% PEG-8000, pH 7.4). In a 96-well microtiter plate, inhibitor was serially diluted in a duplicate set of wells to give a range of final concentrations from 5 μM to 3 μM. Controls without inhibitor (8 wells) were included. The enzyme, factor Xa (final concentration=1 nM) was added to the wells. The substrate S-2765 (final concentration=200 μM) was added and the degree of substrate hydrolysis was measured at 405 nm on a Thermomax plate reader for 5 minutes, using Softmax software. Initial velocities (mOD/min) were analyzed by non-linear least squares regression in the Plate Kᵢ software (BioKin Ltd, Pullman, Wash.) [Kusmic, et al., Analytical Biochemistry 281: 62-67, 2000]. The model used for fitting the inhibitor dose-response curves was the Morrison equation. An apparent Kᵢ (Ki*) was determined. The overall Kᵢ was calculated using the following equation:

$$Ki = \frac{{Ki}^{*}}{1 + \frac{[ S ]}{Km}}$$

where [S] is substrate concentration (200 μM) and Kₘ is the Michaelis constant for S-2765.

The following compounds exhibited Factor Xa IC₅₀ values of less than or equal to 100 nM: 1-6, 10-18, 20-26, 28-30, 42-45, 50-54, 56, 57, 63, 64, 67, 68, 74-76, 78-80, 82, 83, 85, 86, 88-95, 98, 99, 101-103, 109, 114, 117, 119-121, 125, 126, 133-135, 137-146, 148, 150-152, and 155-180.

The following compounds exhibited Factor Xa IC₅₀ values of greater than 100 nM and less than 500 nM: 96, 100, 104-108, 110-113, 115, 116, 118, 147, 153, and 154.

Thus, as examples, the data show that a variety of compounds of Formulas (I), (II), (Ia) and (Ib) are highly active Factor Xa inhibitors.

The present invention provides a number of embodiments. It is apparent that the examples may be altered to provide other embodiments of this invention. Therefore, it will be appreciated that the scope of this invention is to be defined by the appended claims rather than by the specific embodiments, which have been represented by way of example.

What is claimed is:

1. A compound of Formula (I) or a pharmaceutically acceptable salt, ester, or prodrug thereof: 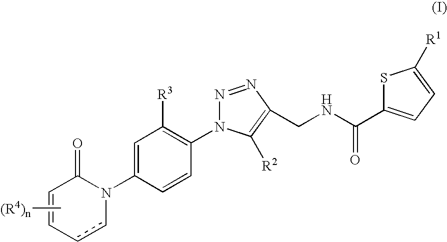
wherein
R¹ is halogen;
R² is hydrogen or halogen;
R³ is selected from the group consisting of —NO₂, —NR^{5a}R^{5b}, -L-NR^{5a}R^{5b}, —NHC(O)NR^{5a}R^{5b}, —NHC(O)R^{5c}, —NHC(O)Y, C₁₋₆ alkyl, —CO₂H, —C(O)NR^{5a}R^{5b}, —C(O)NR^{5a}Y, —C(O)NH-L-Y, OH, C₁₋₆ alkoxy, —O-L-NR^{5a}R^{b}, —O-L-O—C(O)NR^{5a}R^{5b}, —Y, —O—Y, —O-L-Y, —O-L-Y-L-Y, and —S(O)ₚR^{5c}, wherein said C₁₋₆ alkyl and C₁₋₆ alkoxy are optionally substituted with one to three substituents selected from R⁶;
R⁴ is independently selected from the group consisting of halogen, OH, —O-L-Y, —O-L-NR^{5a}R^{b}, and C₁₋₆ alkoxy optionally substituted with one to three substituents selected from R⁶;
L is C₁-C₄ alkylene;
Y is aryl, heteroaryl, or heterocyclic ring, wherein said aryl and heteroaryl are optionally substituted with one to three R⁶ and said heterocyclic ring is optionally substituted with oxo and optionally with one to three R⁶ or R⁸;
R^{5a} and R^{5b} are independently hydrogen or C₁₋₈ alkyl optionally substituted with one to three R⁶, or R^{5a} and R^{5b} together with the nitrogen atom to which they are both attached to form a 5 to 7 membered heterocyclic ring optionally having one additional ring heteroatom selected from N, NR⁶, O, or S(O)ₚ and where said ring is optionally substituted with one to three substituents selected from R⁶;
R^{5c} is C₁₋₈ alkyl optionally substituted with one to three R⁶;
R⁶ is independently selected from the group consisting of halogen, —OH, —R⁷, —OR⁷, oxo, —SR⁷, —S(O)R⁷, —S(O)₂R⁷, —SO₂NH₂, —C(O)NH₂, —C(O)R⁷, —C(NH)R⁷, —NHC(O)R⁷, —NHC(NH)R⁷, —NHC(O)NH₂, —CO₂H, —NH₂, —NHR⁷, —N(R⁷)₂;
R⁷ is independently C₁₋₆ alkyl;
R⁸ is -L-heteroaryl optionally substituted with one to three substituents selected from R⁶;
n is 0, 1, or 2;
p is 0, 1, or 2; and
the dashed lines [symbol] are independently single or double bonds;
provided that R³ is not 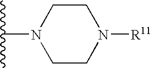
where R¹¹ is hydrogen or alkyl.

2. A compound of claim 1 having the Formula (Ia) or a pharmaceutically acceptable salt, ester, or prodrug thereof: 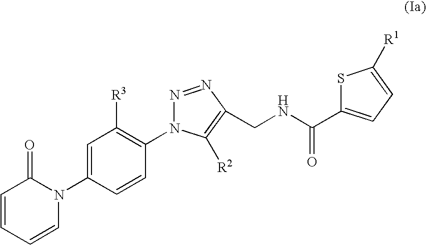
wherein R¹, R², and R³ are as previously defined for Formula (I).

3. A compound of claim 1 having the Formula (Ib) or a pharmaceutically acceptable salt, ester, or prodrug thereof: 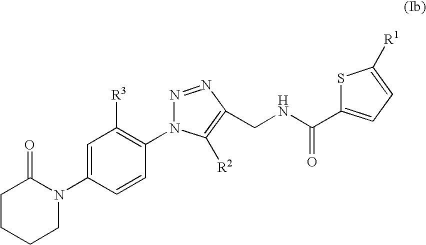
wherein R¹, R², and R³ are as previously defined for Formula (I).

4. A compound of any one of claims 1-3 wherein R¹ is chlorine.

5. A compound of any one of claims 1-3 wherein R² is hydrogen.

6. A compound of any one of claims 1-3 wherein R³ is selected from the group consisting of —NO₂, —NR^{5a}R^{5b}, -L-NR^{5a}R^{5b}, —NHC(O)NR^{5a}R^{5b}, —NHC(O)R^{5c}, —NHC(O)Y, C₁₋₆ alkyl, —CO₂H, —C(O)NR^{5a}R^{5b}, —C(O)NH-L-Y, OH, C₁₋₆ alkoxy, —O-L-NR^{5a}R^{5b}, —O-L-O—C(O)NR^{5a}R^{5b}, —Y, —O—Y, —O-L-Y, —O-L-Y-L-Y, and —S(O)ₚR^{5c}.

7. A compound of any one of claims 1-3 wherein R³ is attached to the phenyl ring through a nitrogen atom and is —NO₂, —NR^{5a}R^{5b}, —NHC(O)NR^{5a}R^{5b}, —NHC(O)R^{5c}, or —NHC(O)Y.

8. A compound of claim 7 wherein R³ is selected from a group consisting of

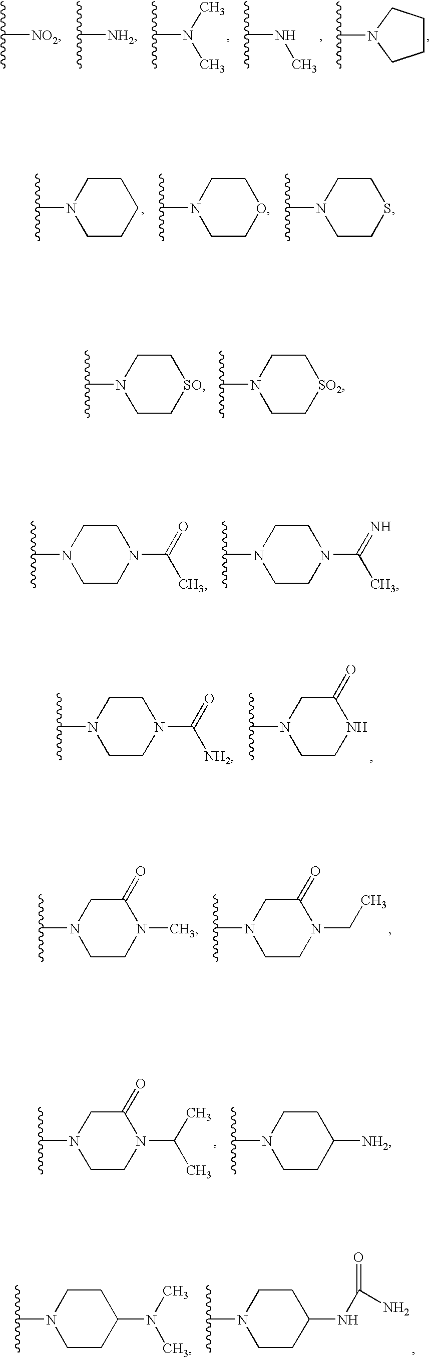

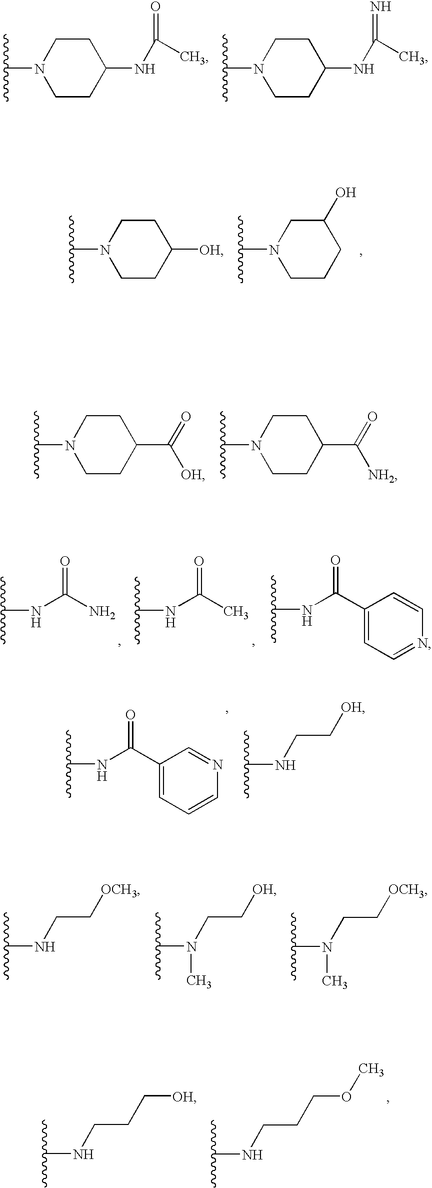

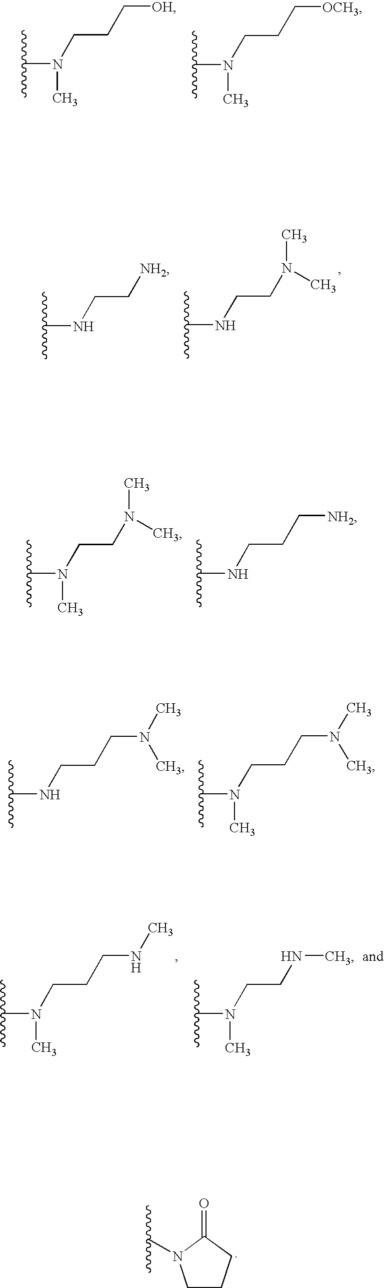

9. A compound of any one of claims 1-3 wherein R³ is optionally substituted aryl or heteroaryl.

10. A compound of claim 9 wherein R³ is selected from a group consisting of:

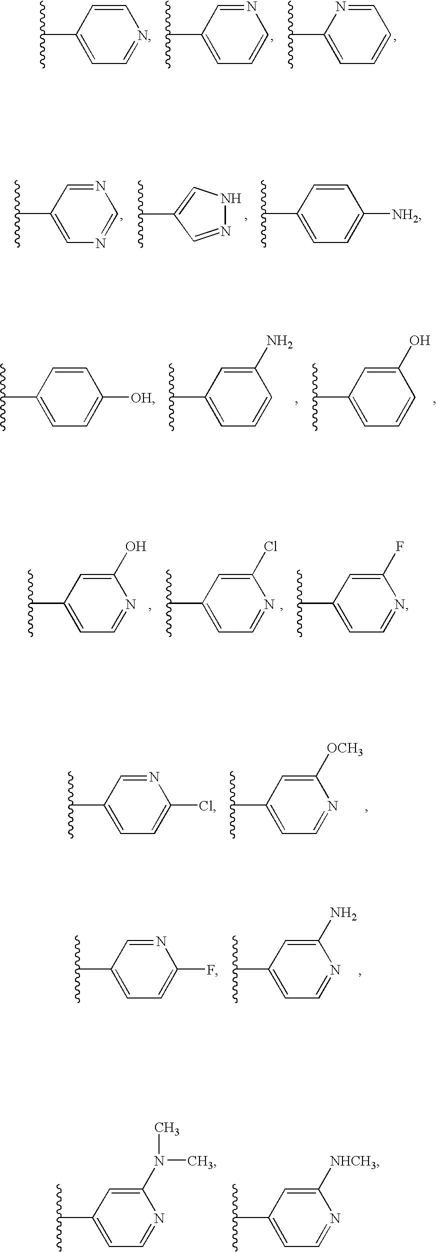

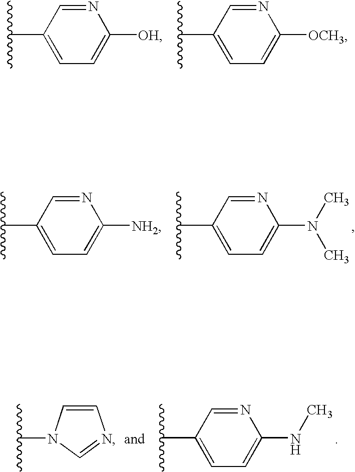

11. A compound of any one of claims 1-3 wherein R³ is attached to the phenyl ring through a carbon atom and is selected from the group consisting of optionally substituted C₁₋₆ alkyl, -L-NR^{5a}R^{5b}, —CO₂H, —C(O)NR^{5a}R^{5b}, —C(O)NR^{5a}Y, and —C(O)NH-L-Y.

12. A compound of claim 11 wherein R³ is selected from a group consisting of:

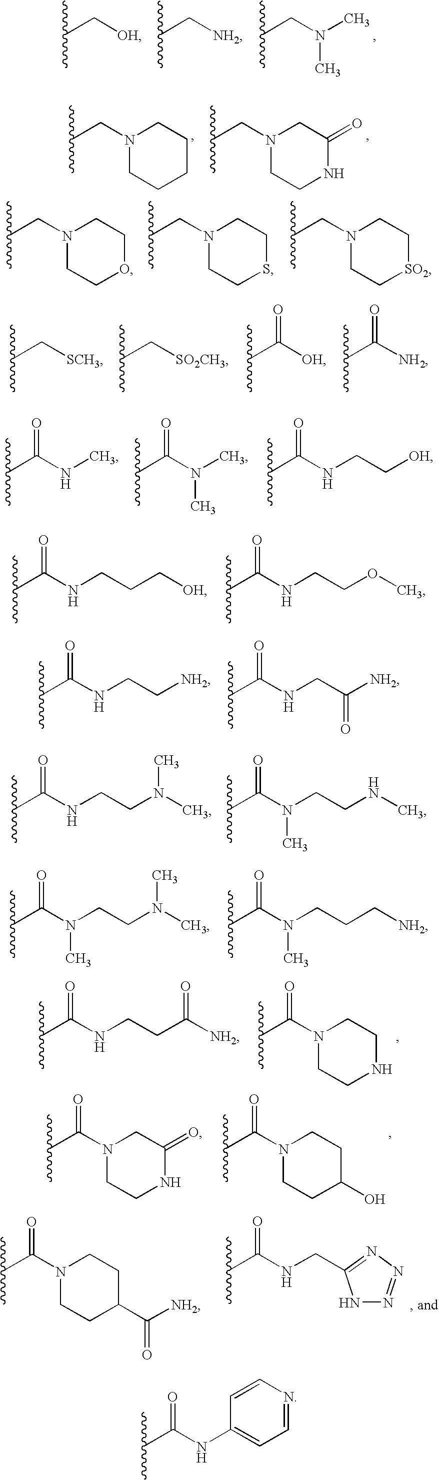

13. A compound of any one of claims 1-3 wherein R³ is attached to the phenyl ring through an oxygen atom and is selected from the group consisting of optionally substituted C₁₋₆ alkoxy, OH, —O-L-NR^{5a}R^{5b}, —O-L-O—C(O)NR^{5a}R^{5b}, —O—Y, —O-L-Y and —O-L-Y-L-Y.

14. A compound of claim 13 wherein R³ is selected from a group consisting of:

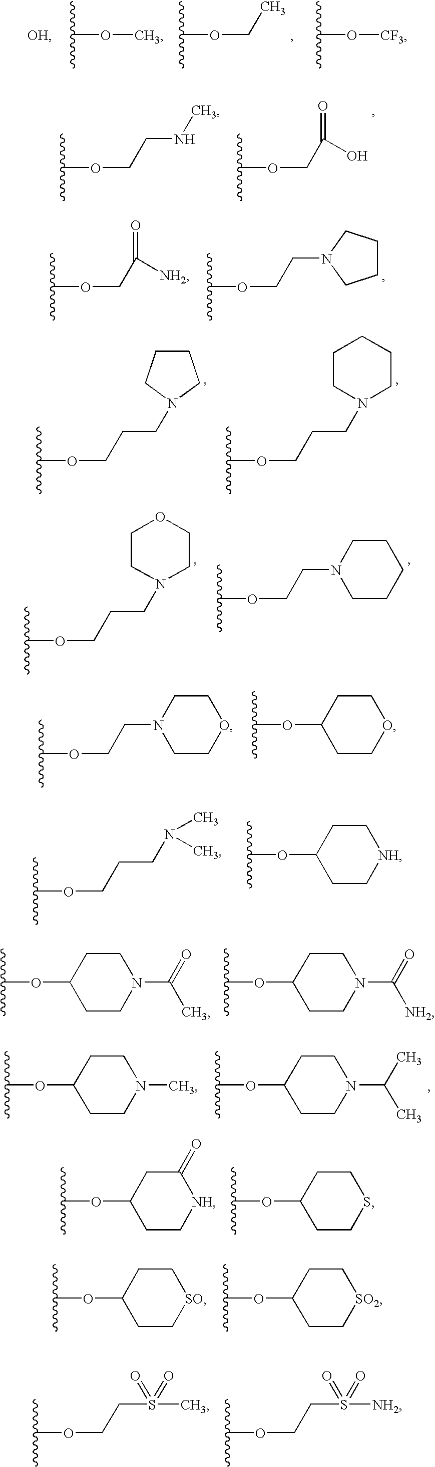

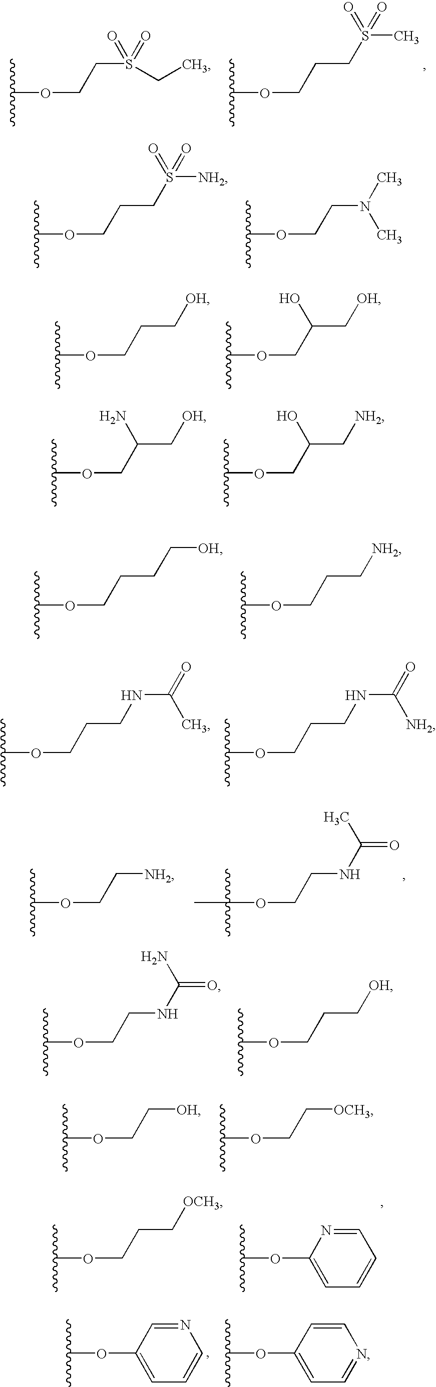

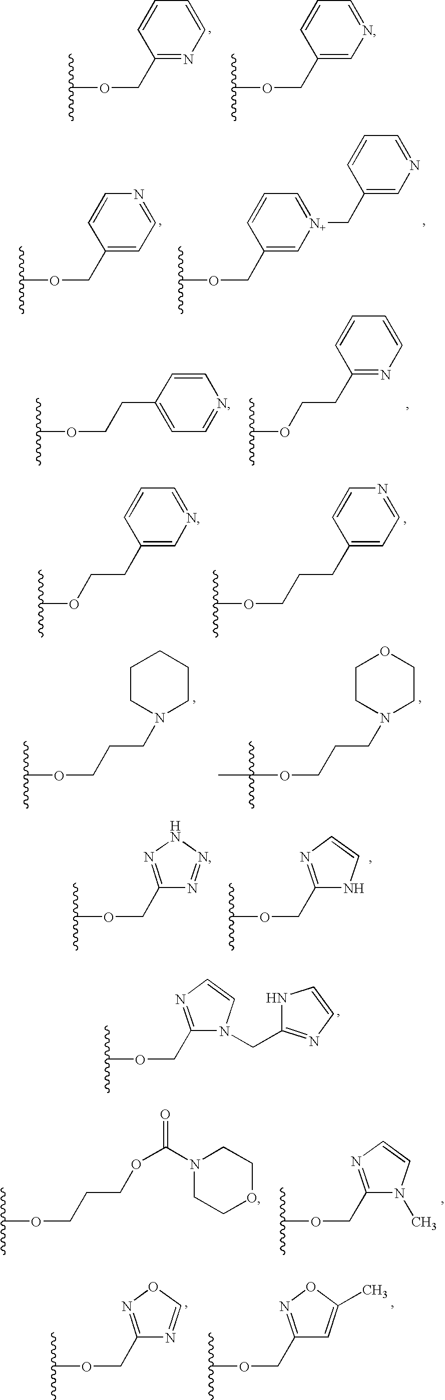

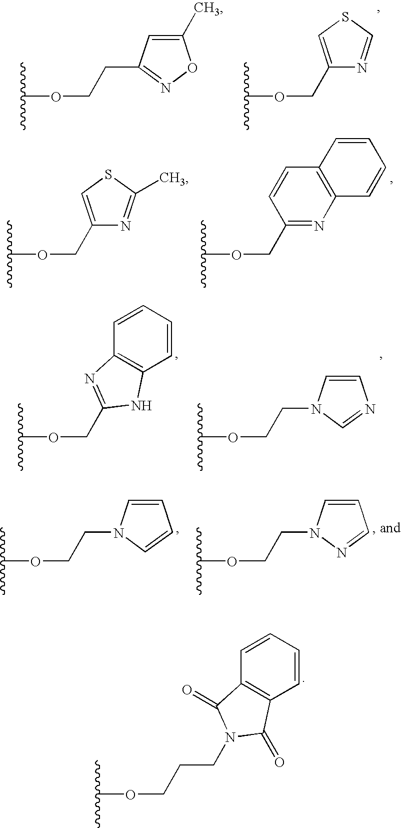

15. A compound of any one of claims 1-3 wherein R³ is attached to the phenyl ring through a sulfur atom and is —S(O)ₚR^{5c}.

16. A compound of claim 15 wherein R³ is selected from a group consisting of:

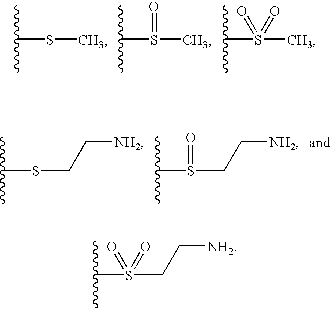

17. A compound that is selected from the group consisting of:
5-Chloro-N-((1-(2-(methylamino)-4-(2-oxopyridin-1(2H)-yl)phenyl)-1H-1,2,3-triazol-4-yl)methyl)thiophene-2-carboxamide,
5-Chloro-N-((1-(2-(dimethylamino)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(pyrrolidin-1-yl)phenyl)-1H-1,2,3-triazol-4-yl)methyl)thiophene-2-carboxamide,
5-Chloro-N-((1-(4-(2-oxopyridin-1(2H)-yl)-2-(piperidin-1-yl)phenyl)-1H-1,2,3-triazol-4-yl)methyl)thiophene-2-carboxamide,
5-Chloro-N-((1-(2-morpholino-4-(2-oxopyridin-1(2H)-yl)phenyl)-1H-1,2,3-triazol-4-yl)methyl)thiophene-2-carboxamide,
5-Chloro-N-((1-(2-(3-oxopiperazin-1-yl)-4-(2-oxopyridin-1(2H)-yl)phenyl)-1H-1,2,3-triazol-4-yl)methyl)thiophene-2-carboxamide,
5-Chloro-N-((1-(2-(4-methyl-3-oxopiperazin-1-yl)-4-(2-oxopyridin-1(2H)-yl)phenyl)-1H-1,2,3-triazol-4-yl)methyl)thiophene-2-carboxamide,
5-Chloro-N-((1-(2-(4-ethyl-3-oxopiperazin-1-yl)-4-(2-oxopyridin-1(2H)-yl)phenyl)-1H-1,2,3-triazol-4-yl)methyl)thiophene-2-carboxamide,
5-Chloro-N-((1-(2-(4-isopropyl-3-oxopiperazin-1-yl)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-thiomorpholinophenyl)-1H-1,2,3-triazol-4-yl)methyl)thiophene-2-carboxamide,
5-Chloro-N-((1-(4-(2-oxopyridin-1(2H)-yl)-2-((1-oxo-)thiomorpholino)phenyl)-1H-1,2,3-triazol-4-yl)methyl)thiophene-2-carboxamide,
5-Chloro-N-((1-(4-(2-oxopyridin-1(2H)-yl)-2-((1,1-dioxo-)thiomorpholino)phenyl)-1H-1,2,3-triazol-4-yl)methyl)thiophene-2-carboxamide,
N-((1-(2-(4-Acetylpiperazin-1-yl)-4-(2-oxopyridin-1(2H)-yl)phenyl)-1H-1,2,3-triazol-4-yl)methyl)-5-chlorothiophene-2-carboxamide,
5-Chloro-N-((1-(2-(4-(1-iminoethyl)piperazin-1-yl)-4-(2-oxopyridin-1(2H)-yl)phenyl)-1H-1,2,3-triazol-4-yl)methyl)thiophene-2-carboxamide,
4-(2-(4-((2-Chlorothiophene-5-carboxamido)methyl)-1H-1,2,3-triazol-1-yl)-5-(2-oxopyridin-1(2H)-yl)phenyl)piperazine-1-carboxamide,
5-Chloro-N-((1-(2-(4-(dimethylamino)piperidin-1-yl)-4-(2-oxopyridin-1(2H)-yl)phenyl)-1H-1,2,3-triazol-4-yl)methyl)thiophene-2-carboxamide,
N-((1-(2-(4-Aminopiperidin-1-yl)-4-(2-oxopyridin-1(2H)-yl)phenyl)-1H-1,2,3-triazol-4-yl)methyl)-5-chlorothiophene-2-carboxamide,
N-((1-(2-(4-Acetamidopiperidin-1-yl)-4-(2-oxopyridin-1(2H)-yl)phenyl)-1H-1,2,3-triazol-4-yl)methyl)-5-chlorothiophene-2-carboxamide,
N-((1-(2-(4-Acetamidinopiperidin-1-yl)-4-(2-oxopyridin-1(2H)-yl)phenyl)-1H-1,2,3-triazol-4-yl)methyl)-5-chlorothiophene-2-carboxamide,
1-(1-(2-(4-((2-Chlorothiophene-5-carboxamido)methyl)-1H-1,2,3-triazol-1-yl)-5-(2-oxopyridin-1(2H)-yl)phenyl)piperidin-4-yl)urea,
5-Chloro-N-((1-(2-(4-hydroxypiperidin-1-yl)-4-(2-oxopyridin-1(2H)-yl)phenyl)-1H-1,2,3-triazol-4-yl)methyl)thiophene-2-carboxamide,
5-Chloro-N-((1-(2-((R)-3-hydroxypiperidin-1-yl)-4-(2-oxopyridin-1(2H)-yl)phenyl)-1H-1,2,3-triazol-4-yl)methyl)thiophene-2-carboxamide,
5-Chloro-N-((1-(2-((S)-3-hydroxypiperidin-1-yl)-4-(2-oxopyridin-1(2H)-yl)phenyl)-1H-1,2,3-triazol-4-yl)methyl)thiophene-2-carboxamide,
1-(2-(4-((2-Chlorothiophene-5-carboxamido)methyl)-1H-1,2,3-triazol-1-yl)-5-(2-oxopyridin-1(2H)-yl)phenyl)piperidine-4-carboxylic acid,
1-(2-(4-((2-Chlorothiophene-5-carboxamido)methyl)-1H-1,2,3-triazol-1-yl)-5-(2-oxopyridin-1(2H)-yl)phenyl)piperidine-4-carboxamide,
5-Chloro-N-((1-(2-(2-hydroxyethylamino)-4-(2-oxopyridin-1(2H)-yl)phenyl)-1H-1,2,3-triazol-4-yl)methyl)thiophene-2-carboxamide,
5-Chloro-N-((1-(2-(2-methoxyethylamino)-4-(2-oxopyridin-1(2H)-yl)phenyl)-1H-1,2,3-triazol-4-yl)methyl)thiophene-2-carboxamide,
5-Chloro-N-((1-(2-((2-hydroxyethyl)(methyl)amino)-4-(2-oxopyridin-1(2H)-yl)phenyl)-1H-1,2,3-triazol-4-yl)methyl)thiophene-2-carboxamide,
5-Chloro-N-((1-(2-((2-methoxyethyl)(methyl)amino)-4-(2-oxopyridin-1(2H)-yl)phenyl)-1H-1,2,3-triazol-4-yl)methyl)thiophene-2-carboxamide,
N-((1-(2-(2-Aminoethylamino)-4-(2-oxopyridin-1(2H)-yl)phenyl)-1H-1,2,3-triazol-4-yl)methyl)-5-chlorothiophene-2-carboxamide,
5-Chloro-N-((1-(2-(2-(dimethylamino)ethylamino)-4-(2-oxopyridin-1(2H)-yl)phenyl)-1H-1,2,3-triazol-4-yl)methyl)thiophene-2-carboxamide,
5-Chloro-N-((1-(2-((2-(dimethylamino)ethyl)(methyl)amino)-4-(2-oxopyridin-1(2H)-yl)phenyl)-1H-1,2,3-triazol-4-yl)methyl)thiophene-2-carboxamide,
5-Chloro-N-((1-(2-(3-hydroxypropylamino)-4-(2-oxopyridin-1(2H)-yl)phenyl)-1H-1,2,3-triazol-4-yl)methyl)thiophene-2-carboxamide,
5-Chloro-N-((1-(2-(3-methoxypropylamino)-4-(2-oxopyridin-1(2H)-yl)phenyl)-1H-1,2,3-triazol-4-yl)methyl)thiophene-2-carboxamide,
5-Chloro-N-((1-(2-((3-hydroxypropyl)(methyl)amino)-4-(2-oxopyridin-1(2H)-yl)phenyl)-1H-1,2,3-triazol-4-yl)methyl)thiophene-2-carboxamide,
5-Chloro-N-((1-(2-((3-methoxypropyl)(methyl)amino)-4-(2-oxopyridin-1(2H)-yl)phenyl)-1H-1,2,3-triazol-4-yl)methyl)thiophene-2-carboxamide,
N-((1-(2-(3-Aminopropylamino)-4-(2-oxopyridin-1(2H)-yl)phenyl)-1H-1,2,3-triazol-4-yl)methyl)-5-chlorothiophene-2-carboxamide,
5-Chloro-N-((1-(2-(3-(dimethylamino)propylamino)-4-(2-oxopyridin-1(2H)-yl)phenyl)-1H-1,2,3-triazol-4-yl)methyl)thiophene-2-carboxamide,
5-Chloro-N-((1-(2-((3-(dimethylamino)propyl)(methyl)amino)-4-(2-oxopyridin-1(2H)-yl)phenyl)-1H-1,2,3-triazol-4-yl)methyl)thiophene-2-carboxamide,
5-Chloro-N-((1-(2-(methyl(3-(methylamino)propyl)amino)-4-(2-oxopyridin-1(2H)-yl)phenyl)-1H-1,2,3-triazol-4-yl)methyl)thiophene-2-carboxamide,
5-Chloro-N-((1-(2-(methyl(2-(methylamino)ethyl)amino)-4-(2-oxopyridin-1(2H)-yl)phenyl)-1H-1,2,3-triazol-4-yl)methyl)thiophene-2-carboxamide,
N-((1-(2-(1H-Imidazol-1-yl)-4-(2-oxopyridin-1(2H)-yl)phenyl)-1H-1,2,3-triazol-4-yl)methyl)-5-chlorothiophene-2-carboxamide,
5-Chloro-N-((1-(4-(2-oxopyridin-1(2H)-yl)-2-(2-oxopyrrolidin-1-yl)phenyl)-1H-1,2,3-triazol-4-yl)methyl)thiophene-2-carboxamide,
5-Chloro-N-((1-(2-nitro-4-(2-oxopyridin-1(2H)-yl)phenyl)-1H-1,2,3-triazol-4-yl)methyl)thiophene-2-carboxamide,
N-((1-(2-Amino-4-(2-oxopyridin-1(2H)-yl)phenyl)-1H-1,2,3-triazol-4-yl)methyl)-5-chlorothiophene-2-carboxamide,
1-(2-(4-((2-Chlorothiophene-5-carboxamido)methyl)-1H-1,2,3-triazol-1-yl)-5-(2-oxopyridin-1(2H)-yl)phenyl)urea,
N-((1-(2-Acetamido-4-(2-oxopyridin-1(2H)-yl)phenyl)-1H-1,2,3-triazol-4-yl)methyl)-5-chlorothiophene-2-carboxamide,
N-(2-(4-((2-Chlorothiophene-5-carboxamido)methyl)-1H-1,2,3-triazol-1-yl)-5-(2-oxopyridin-1(2H)-yl)phenyl)isonicotinamide,
N-(2-(4-((2-Chlorothiophene-5-carboxamido)methyl)-1H-1,2,3-triazol-1-yl)-5-(2-oxopyridin-1(2H)-yl)phenyl)nicotinamide,
5-Chloro-N-((1-(2-(methylthio)-4-(2-oxopyridin-1(2H)-yl)phenyl)-1H-1,2,3-triazol-4-yl)methyl)thiophene-2-carboxamide,
5-Chloro-N-((1-(2-(methylsulfoxy)-4-(2-oxopyridin-1(2H)-yl)phenyl)-1H-1,2,3-triazol-4-yl)methyl)thiophene-2-carboxamide,
5-Chloro-N-((1-(2-(methylsulfonyl)-4-(2-oxopyridin-1(2H)-yl)phenyl)-1H-1,2,3-triazol-4-yl)methyl)thiophene-2-carboxamide,
N-((1-(2-(2-Aminoethylthio)-4-(2-oxopyridin-1(2H)-yl)phenyl)-1H-1,2,3-triazol-4-yl)methyl)-5-chlorothiophene-2-carboxamide,
N-((1-(2-(2-Aminoethylsulfoxy)-4-(2-oxopyridin-1(2H)-yl)phenyl)-1H-1,2,3-triazol-4-yl)methyl)-5-chlorothiophene-2-carboxamide,
N-((1-(2-(2-Aminoethylsulfonyl)-4-(2-oxopyridin-1(2H)-yl)phenyl)-1H-1,2,3-triazol-4-yl)methyl)-5-chlorothiophene-2-carboxamide,
5-Chloro-N-((1-(2-methoxy-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(piperidin-4-yloxy)phenyl)-1H-1,2,3-triazol-4-yl)methyl)thiophene-2-carboxamide,
N-((1-(2-(1-Acetylpiperidin-4-yloxy)-4-(2-oxopyridin-1(2H)-yl)phenyl)-1H-1,2,3-triazol-4-yl)methyl)-5-chlorothiophene-2-carboxamide,
4-(2-(4-((2-Chlorothiophene-5-carboxamido)methyl)-1H-1,2,3-triazol-1-yl)-5-(2-oxopyridin-1(2H)-yl)phenoxy)piperidine-1-carboxamide,
5-Chloro-N-((1-(2-(1-methylpiperidin-4-yloxy)-4-(2-oxopyridin-1(2H)-yl)phenyl)-1H-1,2,3-triazol-4-yl)methyl)thiophene-2-carboxamide,
5-Chloro-N-((1-(2-(1-isopropylpiperidin-4-yloxy)-4-(2-oxopyridin-1(2H)-yl)phenyl)-1H-1,2,3-triazol-4-yl)methyl)thiophene-2-carboxamide,
5-Chloro-N-((1-(2-(2-oxopiperidin-4-yloxy)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(pyridin-4-yloxy)phenyl)-1H-1,2,3-triazol-4-yl)methyl)thiophene-2-carboxamide,
5-Chloro-N-((1-(4-(2-oxopyridin-1(2H)-yl)-2-(pyridin-3-yloxy)phenyl)-1H-1,2,3-triazol-4-yl)methyl)thiophene-2-carboxamide,
5-Chloro-N-((1-(4-(2-oxopyridin-1(2H)-yl)-2-(tetrahydro-2H-thiopyran-4-yloxy)phenyl)-1H-1,2,3-triazol-4-yl)methyl)thiophene-2-carboxamide,
5-Chloro-N-((1-(4-(2-oxopyridin-1(2H)-yl)-2-(1,1-dioxo-tetrahydro-2H-thiopyran-4-yloxy)phenyl)-1H-1,2,3-triazol-4-yl)methyl)thiophene-2-carboxamide,
5-Chloro-N-((1-(2-(3-(1,3-dioxoisoindolin-2-yl)propoxy)-4-(2-oxopyridin-1(2H)-yl)phenyl)-1H-1,2,3-triazol-4-yl)methyl)thiophene-2-carboxamide,
N-((1-(2-(3-Aminopropoxy)-4-(2-oxopyridin-1(2H)-yl)phenyl)-1H-1,2,3-triazol-4-yl)methyl)-5-chlorothiophene-2-carboxamide,
N-((1-(2-(3-Acetamidopropoxy)-4-(2-oxopyridin-1(2H)-yl)phenyl)-1H-1,2,3-triazol-4-yl)methyl)-5-chlorothiophene-2-carboxamide,
1-(3-(2-(4-((2-Chlorothiophene-5-carboxamido)methyl)-1H-1,2,3-triazol-1-yl)-5-(2-oxopyridin-1(2H)-yl)phenoxy)propyl)urea,
N-((1-(2-(2-Aminoethoxy)-4-(2-oxopyridin-1(2H)-yl)phenyl)-1H-1,2,3-triazol-4-yl)methyl)-5-chlorothiophene-2-carboxamide,
N-((1-(2-(2-Acetamidoethoxy)-4-(2-oxopyridin-1(2H)-yl)phenyl)-1H-1,2,3-triazol-4-yl)methyl)-5-chlorothiophene-2-carboxamide,
1-(2-(2-(4-((2-Chlorothiophene-5-carboxamido)methyl)-1H-1,2,3-triazol-1-yl)-5-(2-oxopyridin-1(2H)-yl)phenoxy)ethyl)urea,
5-Chloro-N-((1-(2-(3-hydroxypropoxy)-4-(2-oxopyridin-1(2H)-yl)phenyl)-1H-1,2,3-triazol-4-yl)methyl)thiophene-2-carboxamide,
5-Chloro-N-((1-(2-(2-hydroxyethoxy)-4-(2-oxopyridin-1(2H)-yl)phenyl)-1H-1,2,3-triazol-4-yl)methyl)thiophene-2-carboxamide,
5-Chloro-N-((1-(2-(2-methoxyethoxy)-4-(2-oxopyridin-1(2H)-yl)phenyl)-1H-1,2,3-triazol-4-yl)methyl)thiophene-2-carboxamide,
5-Chloro-N-((1-(2-(3-methoxypropoxy)-4-(2-oxopyridin-1(2H)-yl)phenyl)-1H-1,2,3-triazol-4-yl)methyl)thiophene-2-carboxamide,
5-Chloro-N-((1-(2-((R)-2,3-dihydroxypropoxy)-4-(2-oxopyridin-1(2H)-yl)phenyl)-1H-1,2,3-triazol-4-yl)methyl)thiophene-2-carboxamide,
5-Chloro-N-((1-(2-((S)-2,3-dihydroxypropoxy)-4-(2-oxopyridin-1(2H)-yl)phenyl)-1H-1,2,3-triazol-4-yl)methyl)thiophene-2-carboxamide,
5-Chloro-N-((1-(2-(2-(methylsulfonyl)ethoxy)-4-(2-oxopyridin-1(2H)-yl)phenyl)-1H-1,2,3-triazol-4-yl)methyl)thiophene-2-carboxamide,
5-Chloro-N-((1-(2-(2-(aminosulfonyl)ethoxy)-4-(2-oxopyridin-1(2H)-yl)phenyl)-1H-1,2,3-triazol-4-yl)methyl)thiophene-2-carboxamide,
5-Chloro-N-((1-(2-(2-(ethylsulfonyl)ethoxy)-4-(2-oxopyridin-1(2H)-yl)phenyl)-1H-1,2,3-triazol-4-yl)methyl)thiophene-2-carboxamide,
5-Chloro-N-((1-(2-(3-(methylsulfonyl)propoxy)-4-(2-oxopyridin-1(2H)-yl)phenyl)-1H-1,2,3-triazol-4-yl)methyl)thiophene-2-carboxamide,
5-Chloro-N-((1-(2-(3-(aminosulfonyl)propoxy)-4-(2-oxopyridin-1(2H)-yl)phenyl)-1H-1,2,3-triazol-4-yl)methyl)thiophene-2-carboxamide,
5-Chloro-N-((1-(2-(2-(dimethylamino)ethoxy)-4-(2-oxopyridin-1(2H)-yl)phenyl)-1H-1,2,3-triazol-4-yl)methyl)thiophene-2-carboxamide,
5-Chloro-N-((1-(2-(2-(dimethyl(dimethylamino)amino)ethoxy)-4-(2-oxopyridin-1(2H)-yl)phenyl)-1H-1,2,3-triazol-4-yl)methyl)thiophene-2-carboxamide,
5-Chloro-N-((1-(2-(2-(methylamino)ethoxy)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(2-(pyrrolidin-1-yl)ethoxy)phenyl)-1H-1,2,3-triazol-4-yl)methyl)thiophene-2-carboxamide,
5-Chloro-N-((1-(4-(2-oxopyridin-1(2H)-yl)-2-(2-(piperidin-1-yl)ethoxy)phenyl)-1H-1,2,3-triazol-4-yl)methyl)thiophene-2-carboxamide,
5-Chloro-N-((1-(2-(2-morpholinoethoxy)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(2-(pyridin-4-yl)ethoxy)phenyl)-1H-1,2,3-triazol-4-yl)methyl)thiophene-2-carboxamide,
5-Chloro-N-((1-(2-(3-(dimethylamino)propoxy)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(3-(pyrrolidin-1-yl)propoxy)phenyl)-1H-1,2,3-triazol-4-yl)methyl)thiophene-2-carboxamide,
5-Chloro-N-((1-(4-(2-oxopyridin-1(2H)-yl)-2-(3-(piperidin-1-yl)propoxy)phenyl)-1H-1,2,3-triazol-4-yl)methyl)thiophene-2-carboxamide,
5-Chloro-N-((1-(2-(3-morpholinopropoxy)-4-(2-oxopyridin-1(2H)-yl)phenyl)-1H-1,2,3-triazol-4-yl)methyl)thiophene-2-carboxamide,
2-(2-(4-((2-Chlorothiophene-5-carboxamido)methyl)-1H-1,2,3-triazol-1-yl)-5-(2-oxopyridin-1(2H)-yl)phenoxy)acetic acid,
N-((1-(2-(2-Amino-2-oxoethoxy)-4-(2-oxopyridin-1(2H)-yl)phenyl)-1H-1,2,3-triazol-4-yl)methyl)-5-chlorothiophene-2-carboxamide,
N-((1-(2-((1H-Tetrazol-5-yl)methoxy)-4-(2-oxopyridin-1(2H)-yl)phenyl)-1H-1,2,3-triazol-4-yl)methyl)-5-chlorothiophene-2-carboxamide,
5-Chloro-N-((1-(4-(2-oxopyridin-1(2H)-yl)-2-(trifluoromethoxy)phenyl)-1H-1,2,3-triazol-4-yl)methyl)thiophene-2-carboxamide,
2-(4-((2-Chlorothiophene-5-carboxamido)methyl)-1H-1,2,3-triazol-1-yl)-5-(2-oxopyridin-1(2H)-yl)benzoic acid,
N-((1-(2-Carbamoyl-4-(2-oxopyridin-1(2H)-yl)phenyl)-1H-1,2,3-triazol-4-yl)methyl)-5-chlorothiophene-2-carboxamide,
5-Chloro-N-((1-(2-(methylcarbamoyl)-4-(2-oxopyridin-1(2H)-yl)phenyl)-1H-1,2,3-triazol-4-yl)methyl)thiophene-2-carboxamide,
5-Chloro-N-((1-(2-(dimethylcarbamoyl)-4-(2-oxopyridin-1(2H)-yl)phenyl)-1H-1,2,3-triazol-4-yl)methyl)thiophene-2-carboxamide,
N-((1-(2-((2-Hydroxyethyl)carbamoyl)-4-(2-oxopyridin-1(2H)-yl)phenyl)-1H-1,2,3-triazol-4-yl)methyl)-5-chlorothiophene-2-carboxamide,
N-((1-(2-((3-Hydroxypropyl)carbamoyl)-4-(2-oxopyridin-1(2H)-yl)phenyl)-1H-1,2,3-triazol-4-yl)methyl)-5-chlorothiophene-2-carboxamide,
N-((1-(2-((2-Methoxyethyl)carbamoyl)-4-(2-oxopyridin-1(2H)-yl)phenyl)-1H-1,2,3-triazol-4-yl)methyl)-5-chlorothiophene-2-carboxamide,
N-((1-(2-((2-Aminoethyl)carbamoyl)-4-(2-oxopyridin-1(2H)-yl)phenyl)-1H-1,2,3-triazol-4-yl)methyl)-5-chlorothiophene-2-carboxamide,
N-((1-(2-((2-Amino-2-oxoethyl)carbamoyl)-4-(2-oxopyridin-1(2H)-yl)phenyl)-1H-1,2,3-triazol-4-yl)methyl)-5-chlorothiophene-2-carboxamide,
N-((1-(2-((2-(Dimethylamino)ethyl)carbamoyl)-4-(2-oxopyridin-1(2H)-yl)phenyl)-1H-1,2,3-triazol-4-yl)methyl)-5-chlorothiophene-2-carboxamide,
5-Chloro-N-((1-(2-(methyl(2-(methylamino)ethyl)carbamoyl)-4-(2-oxopyridin-1(2H)-yl)phenyl)-1H-1,2,3-triazol-4-yl)methyl)thiophene-2-carboxamide,
5-Chloro-N-((1-(2-((2-(dimethylamino)ethyl)(methyl)carbamoyl)-4-(2-oxopyridin-1(2H)-yl)phenyl)-1H-1,2,3-triazol-4-yl)methyl)thiophene-2-carboxamide,
N-((1-(2-(3-aminopropylcarbamoyl)-4-(2-oxopyridin-1(2H)-yl)phenyl)-1H-1,2,3-triazol-4-yl)methyl)-5-chlorothiophene-2-carboxamide,
5-Chloro-N-((1-(4-(2-oxopyridin-1(2H)-yl)-2-(piperazine-1-carbonyl)phenyl)-1H-1,2,3-triazol-4-yl)methyl)thiophene-2-carboxamide,
5-Chloro-N-((1-(2-(2-oxopiperazine-4-carbonyl)-4-(2-oxopyridin-1(2H)-yl)phenyl)-1H-1,2,3-triazol-4-yl)methyl)thiophene-2-carboxamide,
5-Chloro-N-((1-(2-(4-hydroxypiperidine-1-carbonyl)-4-(2-oxopyridin-1(2H)-yl)phenyl)-1H-1,2,3-triazol-4-yl)methyl)thiophene-2-carboxamide,
1-(2-(4-((5-Chlorothiophene-2-carboxamido)methyl)-1H-1,2,3-triazol-1-yl)-5-(2-oxopyridin-1(2H)-yl)benzoyl)piperidine-4-carboxamide,
N-((1-(2-(((1H-Tetrazol-5-yl)methyl)carbamoyl)-4-(2-oxopyridin-1(2H)-yl)phenyl)-1H-1,2,3-triazol-4-yl)methyl)-5-chlorothiophene-2-carboxamide,
5-Chloro-N-((1-(4-(2-oxopyridin-1(2H)-yl)-2-(pyridin-4-ylcarbamoyl)phenyl)-1H-1,2,3-triazol-4-yl)methyl)thiophene-2-carboxamide,
5-Chloro-N-((1-(2-(hydroxymethyl)-4-(2-oxopyridin-1(2H)-yl)phenyl)-1H-1,2,3-triazol-4-yl)methyl)thiophene-2-carboxamide,
N-((1-(2-(Aminomethyl)-4-(2-oxopyridin-1(2H)-yl)phenyl)-1H-1,2,3-triazol-4-yl)methyl)-5-chlorothiophene-2-carboxamide,
5-Chloro-N-((1-(2-((dimethylamino)methyl)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(piperidin-1-ylmethyl)phenyl)-1H-1,2,3-triazol-4-yl)methyl)thiophene-2-carboxamide,
5-Chloro-N-((1-(2-(methylthiomethyl)-4-(2-oxopyridin-1(2H)-yl)phenyl)-1H-1,2,3-triazol-4-yl)methyl)thiophene-2-carboxamide,
5-Chloro-N-((1-(2-(methylsulfonylmethyl)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(pyridin-4-yl)phenyl)-1H-1,2,3-triazol-4-yl)methyl)thiophene-2-carboxamide,
5-Chloro-N-((1-(4-(2-oxopyridin-1(2H)-yl)-2-(pyridin-3-yl)phenyl)-1H-1,2,3-triazol-4-yl)methyl)thiophene-2-carboxamide,
5-Chloro-N-((1-(4-(2-oxopyridin-1(2H)-yl)-2-(pyrimidin-5-yl)phenyl)-1H-1,2,3-triazol-4-yl)methyl)thiophene-2-carboxamide,
5-Chloro-N-((1-(4-(2-oxopyridin-1(2H)-yl)-2-(1H-pyrazol-3-yl)phenyl)-1H-1,2,3-triazol-4-yl)methyl)thiophene-2-carboxamide,
5-Chloro-N-((1-(4-(2-oxopyridin-1(2H)-yl)-2-((4-aminophenyl)phenyl)-1H-1,2,3-triazol-4-yl)methyl)thiophene-2-carboxamide,
5-Chloro-N-((1-(4-(2-oxopyridin-1(2H)-yl)-2-((4-hydroxyphenyl)phenyl)-1H-1,2,3-triazol-4-yl)methyl)thiophene-2-carboxamide,
5-Chloro-N-((1-(4-(2-oxopyridin-1(2H)-yl)-2-((3-aminophenyl)phenyl)-1H-1,2,3-triazol-4-yl)methyl)thiophene-2-carboxamide,
5-Chloro-N-((1-(4-(2-oxopyridin-1(2H)-yl)-2-((3-hydroxyphenyl)phenyl)-1H-1,2,3-triazol-4-yl)methyl)thiophene-2-carboxamide,
5-Chloro-N-((1-(2-(2-chloropyridin-4-yl)-4-(2-oxopyridin-1(2H)-yl)phenyl)-1H-1,2,3-triazol-4-yl)methyl)thiophene-2-carboxamide,
5-Chloro-N-((1-(2-(2-fluoropyridin-4-yl)-4-(2-oxopyridin-1(2H)-yl)phenyl)-1H-1,2,3-triazol-4-yl)methyl)thiophene-2-carboxamide,
5-Chloro-N-((1-(2-(6-chloropyridin-3-yl)-4-(2-oxopyridin-1(2H)-yl)phenyl)-1H-1,2,3-triazol-4-yl)methyl)thiophene-2-carboxamide,
5-Chloro-N-((1-(2-(6-fluoropyridin-3-yl)-4-(2-oxopyridin-1(2H)-yl)phenyl)-1H-1,2,3-triazol-4-yl)methyl)thiophene-2-carboxamide,
5-Chloro-N-((1-(2-(2-hydroxypyridin-4-yl)-4-(2-oxopyridin-1(2H)-yl)phenyl)-1H-1,2,3-triazol-4-yl)methyl)thiophene-2-carboxamide,
5-Chloro-N-((1-(2-(2-methoxypyridin-4-yl)-4-(2-oxopyridin-1(2H)-yl)phenyl)-1H-1,2,3-triazol-4-yl)methyl)thiophene-2-carboxamide,
N-((1-(2-(2-Aminopyridin-4-yl)-4-(2-oxopyridin-1(2H)-yl)phenyl)-1H-1,2,3-triazol-4-yl)methyl)-5-chlorothiophene-2-carboxamide,
5-Chloro-N-((1-(2-(2-(dimethylamino)pyridin-4-yl)-4-(2-oxopyridin-1(2H)-yl)phenyl)-1H-1,2,3-triazol-4-yl)methyl)thiophene-2-carboxamide,
5-Chloro-N-((1-(2-(2-(methylamino)pyridin-4-yl)-4-(2-oxopyridin-1(2H)-yl)phenyl)-1H-1,2,3-triazol-4-yl)methyl)thiophene-2-carboxamide,
5-Chloro-N-((1-(2-(6-hydroxypyridin-3-yl)-4-(2-oxopyridin-1(2H)-yl)phenyl)-1H-1,2,3-triazol-4-yl)methyl)thiophene-2-carboxamide,
5-Chloro-N-((1-(2-(6-methoxypyridin-3-yl)-4-(2-oxopyridin-1(2H)-yl)phenyl)-1H-1,2,3-triazol-4-yl)methyl)thiophene-2-carboxamide,
N-((1-(2-(6-Aminopyridin-3-yl)-4-(2-oxopyridin-1(2H)-yl)phenyl)-1H-1,2,3-triazol-4-yl)methyl)-5-chlorothiophene-2-carboxamide,
5-Chloro-N-((1-(2-(6-(dimethylamino)pyridin-3-yl)-4-(2-oxopyridin-1(2H)-yl)phenyl)-1H-1,2,3-triazol-4-yl)methyl)thiophene-2-carboxamide,
5-Chloro-N-((1-(2-(6-(methylamino)pyridin-3-yl)-4-(2-oxopyridin-1(2H)-yl)phenyl)-1H-1,2,3-triazol-4-yl)methyl)thiophene-2-carboxamide,
N-((1-(2-(3-Amino-3-oxopropylcarbamoyl)-4-(2-oxopyridin-1(2H)-yl)phenyl)-1H-1,2,3-triazol-4-yl)methyl)-5-chlorothiophene-2-carboxamide,
N-((1-(2-(2-(1H-Imidazol-1-yl)ethoxy)-4-(2-oxopyridin-1(2H)-yl)phenyl)-1H-1,2,3-triazol-4-yl)methyl)-5-chlorothiophene-2-carboxamide,
5-Chloro-N-((1-(4-(2-oxopyridin-1(2H)-yl)-2-(2-(pyridin-4-yl)ethoxy)phenyl)-1H-1,2,3-triazol-4-yl)methyl)thiophene-2-carboxamide,
3-(2-(4-((5-Chlorothiophene-2-carboxamido)methyl)-1H-1,2,3-triazol-1-yl)-5-(2-oxopyridin-1(2H)-yl)phenoxy)propyl morpholine-4-carboxylate,
5-Chloro-N-((1-(4-(2-oxopyridin-1(2H)-yl)-2-(pyridin-2-yloxy)phenyl)-1H-1,2,3-triazol-4-yl)methyl)thiophene-2-carboxamid,
2-(2-(4-((5-Chlorothiophene-2-carboxamido)methyl)-5-iodo-1H-1,2,3-triazol-1-yl)-5-(2-oxopyridin-1(2H)-yl)phenoxy)acetic acid,
5-Chloro-N-((1-(2-(3-hydroxypropoxy)-4-(2-oxopyridin-1(2H)-yl)phenyl)-5-iodo-1H-1,2,3-triazol-4-yl)methyl)thiophene-2-carboxamide,
5-Chloro-N-((1-(2-((R)-2,3-dihydroxypropoxy)-4-(2-oxopyridin-1(2H)-yl)phenyl)-5-iodo-1H-1,2,3-triazol-4-yl)methyl)thiophene-2-carboxamide,
5-Chloro-N-((1-(2-((S)-2,3-dihydroxypropoxy)-4-(2-oxopyridin-1(2H)-yl)phenyl)-5-iodo-1H-1,2,3-triazol-4-yl)methyl)thiophene-2-carboxamide,
N-((1-(2-(2-(1H-Pyrazol-1-yl)ethoxy)-4-(2-oxopyridin-1(2H)-yl)phenyl)-1H-1,2,3-triazol-4-yl)methyl)-5-chlorothiophene-2-carboxamide,
5-Chloro-N-((1-(4-(2-oxopiperidin-1-yl)-2-thiomorpholinophenyl)-1H-1,2,3-triazol-4-yl)methyl)thiophene-2-carboxamide,
5-Chloro-N-((1-(4-(2-oxopiperidin-1-yl)-2-((1,1-dioxo)thiomorpholino)phenyl)-1H-1,2,3-triazol-4-yl)methyl)thiophene-2-carboxamide,
5-Chloro-N-((1-(2-(3-oxopiperazin-1-yl)-4-(2-oxopiperidin-1-yl)phenyl)-1H-1,2,3-triazol-4-yl)methyl)thiophene-2-carboxamide,
5-Chloro-N-((1-(2-(morpholinomethyl)-4-(2-oxopyridin-1(2H)-yl)phenyl)-1H-1,2,3-triazol-4-yl)methyl)thiophene-2-carboxamide,
5-Chloro-N-((1-(2-((3-oxopiperazin-1-yl)methyl)-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(thiomorpholinomethyl)phenyl)-1H-1,2,3-triazol-4-yl)methyl)thiophene-2-carboxamide,
5-Chloro-N-((1-(4-(2-oxopyridin-1(2H)-yl)-2-(1,1,-dioxothiomorpholinomethyl)phenyl)-1H-1,2,3-triazol-4-yl)methyl)thiophene-2-carboxamide,
5-Chloro-N-((1-(2-ethoxy-4-(2-oxopyridin-1(2H)-yl)phenyl)-1H-1,2,3-triazol-4-yl)methyl)thiophene-2-carboxamide,
5-Chloro-N-((1-(4-(2-oxopyridin-1(2H)-yl)-2-(pyridin-3-ylmethoxy)phenyl)-1H-1,2,3-triazol-4-yl)methyl)thiophene-2-carboxamide,
5-Chloro-N-((1-(4-(2-oxopyridin-1(2H)-yl)-2-(N-(pyridine-3-yl)pyridin-3-ylmethoxy)phenyl)-1H-1,2,3-triazol-4-yl)methyl)thiophene-2-carboxamide,
5-Chloro-N-((1-(4-(2-oxopyridin-1(2H)-yl)-2-(pyridin-4-ylmethoxy)phenyl)-1H-1,2,3-triazol-4-yl)methyl)thiophene-2-carboxamide,
5-Chloro-N-((1-(4-(2-oxopyridin-1(2H)-yl)-2-(2-(pyridin-2-yl)ethoxy)phenyl)-1H-1,2,3-triazol-4-yl)methyl)thiophene-2-carboxamide,
5-Chloro-N-((1-(4-(2-oxopyridin-1(2H)-yl)-2-(pyridin-2-ylmethoxy)phenyl)-1H-1,2,3-triazol-4-yl)methyl)thiophene-2-carboxamide,
5-Chloro-N-((1-(4-(2-oxopyridin-1(2H)-yl)-2-(quinolin-2-ylmethoxy)phenyl)-1H-1,2,3-triazol-4-yl)methyl)thiophene-2-carboxamide,
5-Chloro-N-((1-(4-(2-oxopyridin-1(2H)-yl)-2-(thiazol-4-ylmethoxy)phenyl)-1H-1,2,3-triazol-4-yl)methyl)thiophene-2-carboxamide,
5-Chloro-N-((1-(2-((2-methylthiazol-4-yl)methoxy)-4-(2-oxopyridin-1(2H)-yl)phenyl)-1H-1,2,3-triazol-4-yl)methyl)thiophene-2-carboxamide,
N-((1-(2-((1H-Benzo[d]imidazol-2-yl)methoxy)-4-(2-oxopyridin-1(2H)-yl)phenyl)-1H-1,2,3-triazol-4-yl)methyl)-5-chlorothiophene-2-carboxamide,
5-Chloro-N-((1-(4-(2-oxopyridin-1(2H)-yl)-2-(2-(pyridin-3-yl)ethoxy)phenyl)-1H-1,2,3-triazol-4-yl)methyl)thiophene-2-carboxamide,
N-((1-(2-((1,2,4-Oxadiazol-3-yl)methoxy)-4-(2-oxopyridin-1(2H)-yl)phenyl)-1H-1,2,3-triazol-4-yl)methyl)-5-chlorothiophene-2-carboxamide,
5-Chloro-N-((1-(2-((1-methyl-1H-imidazol-2-yl)methoxy)-4-(2-oxopyridin-1(2H)-yl)phenyl)-1H-1,2,3-triazol-4-yl)methyl)thiophene-2-carboxamide,
N-((1-(2-((1H-Imidazol-2-yl)methoxy)-4-(2-oxopyridin-1(2H)-yl)phenyl)-1H-1,2,3-triazol-4-yl)methyl)-5-chlorothiophene-2-carboxamide,
N-((1-(2-((1-((1H-imidazol-2-yl)methyl)-1H-imidazol-2-yl)methoxy)-4-(2-oxopyridin-1(2H)-yl)phenyl)-1H-1,2,3-triazol-4-yl)methyl)-5-chlorothiophene-2-carboxamide,
5-Chloro-N-((1-(2-((5-methylisoxazol-3-yl)methoxy)-4-(2-oxopyridin-1(2H)-yl)phenyl)-1H-1,2,3-triazol-4-yl)methyl)thiophene-2-carboxamide,
N-((1-(2-(2-(1H-Pyrrol-1-yl)ethoxy)-4-(2-oxopyridin-1(2H)-yl)phenyl)-1H-1,2,3-triazol-4-yl)methyl)-5-chlorothiophene-2-carboxamide,
or a pharmaceutically acceptable salt, ester, or prodrug thereof.

18. A composition comprising a pharmaceutically acceptable excipient and a compound of claim 1 or 17.

19. A method for preventing or treating a condition in a mammal characterized by undesired thrombosis comprising the step of administering to said mammal a therapeutically effective amount of a compound of claim 1 or 17.

20. The method in accordance with claim 19, wherein the condition is selected from the group consisting of acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

21. A method for inhibiting the coagulation of a blood sample comprising contacting said sample with a compound of claim 1 or 17.
